SEQUENCE LISTING (110 sequences)

SEQ ID NO: 1 - DNA, 136 bp, Artificial
  Consensus sugar cane satellite repeat sequence
cgtcacctaggagtaccatcgggtgcgtccaaaatgatttctgagcctatggtacgtttggcgcaaaccgtgcacctatcttgcgccaacactaacactatctccaaacggaccgaaacgagattccacatgagcc

SEQ ID NO: 2 - DNA, 135 bp, Artificial
  Consensus sugar cane satellite repeat sequence
cgtcacctaggagtaccatcgggtgcgtccaaaatgatttctgagcctatggtacgttcggcgcaaaccatgcacctatcttgcatcaagattagcactatctccaaacggactgaaacgagcttccacttgacc

SEQ ID NO: 3 - DNA, 137 bp, Artificial
  Consensus sugar cane satellite repeat sequence
cgtcacctaggagttccatcgggtgcgtccaaaatgatttctgagcctatggtacgtttgacgcaaaccgtgcacctatcttgcaccaacactaacactatctccaaacggaaagaagcgagattccacatgaccca

SEQ ID NO: 4 - DNA, 137 bp, Artificial
  Consensus sugar cane satellite repeat sequence
cgtcacctaggagtaccatcgggtgcgtccaaaacgatttctgagcctatggtacgtttggcgcaaaccgtgcacctatcttgcaccaacactaacactatctccaaatggaccgaaacgagattccagatgaccca

SEQ ID NO: 5 - DNA, 137 bp, Artificial
  Consensus sugar cane satellite repeat sequence
tcacctaggagtaccatcgggtgcgtccaaaacgatttctgagcctatggtacgttcggcgcaaaccgtgcacctatcttgcaccaacactaacactatctccaaacggaccgaaacgagattccagatgacccacg

SEQ ID NO: 6 - DNA, 135 bp, Artificial
  Consensus sugar cane satellite repeat sequence
ccgtcacctaggagttccatcgggtgcgtccaaaatgatttctgagcctatggtacgtttggcgcaaaccgtgcacctatcttgcgtcaagattagcactatctccaaacggactgaatcgagcttccacttgag

SEQ ID NO: 7 - DNA, 136 bp, Artificial
  Consensus sugar cane satellite repeat sequence
tcacctaggagttccatcgggtgcgtccaaaactatttatgagcctatggtacgttcgatgcaaaccgtgcacctatcttgcatcaagactagcactatctccaaacggaccgaaccgagattccacatgaccccg

SEQ ID NO: 8 - DNA, 135 bp, Artificial
  Consensus sugar cane satellite repeat sequence
cgtcacctaggagtwccatcgggtgcgtccaaattgatttcygagcctatggtacgttcggcgcaaaccgtgcaactatcttgcaycracactaacactatctccaaacagaccgaaacaagattccacwtgasm

SEQ ID NO: 9 - DNA, 133 bp, Artificial
  Consensus sugar cane satellite repeat sequence
cgtcacctaggagtaccatcgggtgtgcccaaaatgatttccaagcctatggtacattcggcgcaaaccgtgcacctatcttgcgtcgagattaacactatctccaaacggaccgaaacgagcttccacttga

SEQ ID NO: 10 - DNA, 136 bp, Artificial
  Consensus sugar cane satellite repeat sequence
gtcacctaggagttccatcgggtgcgcccaaaatgatttccgagcctatggtacgttcggcgcaaaccgtgcacctatcttgcgtcgagattaacactatctccaaacagaccgaaacgagcttccacttgaccca

SEQ ID NO: 11 - DNA, 136 bp, Artificial
  Consensus sugar cane satellite repeat sequence
cgtcacctaggagttccatcgggtgcgtccaaaatgatttctgagcctatggtacgtttggcgcaaaccgtgcacctatcttgcaccaagactaacactatctccaaacggaccgaaacgagattccacatgaccc

SEQ ID NO: 12 - DNA, 133 bp, Artificial
  Consensus sugar cane satellite repeat sequence
tgcgtccaaaacgatttctgagcctatggtacgtttggtgcaaaccgtgcacctatcttgtgtcaagagtagcactatctccaaacggaccgaaagagcttccacaagcccacgtcacctagggttccatcgg

SEQ ID NO: 13 - DNA, 136 bp, Artificial
  Mini-chromosome monomer sequence
cgtcacctaggagttgcatcgggtgcgtccaaaatgatttttgagcctatggtacgttcggcgcaaaccgtgcaaccatcttgcatcaagattgacactatctccaaatggaccaaaacgagcttccacttgagcc

SEQ ID NO: 14 - DNA, 131 bp, Artificial
  Mini-chromosome monomer sequence
acctatgtgttccatcgggtgtgtccaaccgatttctaagcctatggtacgtttagcgcaaaccatgcacctatcttgcgtcaagactggcactatctccaaacggaccaaaacgagcttccacttaagcc

SEQ ID NO: 15 - DNA, 134 bp, Artificial
  Mini-chromosome monomer sequence
tcacctaggagtaccatcgggtgcgtccaaaatgattactaagcctatggtacgtttggcacaaaccgtgcacctatcttgagtcaagattaacactatctgcaaacggaccgaaatgagcttccacttgagcc

SEQ ID NO: 16 - DNA, 134 bp, Artificial
  Mini-chromosome monomer sequence
gtcacctaggagttccatcaggtgcatccaaaataatttccgagcctatgtatgttcggtgcaaactgtgcacctatcttgcgtcaagattggcactatctctaaacggaccaaaacgagcttccacttgtgcc

SEQ ID NO: 17 - DNA, 135 bp, Artificial
  Mini-chromosome monomer sequence
gtcacctagcagttccatcaggtgtgtccaaaacaatttctgagcctatggtacgttcggcgcaaaccatgcaaatattttgagtcaagattagcaatatctccaaatggactaaaatgagcttccacttgaacc

SEQ ID NO: 18 - DNA, 131 bp, Artificial
  Mini-chromosome monomer sequence
cctaggagttacaacgggtgcgtccagaatgatttctgagctagtgatacattctgcacaaagcgtgcacctatcttgcaacaagattaacactatctgcaaacagattgaatcgagcttccacttgagcc

SEQ ID NO: 19 - DNA, 108 bp, Artificial
  Mini-chromosome monomer sequence
agaaacgagggcaggaggtgaagcggaacggaggtggccacggagcggtgttcttcggtgagctcgaccgggacttcgtcgagcgattcccacaaaggggaatcttac

SEQ ID NO: 20 - DNA, 135 bp, Artificial
  Mini-chromosome monomer sequence
gtcacctaggagttaaatcgggtgcgtccaaaactatttatgagcctatggtacgttcggcgcaaaccgtgcacctatcttgcatcaagattagctcaatctccaaacagattaaatcgcgcttccacttgagcc

SEQ ID NO: 21 - DNA, 130 bp, Artificial
  Mini-chromosome monomer sequence
tcaccttagattaccgtcgggtgtgtgcaaaacaatttatgagcctatggcacgtttggcacaaaccattatcttgcaccgacactaacactgtcaccaaacggaatgaaacgagattccacatcaccca

SEQ ID NO: 22 - DNA, 116 bp, Artificial
  Mini-chromosome monomer sequence
gggggcatcaagaacgattttgagcctctggtacgtttggcgcaaatcatgcacctatcttacatcgacagtaacattgtctccaaatagtacaaaatgacactctacacgaccca

SEQ ID NO: 23 - DNA, 137 bp, Artificial
  Mini-chromosome monomer sequence
cgtcacctaggagtataatcggctgtgtcataaatgattttttgagctgatggcacgtttggtgcaaactgtgcacctatcttgcactgaaactaacagtctccaaaaagaaaagaagtgagaatcaacattaccca

SEQ ID NO: 24 - DNA, 136 bp, Artificial
  Mini-chromosome monomer sequence
cgtcacctaggagttccatcgggtgcgtccaaaatgatttctaagcctatggtacgttcgacgcaaaccatgcacctatcttgcgtcaagatgagcactatctccgaaaggaccgaaacaagcttccacttgagcc

SEQ ID NO: 25 - DNA, 131 bp, Artificial
  Mini-chromosome monomer sequence
cgtcacctaggagatccatcgggtgcataaaaaatgatgtccgagcctatggtacttttgacacaaatcgtgcacctatcttgcatcaagatttacactatctctcaatggaccgatctttcacttgagcc

SEQ ID NO: 26 - DNA, 137 bp, Artificial
  Mini-chromosome monomer sequence
cgtcacctaggagtaccatcggctgcgtccaaaatgatttctaagccggtggtacgtttgacgcaaagtgcgcacctatcttgcaccaacactaacactatctccaaacggaaagaagtgagcttccacatgaccca

SEQ ID NO: 27 - DNA, 136 bp, Artificial
  Mini-chromosome monomer sequence
cgtcacctaggagttccatcgagtgtgtccaaaatgattaccaggatgatcagacgttccgcgccaaccatgcacctaccttgtgttaaaattagcgctatctctaaaaggaccaaaacaagcttccacttgagcc

SEQ ID NO: 28 - DNA, 117 bp, Artificial
  Mini-chromosome monomer sequence
gtcacctaggggttccatcgggagcgtccaaaataatttttgagcctatggtacgttcggaacaaaccgtgcacctatcttgcgtcaagattagcaccatctctaaatagaccaaaa

SEQ ID NO: 29 - DNA, 116 bp, Artificial
  Mini-chromosome monomer sequence
gtcgcctaggagttccatccggagcgtccaaaactatttctgaaccagtggtacgatctgtgcaaaccgtgcatcaagattagcactatctccaaatggaccaaagttgagcctcg

SEQ ID NO: 30 - DNA, 134 bp, Artificial
  Mini-chromosome monomer sequence
tgtgcataataactgttggggtccgggcaaccttaatttattgggcctcggccaaccctacctgggccggatgtatcaccgtgcacctatcttgcactaacactaacatcatctccaaacagaccgaaacgaga

SEQ ID NO: 31 - DNA, 137 bp, Artificial
  Mini-chromosome monomer sequence
cgtgacctaggagtaccatcgggtgcgtccaaaacgatttctgatcctttggtatgtttgttcaccaatcctgctctgacacaaacaacgtctccaaacggaccgaaacgagattccacatgacccacgttcggtgg

SEQ ID NO: 32 - DNA, 112 bp, Artificial
  Mini-chromosome monomer sequence
tccatcaggtgcgtccaaaactatttgtaggcctatggtacgttcggcgcaaaccatgcacctatcttgcgtcaagattagcactatttctaaatggaccaaaacgagcttc

SEQ ID NO: 33 - DNA, 135 bp, Artificial
  Mini-chromosome monomer sequence
gtcacctaggagttcaattgggtgcgtcaaaaatgattttcgagcctatggtacgtttggcacaaaccatgcacctatcttgcatcaaggttagcaccatctccaaatggaccgaaatgtacttccacatgagcc

SEQ ID NO: 34 - DNA, 132 bp, Artificial
  Mini-chromosome monomer sequence
acctaggactaaagtcgggtgtgtccaaaatgattttgagcctattgtatgtttggcgcaagtcgtgcacatatcttgcaccgaccctaacgccgtctccaaacaaatcaaaacaagattccataggaccca

SEQ ID NO: 35 - DNA, 135 bp, Artificial
  Mini-chromosome monomer sequence
tcaattaatctcgtcttgtaccaagttttaactaaagaaactcccttgatgatctttactccataagtttctaattcttgggcaacttctggatgcacttggttacactgtcaactcaaaacaaaaacctaggac

SEQ ID NO: 36 - DNA, 136 bp, Artificial
  Mini-chromosome monomer sequence
cgtctcctaggacttctatcgggtgcatccgaaatgatttctgagtcgacagtacattcagcgatgtgcgacgaccttgtgttaagattaacactatctctaaatgaactgaaatgagcttctagttgagccctat

SEQ ID NO: 37 - DNA, 128 bp, Artificial
  Mini-chromosome monomer sequence
cgtcatctaggagttccatcgggtgagtccaaaactatttctaagcctatggtacgttcgacgcaaaccatgcacctatcttgcatcaagattagcactatctccattggaccaaaatgagcttccag

SEQ ID NO: 38 - DNA, 135 bp, Artificial
  Mini-chromosome monomer sequence
cgtcaccaaggagttccattgggtgtgttcaaaacaatttctgatcctagggtacgtttggcacaaactgcgcacctatcttgtatcaagattagccctatctgcaaacagactgaatcaagcttccacttgagc

SEQ ID NO: 39 - DNA, 138 bp, Artificial
  Mini-chromosome monomer sequence
cgtcacctaggagttcaatgatgtgtgtccaaaacgattttctaagcctatggtttgttcagcccaaaccaagcacctactgtgcaccgacacaaataatatctccaaacagaccaaaatgagattccacatgatcca

SEQ ID NO: 40 - DNA, 133 bp, Artificial
  Mini-chromosome monomer sequence
taggaggcttgtaatgctccggggcttttgcctaggtttgaagcacaatgtcagttagcttcatcttcaggacatgatcaaccatcatctttatctcctagacttggtcactgttacaccatcttgtgatcgg

SEQ ID NO: 41 - DNA, 121 bp, Artificial
  Mini-chromosome monomer sequence
cgtcacctaggagtaccatcaggtgtgtccaaaatggtttctgagtctatggtatgtttggcacaaaccgtcaagattagcattatctcctaacggaatgaaatgagcatgcacttgagcc

SEQ ID NO: 42 - DNA, 113 bp, Artificial
  Mini-chromosome monomer sequence
gtcacctaggagttccatcgagtgtgtccgaaacgatttccgagccttttggtatgttcggcgcaaaacgtgcacctatcttgcatcaagattagcactatctccaaacggac

SEQ ID NO: 43 - DNA, 131 bp, Artificial
  Mini-chromosome monomer sequence
cgtcaccttggagtaccatttggtttgtgtaaaacgatttatgagctcatggcacatttgacgcaaaccaagcaccacttgtcaagattagcactatcggcaaacacaccgaacagatctccaccagaccc

SEQ ID NO: 44 - DNA, 134 bp, Artificial
  Mini-chromosome monomer sequence
tcacctaggagttccatcgggtgcctccaatactatttctgagcctacggtacgttcgatgcaaaccgtgcaccaatcttgcatcaagagtagcactatctccaaacggaccaaagagagctcccacttgagcc

SEQ ID NO: 45 - DNA, 124 bp, Artificial
  Mini-chromosome monomer sequence
taggagtatcatgggtgcgtctaaaatgatttatgagcctatggtacgtttgacacaaaccaagcatttgcaccgacgctaacactgtctcaatgtagatcaaaacaagattccacatgaccca

SEQ ID NO: 46 - DNA, 130 bp, Artificial
  Mini-chromosome monomer sequence
ccttttctatcaacggtgctgcgtcaccttattttgggccgatggcccatgtatcaagttgggtccattagggacgcgtcctagggttggagaatgactcaaacacccttgtggtcgtcctcccatctac

SEQ ID NO: 47 - DNA, 117 bp, Artificial
  Mini-chromosome monomer sequence
cgtcacctaggagttccatcgggtgccgctaatactatttctgagccaacggtacgttcgacgcaaatcgtgcaccaatcttgcaccaagtgtagcactgtctccaaacggatcgaa

SEQ ID NO: 48 - DNA, 133 bp, Artificial
  Mini-chromosome monomer sequence
cgtctcctaggagtaccatcaagtgcgtccaaatcgatttcagggtgtgttacctgattggtacgaaccgtccaactatcttgcaccgacactaacactgtctccaaatagactgaaacaagattccacatga

SEQ ID NO: 49 - DNA, 114 bp, Artificial
  Mini-chromosome monomer sequence
catcggatgcatctaaaccaatttccgagcctatggtatgttctgcgcaacccgtgcacctcgtcaagatcagcactatctccaaacggaccaaaacgagcttccacttgagcc

SEQ ID NO: 50 - DNA, 133 bp, Artificial
  Mini-chromosome monomer sequence
cgtcacctaggagtaccatcgggtgcgtccaaattgatttcagggcgtatggtacgattggtgcgaaccgtccaactatcttgcaccgacactaacactatctccaaatagactgaaacaagattccgcatga

SEQ ID NO: 51 - DNA, 136 bp, Artificial
  Mini-chromosome monomer sequence
cgacacctaggagttccattgggtgcgtccaaaatgattctcgagcctctggtacgttctgcgcaaaccgtgcacctatcttgcagaaagattagcaccatctccaaatggaacaaaacgagcttccacttgagcc

SEQ ID NO: 52 - DNA, 120 bp, Artificial
  Mini-chromosome monomer sequence
ccatggggtgtgtccaaaatgatttccaatcctatggtatgttttgcgtaatccgtgcacctatcttgcatcaagtttagcaccatctccaatcggaccgtaacgagcttccacttgagc

SEQ ID NO: 53 - DNA, 130 bp, Artificial
  Mini-chromosome monomer sequence
tcacctaggagtaccatcgggtgcatacaaaataatttccaagcctatggtaccttcggcgcaaaccatgcattatcttgcgctaagactaacactatctctaaacagaccgaaacaagctttcacttga

SEQ ID NO: 54 - DNA, 136 bp, Artificial
  Mini-chromosome monomer sequence
cgtcacctaggagtaccatcggctgtgcctaaaatgatttccacgcccatggtacattcggcccaaaccgtgcacctatcttgcggcgagattaacactatctccaaatggaccgaaacgagttccacttgaccca

SEQ ID NO: 55 - DNA, 113 bp, Artificial
  Mini-chromosome monomer sequence
agctccagggcatgtgcggaaagtgatttccatgcccgtggtagatttgtcgcaaaccgtgcacctatcttgtgattctccaaacggagcgaaatgagcttccacttgaccca

SEQ ID NO: 56 - DNA, 133 bp, Artificial
  Mini-chromosome monomer sequence
cgtcacctaggagtaccatcgggtgcgtccaaaattatttctaagcctatggtacgtttggcgcaaaccgtgcacctatcttgcgccgagactaacactatctccaaacggacccaaacaagcttccacttga

SEQ ID NO: 57 - DNA, 121 bp, Artificial
  Mini-chromosome monomer sequence
cgtcacctaggagtaccattaggtgcattcaaaataatttccaagcttatggtacgttacctatcttgcaccaaggctaacactatctctaaacggatcgaaatgagattcccttgaccca

SEQ ID NO: 58 - DNA, 111 bp, Artificial
  Mini-chromosome monomer sequence
gtccataacgatttctgagcctatggtctgtttgacgcaaaccgtgcacctatcttgcgtcgagattaacactatctccaaatagactgaaacaaacttccatttgaccca

SEQ ID NO: 59 - DNA, 134 bp, Artificial
  Mini-chromosome monomer sequence
tcacctaggagttccatcgggtgtgcccaaaatgattccaagcctattgtacattcgacgcaaaccgtgcacctatcttgcatgtcgattaacactatttccaaacagaccaaaacgagcttccatttgaccca

SEQ ID NO: 60 - DNA, 128 bp, Artificial
  Mini-chromosome monomer sequence
tcacctaggagttccattgggtgtgcccaaaatgattttcaagcctattagacattcaatgtgtgaatggtgcacctatcgtgtgtaacactatctccaatggccgatatgagcatctacttgaccca

SEQ ID NO: 61 - DNA, 136 bp, Artificial
  Mini-chromosome monomer sequence
tatttctaagttctaagttcttatttagtttttactttattctatttacttgaactccgcttgtatttgagtgctctagttataatatttggctagagtagtaaaccctagcgtagatgtggtgtctaggctatag

SEQ ID NO: 62 - DNA, 137 bp, Artificial
  Mini-chromosome monomer sequence
cgccacctacgagttccatcagatgtgcccaaaatgatttccaagctcgttacacaatcaacacaatttgggcacctatcatgtatcgagattagcactaccacaatagagactgaaacaagcttccacttaaccca

SEQ ID NO: 63 - DNA, 137 bp, Artificial
  Mini-chromosome monomer sequence
cgtcacctagaagttccatcgggtgcgtccaaaatgatttctgagcccgtggtaagttcggcgcaaaccgtgcaccaatcttgcgtcaagattagcactatctccaaacagaccgaatcgaacttccacttgagcca

SEQ ID NO: 64 - DNA, 135 bp, Artificial
  Mini-chromosome monomer sequence
cgacacataggtattctaccgagtgtgtctaaaacaattttcgagcatgtggtatgttcggtgcatatcgtgcaccttcctgtgtcaagattagcactatctccaaacggaccgaaatgagcatccacttgtgcc

SEQ ID NO: 65 - DNA, 107 bp, Artificial
  Mini-chromosome monomer sequence
cgtcatccagaactatcaatggccgtgtccaaaatgatttctaagcccttggtatgtttggcgcaaacgatgcacctatcctacaccgacactaacactaacactaa

SEQ ID NO: 66 - DNA, 135 bp, Artificial
  Mini-chromosome monomer sequence
gtcacctaggagtaccatcgggtgcgtccagaatgatttctgagcctatggtacatttggagcaacccatgcacctatcttgcaccgacactaacactatctccaaacggaccgaaacgagattccaaatgaccc

SEQ ID NO: 67 - DNA, 117 bp, Artificial
  Mini-chromosome monomer sequence
cgtcacctaggagttccatcgggtgcatccaaaatgatttccgagcctatggtacgttcgacgcaaaccgtgcacctagcttgtgtcaagatgagcactatctccaaacggaccgaa

SEQ ID NO: 68 - DNA, 136 bp, Artificial
  Mini-chromosome monomer sequence
tgcgtcccaaactacttgtgagcctatggtacgttcagtgcaaaccgtgcacctatcttgtgtcaagattggcactatctccaaacggaccaaacagagctccaccagaccctcgtcacttaggagtaccatcggg

SEQ ID NO: 69 - DNA, 135 bp, Artificial
  Mini-chromosome monomer sequence
tgcctccaatactatttccgagcatacggtacatttgacgcaaaccgtgcaccaatcgtgcatcaagagtagcactatctccaaacggaccgaaccaagcttccacttgagcctcgtcaccaaggagtcccatcg

SEQ ID NO: 70 - DNA, 135 bp, Artificial
  Mini-chromosome monomer sequence
tgcctccaatactatttctgagcctatggtatgtttgacgcaaaccgtgcaccaatcgtgcatcaagagtagcactatctccaaacagaccgaaccaagcttccacttgagcctcgtcaccaaggaatcccatcg

SEQ ID NO: 71 - DNA, 136 bp, Artificial
  Mini-chromosome monomer sequence
tgcgtcaaaaactatttgtgagcctatggtacgtctagtgcaaaccgtgcacctatcttgtgtcaagattagcactatctccaaacggaccgaacagagctccaccagacccacgtcacttaggagtaccatcggg

SEQ ID NO: 72 - DNA, 134 bp, Artificial
  Mini-chromosome monomer sequence
tgcgtctaaattgatttatgaccctatggtacgtttgacgcaaattgagcacctatcatgcaccgacgctaacactgtctcaaaatagatcgaaacgagatcccacaagacccacgtcaactaggagttccatc

SEQ ID NO: 73 - DNA, 137 bp, Artificial
  Consensus satellite repeat sequence
ccyagsagttccatcgggtgcgtccaaaaygatttyygagcctatggtacgttygrcgcaaaccgtgcacctatcttgcatcaagabtagcactatctccaaacggaccgaaacgagcttccacttgagccacgtca

SEQ ID NO: 74 - DNA, 3465 bp, Saccharum officinarum
gggaagtaccagggacgaagagctagcgcaaggactaatgtttctgtagggaaagctagttcatggcagcagcgcacgtctacaactccgtctacatgtactcctacgccatcatctagtgacaagactcgagctgcccccaccaattcagtmrcgaagacagcgcaaaagccggctgcgagtacttcatccgtggcatcgacmggtagaacaagcaacatacaatgtcatcggtgcaagggatatggccatatgatgcgtgactgcccaaacaagcgtgttatgattgtcaaagatgatggtgagtgttcatctgctagtgattttgatgaagatacacttgcattgcttgcgctgactatgcaggtagtgaggaacacatagaagaacacatyaatgcaggtgacgcggagcactatgagagcttgattgtacagcgagtgcttagtgtacaaatggagaaggyagagcaaaatcagcgacacactttattccaaacaaagtgtgtcatcaaggagcgttcgtgccgcatgatcattgatggaggtagctgcaacaacttggcaagtagcgacatggtacagaagctcgcctcaccaccaaacacacccccatccctactacatccaatggctgaacaacagtggtaaggcaaaggtaacagacttgtgcgaattgattttgccatcggatcatacaaagatattgttgaatgtgatgttgtgcctatgcaagcttgkaatattctgctaggtagaccatggcaatttgatagagattcgatgcatcatggtagatcaaatcaatattcgtttctataccatgatcgaaaaattgtgttgcatcctatgtctcctgaagctattatgcaatctgatgttactaaggctgcaaaagcaaagagcgagagcaataaaaatgacaaatctgttgttgatgacaaagatgagataaaattgaaaggacgttgtatgcttgctacaaaatctgatattaatgagttcaatgcatccacctctgttgcttatgctttggtatgcaaggatgctttgatttcatttgaggatatgcaacgttctttgccccctgtgtgctaacattttgcaggagtattctgatgtgttccccattgagataccagcggggctgccaccaatacgtgggattgagcaccaaattgatcttatccctggagcatctttgccaaaccgtgcgccatacaggaccaatccggaagaagcgaaggaaattcagcgacaagtgcaagaactactagacaaaggttatgtgcgtcagtctcttagtccttgtgctgttccagtgattttagtgcctaagaaagatggaacatggcgcatgtgtgttgattgtagggctattaataatatcaccattcgatatcgacccctattccacgattagatgatatgcttgatgaattgagtggtgctgttgtgttttcaaaggttgatttgcgtagtgggtaccaccagattcgtatgaaattgggagatgaatggaaaactgctttcaaaactaagttcggtctatatgagtggttagtcatgccttttgggttaactaatgcacctagtactttcatgagattaatgaacgaggttttgcgtgctttcattgggaaatttgttgtcgtctactttgatgatatactgatttatagcaaatccatggatgaacaccttgatcacttacgtgctgttttcaatgctctacgtgatgcacgtttgtttggtaaccttgagaagtgcactttctgcaccaatcgagtatcgtttcttggctatgttgtcactccacagggaattgaggttgataaagccaaggtggaagctatacaaggatggcctgtacccatgacggtcacacaagtgcggagtttcctaggacttgctggtttctatcgccgtttygtgaaggacttcagcaccattgctgcaccattgaatgagcttacaaagaagggagtgcccttttcttggggcaaagcacaagagcacgccttccatgtgctgaaagataagttaacacatgcacctctactccaactccctgattttaataagactttcgagcttgaatgtgatgcgagtggaattggattgggtggtgttttgttgcaagaaggcaaacctgttgcatactttagtgagaaattgagcgggcctattcttaattattctacatatgataaggaattatatgctctagtgcggacattagaaacatggcagcattatttgtggcccaaagaatttgttatacattctgatcatgaatctttgaagcatattcgtagtcaaggaaaactgaatcgtagacatgcaaatgggttgaatttattgaatcttttccttatgttatcaaacacaagaaagggaaagataatatcattgcagatgcattgtctaggagatatactttgctgaatcaacttgattacagattttgggttaaaacaattaaagacgatatgctcatgatgctgattttaaagaagtgctgctgcattgtaaagatgggaaaacgtggaacaaattcatcgtcaatgatgggtttgtgtttagagctaacaagctatgcattccagctagctccgttcgcttgttgttgctgcaggaagcgcatggaggtggcttgatggggcattttggagcgaagaagacggaggacattcttgctggtcatttcttttggccaaagatgcgacgagacgtggagagatttgttgctcgctgcacaacatgtcaaaaggctaatcccggttgaatccacacggtttgtatatgcctcttcctgttcctagtgctccttgggaggatatttctatggattttgtgttgggattgccacgaactaggaaagggcgagatagtgtttttgtggttgtggatcgattctctaaaatggcacatttcataccatgtcataaaactgatgatgctacaaatattgctgatttgttctttcgagaagttgttcgcttgcatggtgtgccaaatacaattgtttctgatcgtgatgctaaatttcttagccatttttggagaactttatgggctaaattggggactaagcttttmttttccaccacttgtcacccccaaactgatggtcaaactgagttgtgaaagaacttgtctacatgtaggctttttaaagaagaatattaagattgggaagattgtttgcctcatttgagtttcttaaatcgtcktgcattccacgaagatgtgtccytttgagtgtytatgggttttccacggctccattgatttaatgcmcwttgcamstttgraaagctasaattttgatgctaagcaactgctaattgatggywaacatmcmtgaaaaccacyaaagaaaacatasagcgcatgaatgctaartacaaatttgykggagataarggtagaargtaattgcttctggacccggggaattggt

SEQ ID NO: 75 - DNA, 25 bp, Artificial
  PCR primer
gtcacccagcagttccatcgggtgc

SEQ ID NO: 76 - DNA, 24 bp, Artificial
  PCR primer
actgctgggtgacgtggctcaagt

SEQ ID NO: 77 - DNA, 22 bp, Artificial
  PCR primer
gggaagtacagggacgaagagc

SEQ ID NO: 78 - DNA, 20 bp, Artificial
  PCR primer
actaacaatgcacgggaagg

SEQ ID NO: 79 - DNA, 20 bp, Artificial
  PCR primer
gtaggccatggcagtttgat

SEQ ID NO: 80 - DNA, 20 bp, Artificial
  PCR primer
aacacaccacccaatccaat

SEQ ID NO: 81 - DNA, 23 bp, Artificial
  PCR primer
ccaaacaagcgtgttatgattgt

SEQ ID NO: 82 - DNA, 24 bp, Artificial
  PCR primer
aggttatgtgcgtcagtctcttag

SEQ ID NO: 83 - DNA, 23 bp, Artificial
  PCR primer
ggcaaacctgttgcatactttag

SEQ ID NO: 84 - DNA, 23 bp, Artificial
  PCR primer
accatgtcataaaactgatgatg

SEQ ID NO: 85 - DNA, 22 bp, Artificial
  PCR primer
tgcaaccaaaccaaatcaccag

SEQ ID NO: 86 - DNA, 20 bp, Artificial
  PCR primer
caagcgaacaatctcacgaa

SEQ ID NO: 87 - DNA, 20 bp, Artificial
  PCR primer
aaatcatcatcgtgcgcata

SEQ ID NO: 88 - DNA, 20 bp, Artificial
  PCR primer
aacacaccacccaatccaat

SEQ ID NO: 89 - DNA, 19 bp, Artificial
  PCR primer
gaacgctccttgatgacac

SEQ ID NO: 90 - DNA, 22 bp, Artificial
  PCR primer
gtacccactacgcaaatcaacc

SEQ ID NO: 91 - DNA, 23 bp, Artificial
  PCR primer
caacttcagtttgaccatcagtt

SEQ ID NO: 92 - DNA, 20 bp, Artificial
  PCR primer
aagtcggcagagagcaacat

SEQ ID NO: 93 - DNA, 20 bp, Artificial
  PCR primer
cagatgcaaacccaacacac

SEQ ID NO: 94 - DNA, 24 bp, Artificial
  PCR primer
cgattttctgggtttgatcgttag

SEQ ID NO: 95 - DNA, 20 bp, Artificial
  PCR primer
cattgtgggcgttgtagttg

SEQ ID NO: 96 - DNA, 20 bp, Artificial
  PCR primer
gttgtggttggtgctttcct

SEQ ID NO: 97 - DNA, 20 bp, Artificial
  PCR primer
ccactttgacgccgtttatt

SEQ ID NO: 98 - DNA, 24 bp, Artificial
  PCR primer
cgattttctgggtttgatcgttag

SEQ ID NO: 99 - DNA, 21 bp, Artificial
  PCR primer
gaactcgtcaagaaggcgata

SEQ ID NO: 100 - DNA, 24 bp, Artificial
  PCR primer
cgattttctgggtttgatcgttag

SEQ ID NO: 101 - DNA, 18 bp, Artificial
  PCR primer
tcagggcaatgcagatcc

SEQ ID NO: 102 - DNA, 18 bp, Artificial
  PCR primer sequence
ccagcctcatggccaaag

SEQ ID NO: 103 - DNA, 20 bp, Artificial
  PCR primer sequence
ccttcttggcggcttatctg

SEQ ID NO: 104 - DNA, 18 bp, Artificial
  PCR primer sequence
aaagcatatagcacaaca

SEQ ID NO: 105 - DNA, 22 bp, Artificial
  PCR primer sequence
tatgttatgtgtgtcttgtata

SEQ ID NO: 106 - DNA, 19 bp, Artificial
  PCR primer sequence
cataagtccatcagttcaa

SEQ ID NO: 107 - DNA, 18 bp, Artificial
  PCR primer sequence
ccaaggcatttattctca

SEQ ID NO: 108 - DNA, 26 bp, Artificial
  Adh1 probe sequence
cttaggggcagactcccgtgttccct

SEQ ID NO: 109 - DNA, 21 bp, Artificial
  Yat1 probe sequence
agcatcagcatcagcactaga

SEQ ID NO: 110 - DNA, 18 bp, Artificial
  RedF probe sequence
tcgcaaccgcatcagact

(19) United States
(12) Patent Application Publication
(10) Pub. No.: US 2014/0047583 A1
(43) Pub. Date: Feb. 13, 2014

(54) CENTROMERE SEQUENCES DERIVED FROM SUGAR CANE AND MINICHROMOSOMES COMPRISING THE SAME

(71) Applicants: Song Luo, Chicago, IL (US); Gregory P. Copenhaver, Chapel Hill, NC (US); Dephne Preuss, Chicago, IL (US)

(72) Inventors: Song Luo, Chicago, IL (US); Gregory P. Copenhaver, Chapel Hill, NC (US); Dephne Preuss, Chicago, IL (US)

(73) Assignee: CHROMATIN, INC, Chicago, IL (US)

(21) Appl. No.: 13/981,841

(22) PCT Filed: Jan. 13, 2012
(86) PCT No.: PCT/US12/21211

§ 371 Date: Oct. 29, 2013

Related U.S. Application Data
(60) Provisional application No. 61/436,484, filed on Jan. 26, 2011.

(51) Int. Cl. C12N 15/82

(52) U.S. Cl. CPC C12N 15/82
USPC 800/298; 435/320.1; 435/419

(57) ABSTRACT

The invention is generally related to sugar cane mini-chromosomes containing sugar cane centromere sequences. In addition, the invention provides for methods of generating sugar cane plants transformed with these sugar cane mini-chromosomes. Sugar cane mini-chromosomes with novel compositions and structures are used to transform sugar cane cells which are in turn used to generate sugar cane plants. Methods for generating sugar cane plants include methods for delivering the sugar cane mini-chromosomes into sugar cane cell to transform the cell, methods for selecting the transformed cell, and methods for isolating sugar cane plants transformed with the sugarcane mini-chromosome.

### RELATED APPLICATION INFORMATION

This application claims the benefit of U.S. Provisional Application No. 61/436, 484 filed Jan. 26, 2011, the disclosure of which is incorporated by reference herein in its entirety.

### FIELD OF THE INVENTION

The present invention generally relates to sugar cane satellite repeat sequences, sugar cane centromeres and mini-chromosomes containing sugar cane satellite repeat or centromere sequences as well as sugar cane cells and plants comprising the same.

### BACKGROUND OF THE INVENTION

Two general approaches are used for introduction of new heritable genetic information (“transformation”) into cells. One approach is to introduce the new genetic information as part of another DNA molecule, which can optionally be maintained as an independent unit (e.g., an episome referred to as a “mini-chromosome”) apart from the host chromosomal DNA molecule(s). Episomal vectors can contain all the DNA sequence elements for DNA replication and maintenance of the vector within the cell. Many episomal vectors are available for use in bacterial cells (for example, see Maniatis et al., “Molecular Cloning: a Laboratory Manual,” Cold Spring Harbor Laboratory, Cold Spring Harbor, N.Y. 1982). However, only a few episomal vectors that function in higher eukaryotic cells have been developed. Higher eukaryotic episomal vectors are primarily based on naturally occurring viruses. In higher plant systems gemini viruses are double-stranded DNA viruses that replicate through a double-stranded intermediate upon which an episomal vector can be based. Although an episomal plant vector based on the Cauliflower Mosaic Virus has been developed, its capacity to carry new genetic information is also limited (Brisson et al., Nature, 310:511, 1984).

The other general method of genetic transformation involves integration of introduced DNA sequences into the recipient cell's chromosomes, permitting the new information to be replicated and partitioned to the cell's progeny as a part of the natural chromosomes. The introduced DNA can usually be broken and joined together in various combinations before it is integrated at random sites into the cell's chromosome (see, for example Wigler et al., Cell, 11:223, 1977). Common problems with this procedure are the rearrangement of introduced DNA sequences and unpredictable levels of expression due to the location of the transgene integration site in the host genome or so called “position effect variegation” (Shingo et al., Mol. Cell. Biol., 6:1787, 1986). Further, unlike episomal DNA, integrated DNA generally cannot be precisely removed. A more refined form of integrative transformation can be achieved by exploiting naturally occurring viruses that integrate into the host's chromosomes as part of their life cycle, such as retroviruses (see Chepko et al., Cell, 37:1053, 1984).

One attractive method for transformation employs the use of a mini-chromosome. Mini-chromosomes are nucleic acid molecules that can optionally be episomal and exist autonomously from the native chromosomes of the host genome. They can be linear or circular DNA molecules that are comprised of cis-acting nucleic acid sequence elements that provide replication and partitioning activities (see Murray et al., Nature, 305:189-193, 1983). Exemplary elements that may be incorporated into the mini-chromosome include: (1) an origin of replication, which is a sites for initiation of DNA replication (e.g., an origin of replication from sugar cane genomic DNA), (2) a centromere (site of kinetochore assembly and responsible for proper distribution of replicated chromosomes into daughter cells at mitosis or meiosis), (3) if the mini-chromosome is linear, it will generally contain telomeres (specialized DNA structures at the ends of linear chromosomes that function to stabilize the ends and facilitate the complete replication of the extreme termini of the DNA molecule), and (4) a chromatin organizing sequence. It is well documented that centromere function to facilitate stable chromosomal inheritance in almost all eukaryotic organisms. The centromere accomplishes this by attaching, via centromere binding proteins, to the spindle fibers during mitosis and meiosis, thus ensuring proper gene segregation during cell divisions.

Mini-chromosomes have been engineered using one of two approaches. The first approach identifies and assembles the desired chromosomal elements into an artificial construct. This approach has been described as “bottom-up” and typically involves the use of a heterologous system (e.g., bacterial or fungal) to perform the various cloning steps necessary to assemble the mini-chromosome. The second approach derives the mini-chromosome from existing chromosomes through chromosome fragmentation and, optionally, subsequent addition of desired elements including transgenes. For example, an existing chromosome can be induced to undergo breakage events that result in chromosomal fragments. Minimal fragments that possess the elements for replication and segregation during cell division (e.g., centromere, origins of replication and/or telomeres) can be identified. These derived mini-chromosomes can then be used as targets for further manipulation including the addition of one or more transgenes. This approach has been described as “top-down” and generally does not require the use of a heterologous system (e.g., bacterial or fungal) since it does not require in vitro-based cloning steps. Mini-chromosomes of this type are referred to in this application as “recombinant chromosomes”. The chromosomal elements for construction of mini-chromosomes have been characterized in lower eukaryotic species, and more recently in mouse and human Autonomous replication sequences (ARSs) have been isolated from unicellular fungi, including Saccharomyces cerevisiae (brewer's yeast) and Schizosaccharomyces pombe. An ARS behaves like an origin of replication allowing DNA molecules that contain the ARS to be replicated in concert with the rest of the genome after introduction into the cell nuclei of these fungi. DNA molecules containing these sequences replicate, but in the absence of a centromere they are not partitioned into daughter cells in a controlled fashion that ensures efficient chromosome inheritance.

Mini-chromosomes have been constructed in yeast using cloned chromosomal elements (see Murray et al., Nature, 305:189-193, 1983). None of the components identified in unicellular organisms, however, have been shown to function in higher eukaryotic systems. For example, a yeast centromere sequence will not confer stable inheritance upon vectors transformed into higher eukaryotes.

In contrast to the detailed studies done in yeast, less is known about the molecular structure of functional centromeric DNA of higher eukaryotes. Ultrastructural studies indicate that higher eukaryotic kinetochores, which are specialized complexes of proteins that form on the centromere during late prophase, are large structures (mammalian kinetochore plates are approximately 0.3 μm in diameter) that possess multiple microtubule attachment sites.

There exists a need for cloned centromeres from sugar cane, which would represent a first step in the production of sugar cane mini-chromosomes. There further exists a need for sugar cane cells, plants, seeds and progeny containing mini-chromosomes capable of carrying one or more genes and/or genetic elements.

### SUMMARY OF THE INVENTION

The invention is based, in part, on the identification of polynucleotides comprising, consisting essentially of, or consisting of sugar cane repeated nucleotide sequences (e.g., from sugar cane genomic DNA) or sequences substantially identical thereto (e.g., at least 70%) and isolated nucleic acids comprising the polynucleotides of the invention or an array comprising two or more copies of a polynucleotide of the invention. The invention also provides centromere sequences comprising sugar cane genomic DNA or sugar cane satellite repeat sequences and mini-chromosomes comprising the such centromere sequences.

Accordingly, in one aspect, the present invention provides sugar cane mini-chromosomes comprising a sugar cane centromere having one or more repeated nucleotide sequences, described in further detail herein. In some embodiments, such mini-chromosomes comprise a centromere comprising one or more repeated nucleotide sequences derived from sugar cane, including those isolated from sugar cane genomic DNA.

In another aspect, the invention provides modified or “adchromosomal” sugar cane plants, comprising a mini-chromosome of the invention, e.g., a sugar cane plant comprising a functional, stable, autonomous mini-chromosome.

The invention also provides for sugar cane mini-chromosomes comprising a centromere, wherein the centromere comprises at least two copies of a repeated nucleotide sequence(s), and optionally wherein the centromere confers the ability to segregate to daughter cells. The repeated nucleotide sequence(s) may be sugar cane satellite sequences such as those represented by SEQ ID NOS: 1-72 and sequences that are substantially identical to such sequences and/or hybridize to such sequences under stringent hybridization conditions (e.g., comprising hybridization at 65° C. and washing three times for 15 minutes with 0.25×SSC, 0.1% SDS at 65° C. or hybridization in 0.02 M to 0.15 M NaCl at temperatures of about 50° C. to 70° C.). The repeated nucleotide sequence(s) may be oriented in a head to tail, tail to head and/or head to tail orientation. The sugar cane centromere or mini-chromosome may further comprise other repeated nucleotide sequences such as one or more sequences derived from the sugar cane retrotransposon sequence CRS (e.g., SEQ ID NO:74), including fragments and variants thereof.

The invention also provides cells comprising a polynucleotide, nucleic acid, vector, sugar cane centromere, and/or sugar cane mini-chromosome of the invention. In embodiments, the cell is an isolated cell. In other representative embodiments, the cell is a sugar cane cell.

Accordingly, as one aspect, the invention provides a polynucleotide comprising, consisting essentially of, or consisting of: (a) the nucleotide sequence of any of SEQ ID NOS: 1-72; (b) a nucleotide sequence that is substantially identical to the nucleotide sequence of any of SEQ ID NOS: 1-72, optionally wherein the nucleotide sequence is functional as a sugar cane plant centromere (e.g., confers the ability to segregate to a daughter cell); and/or (c) a nucleotide sequence that hybridizes to the nucleotide sequence of any of SEQ ID NOS: 1-72 under stringent conditions (e.g., comprising hybridization at 65° C. and washing three times for 15 minutes with 0.25×SSC, 0.1% SDS at 65° C. or hybridization in 0.02 M to 0.15 M NaCl at temperatures of about 50° C. to 70° C.), optionally wherein the nucleotide sequence is functional as a sugar cane plant centromere.

As another aspect, the invention provides a nucleic acid comprising an array comprising at least about two, at least about 5, at least about ten, at least about 25, at least about 50, at least about 100, at least about 150, at least about 200, at least about 250, at least about 300, at least about 350, at least about 400, at least about 450, at least about 500, at least about 600, at least about 700, at least about 800, at least about 900, at least about 1000, from about 2 to about 25, 50, 100, 150, 200, 250, 300, 350, 400, 450, 500, 600, 700, 800, 900 or 1000, from about 5 to about 25, 50, 100, 150, 200, 250, 300, 350, 400, 450, 500, 600, 700, 800, 900 or 1000, from about 10 to about 50, 100, 150, 200, 250, 300, 400, 500, 600, 700, 800, 900 or 1000, or from about 25 to about 50, 100, 150, 200, 250, 300, 350, 400, 450, 500, 600, 700, 800, 900 or 1000 copies of a polynucleotide of the invention.

In illustrative embodiments, the invention provides a nucleic acid comprising an array comprising, consisting essentially of, or consisting of from about 42 to about 540 copies of a polynucleotide of the invention. In other particular embodiments, n is about 483 (e.g., satellite repeat sequences based on SEQ ID NO:1), about 242 (e.g., satellite repeat sequences based on SEQ ID NO:2), about 621 (e.g., satellite sequences repeat based on SEQ ID NO:3), about 102 (e.g., satellite repeat sequences based on SEQ ID NO:4), about 346 (e.g., satellite repeat sequences based on SEQ ID NO:5), about 540 (e.g., satellite repeat sequences based on SEQ ID NO:6), about 339 (e.g., satellite repeat sequences based on SEQ ID NO:7), about 42 (e.g., satellite repeat sequences based on SEQ ID NO:8), about 276 (e.g., satellite repeat sequences based on SEQ ID NO:9), about 469 (e.g., satellite repeat sequences based on SEQ ID NO:10), about 408 (e.g., satellite repeat sequences based on SEQ ID NO:11) or about 99 (e.g., satellite repeat sequences based on SEQ ID NO:12).

As used herein the term “satellite repeat content based on SEQ ID NO: X” or “satellite repeat sequence that is based on SEQ ID NO: X” indicates the nucleotide sequence of SEQ ID NO: X and/or a nucleotide sequence that is substantially similar to SEQ ID NO: X and/or a sequence that hybridizes to SEQ ID NO: X under stringent hybridization conditions. In embodiments of the invention, the term “sugar cane satellite repeat sequence” or “sugar cane satellite repeat content” refers to any of SEQ ID NOS: 1-72 and/or a nucleotide sequence that is substantially similar to any of SEQ ID NOS: 1-72 and/or a sequence that hybridizes to any of SEQ ID NOS: 1-72 under stringent hybridization conditions.

In representative embodiments, the array is from about 1 to about 25, 50, 75, 100, 125, 150, 200 or 250 kb in length, optionally from about 5, 10 or 15 to about 50, 75, 100, 125 or 150 kb in length. In other embodiments, the array is from about 21 kb to about 137 kb in length. To illustrate, the array can be about 44 kb, 69 kb, 83 kb, 85 kb, 96 kb, 103 kb, 108 kb, 132 kb or 137 kb in length. Alternatively, the array can be from about 6 kb to about 85 kb in length, for example, about 6 kb, 14 kb, 33 kb, 37 kb, 46 kb, 47 kb, 55 kb, 64 kb, 66 kb, 73 kb or 85 kb in length. In other particular embodiments, the array can be about 6 kb to 101 kb in length, for example, 6 kb, 21 kb, 38 kb, 45 kb, 47 kb, 51 kb, 56 kb, 68 kb, 70 kb, 74 kb or 101 kb in length. In exemplary embodiments, the nucleic acid is functional as a sugar cane plant centromere.

As still another aspect, the invention provides a polynucleotide comprising, consisting essentially of, or consisting of (i) the nucleotide sequence of a minichromosome as described herein, (ii) the nucleotide sequence of a minichromosome as described herein excluding any exogenous nucleic acid sequence(s), or (iii) the repeat sequences of a minichromosome as described herein, the minichromosome including without limitation: minichromosome MC CHROM5802; minichromosome MC CHROM5809; minichromosome MC CHROM5810; minichromosome MC CHROM5814; minichromosome MC CHROM5817; minichromosome MC CHROM5819; minichromosome MC CHROM5820; minichromosome MC CHROM5823; minichromosome MC CHROM5829; minichromosome MC CHROM5834; minichromosome MC CHROM5839; minichromosome MC CHROM5824; minichromosome MC CHROM6000; minichromosome MC CHROM6030; minichromosome MC CHROM6018; minichromosome MC CHROM6032; minichromosome MC CHROM6003; minichromosome MC CHROM6029; minichromosome MC CHROM6022; minichromosome MC CHROM6006; minichromosome MC CHROM6028; minichromosome MC CHROM6027; minichromosome MC CHROM6020; and minichromosome MC CHROM6023.

The invention further encompasses a sugar cane centromere comprising a polynucleotide or nucleic acid of the invention. The invention further provides a sugar cane centromere from sugar cane genomic DNA, optionally comprising an array comprising one or more copies (as described herein) of a polynucleotide of the invention. In embodiments of the invention, the centromere confers the ability to segregate to daughter cells.

In representative embodiments, the polynucleotides, nucleic acids, sugar cane centromeres or mini-chromosomes of the invention are isolated polynucleotides, nucleic acids, sugar cane centromeres or mini-chromosomes, respectively.

Also provided by the invention is a sugar cane mini-chromosome comprising a polynucleotide, nucleic acid or centromere of the invention.

In representative embodiments, the sugar cane mini-chromosome comprises a plurality of restriction sites for insertion of an exogenous nucleic acid(s).

In embodiments of the invention, the sugar cane mini-chromosome further comprises an exogenous nucleic acid (e.g., at least about three exogenous nucleic acids), at least one of which may optionally be linked to a heterologous regulatory sequence functional in sugar cane plant cells.

In further representative embodiments, the sugar cane mini-chromosomes of the invention exhibit a mitotic segregation efficiency in sugar cane plant cells of at least about 60%, 70%, 80%, 85%, 90%, 95% or more.

Also encompassed by the present invention is a vector comprising a polynucleotide, nucleic acid, sugar cane centromere, or sugar cane mini-chromosome of the invention.

As still another aspect, the invention provides a cell comprising a polynucleotide, nucleic acid, sugar cane centromere, sugar cane mini-chromosome, or vector of the invention.

In representative embodiments, the cell is a sugar cane plant cell (e.g., a sugar cane plant cell in vitro or a sugar cane plant cell in vivo in a plant part or plant).

Optionally, the plant cells, plant parts, seed and plants of the invention can comprise one or one or more (e.g., two, three, four, five, six, seven) mini-chromosomes of the invention. In the case of multiple mini-chromosomes, they can be the same and/or different in a single cell and/or throughout the plant, plant part, plant cells or seed of the invention.

In particular embodiments, the invention provides a sugar cane plant cell comprising a sugar cane mini-chromosome, wherein the sugar cane mini-chromosome is not integrated into the genome of the sugar cane plant cell. The sugar cane plant cell can optionally comprise a sugar cane mini-chromosome that comprises an exogenous nucleic acid, wherein the sugar cane plant cell optionally exhibits an altered phenotype associated with the expression of the exogenous nucleic acid. Alternatively, the exogenous nucleic acid can encode a functional, untranslated RNA that optionally produces a desired phenotype in the plant. The altered phenotype can be any phenotypic change of interest that can be detected and, optionally, measured. In an exemplary embodiment, the altered phenotype comprises altered expression (e.g., increased or decreased expression) of a native gene. In other embodiments, the altered phenotype comprises altered expression of an exogenous gene.

In embodiments of the invention, the mini-chromosome is not integrated into the plant genome. In other embodiments, the mini-chromosome is integrated into at least some plant genomes. In still further embodiments, some copies of the mini-chromosome can be integrated into the plant genome and others can be autonomous, either in the same cell and/or throughout the plant.

As yet a further aspect, the invention provides a sugar cane plant tissue, a sugar cane plant, and/or a sugar cane plant part comprising a sugar cane plant cell of the invention.

In another embodiment, the invention provides for sugar cane plants comprising any of the sugar cane mini-chromosomes of the invention, which may be referred to herein as “adchromosomal” sugar cane plants. In addition, the invention provides for sugar cane plant cells, tissues and seeds obtained from these modified plants.

In one embodiment, the invention provides for a sugar cane plant cell comprising any of the sugar cane mini-chromosomes of the invention that (i) is not integrated into the sugar cane plant cell genome and (ii) comprises an exogenous nucleic acid(s) that confers an altered phenotype on the sugar cane plant cell associated with the expression of at least one protein or functional, untranslated RNA within the sugar cane mini-chromosome. In representative embodiments, the altered phenotype can comprise increased expression of a native gene, decreased expression of a native gene, increased expression of an exogenous nucleic acid and/or decreased expression of an exogenous nucleic acid. In a further embodiment, these sugar cane plant cells also comprise one or more integrated exogenous nucleic acid(s).

Another embodiment of the invention is a part of any of the sugar cane plants of the invention. Exemplary sugar cane plant parts of the invention include a pod, root, sett root, shoot root, root primordial, shoot, primary shoot, secondary shoot, tassle, panicle, arrow, midrib, blade, ligule, auricle, dewlap, blade joint, sheath, node, internode, bud furrow, leaf scar, cutting, tuber, stem, stalk, fruit, berry, nut, flower, leaf, bark, wood, epidermis, vascular tissue, organ, protoplast, crown, callus culture, petiole, petal, sepal, stamen, stigma, style, bud, meristem, cambium, cortex, pith, sheath, silk, ovule or embryo. Other exemplary sugar cane plant parts are a meiocyte, gamete, ovule, pollen or endosperm of any of the plants described herein. Other exemplary plant parts are a seed, seed-piece, embryo, protoplast, cell culture, any group of plant cells organized into a structural and functional unit, ratoon, or propagule of any of the sugar cane plants of the invention.

An embodiment of the invention is a progeny of any of the sugar cane plants of the invention. These progeny of the invention may be the result of, for example, self-breeding, cross-breeding, apomyxis or clonal propagation. In exemplary embodiments, the invention also provides for progeny that comprise a sugar cane mini-chromosome that is descended from a parental sugar cane mini-chromosome that contained a centromere less than about 1000 kilobases in length, less than about 750 kilobases in length, less than about 600 kilobases in length, less than about 500 kilobases in length, less than about 400 kilobases in length, less than about 300 kilobases in length, less than about 250 kilobases in length, less than about 200 kilobases in length, less than about 150 kilobases in length, less than about 100 kilobases in length, less than about 90 kilobases in length, less than about 85 kilobases in length, less than about 80 kilobases in length, less than about 75 kilobases in length, less than about 70 kilobases in length, less than about 65 kilobases in length, less than about 60 kilobases in length, less than about 55 kilobases in length, less than about 50 kilobases in length, less than about 45 kilobases in length, less than about 40 kilobases in length, less than about 35 kilobases in length, less than about 30 kb in length, less than about 25 kilobases in length, less than about 20 kb in length, less than about 15 kilobases in length, less than about 12 kilobases in length, less than about 10 kb in length, less than about 7 kb in length, less than about 5 kb in length, or less than about 2 kb in length.

The invention also contemplates a sugar cane plant progeny comprising a sugar cane mini-chromosome of the invention, wherein the plant progeny is the result of breeding a sugar cane plant of the invention that comprises the sugarcane mini-chromosome.

As still another aspect, the invention provides a method of using a sugar cane plant of the invention, wherein the sugar cane plant comprises a sugar cane mini-chromosome of the invention comprising an exogenous nucleic acid encoding a protein, the method comprising growing the plant to produce the protein. Alternatively, the exogenous nucleic acid can encode a functional, untranslated RNA that optionally produces an altered phenotype in the plant. The method can optionally further comprise the step of harvesting and/or processing the sugar cane plant.

As still a further aspect, the invention provides a polynucleotide comprising, consisting essentially of, or consisting of a BAC nucleotide sequence, wherein the BAC nucleotide sequence is:

(a) the nucleotide sequence of BAC 18E23;

(b) the nucleotide sequence of BAC 17H17;

(c) the nucleotide sequence of BAC 21A4

(d) the nucleotide sequence of BAC 24J1;

(e) the nucleotide sequence of BAC 18P24;

(f) the nucleotide sequence of BAC 24F 15;

(g) the nucleotide sequence of BAC 17C9;

(h) the nucleotide sequence BAC 17P2;

(i) the nucleotide sequence of BAC 6C15;

(j) the nucleotide sequence of BAC 18J2;

(k) the nucleotide sequence of BAC 4H14; or

(l) the nucleotide sequence of BAC 17M9.

Also provided is a vector comprising a polynucleotide comprising a BAC nucleotide sequence of the invention and cells comprising a polynucleotide comprising, consisting essentially of, or consisting of a BAC nucleotide sequence or a vector comprising the same.

In a further embodiment, the invention provides for a sugar cane plant cell comprising a sugar cane mini-chromosome comprising a sugar cane centromere, wherein the centromere comprises (a) at least two repeat nucleotide sequences that have a sequence of any of SEQ ID NOS: 1-72 and/or a sequence that hybridizes under stringent conditions (e.g., comprising hybridization at 65° C. and washing three times for 15 minutes with 0.25×SSC, 0.1% SDS at 65° C.) to a nucleotide sequence of any of SEQ ID NOS: 1-72, and (b) at least one or at least two copies of a sugar cane CRS retroelement sequence (e.g., SEQ ID NO:74), and optionally wherein the centromere confers the ability to segregate to daughter sugar cane cells.

In another embodiment, the invention provides for a sugar cane plant cell comprising a sugar cane mini-chromosome comprising a sugar cane centromere, wherein the centromere comprises (a) at least two repeat nucleotide sequences that have a sequence of any of SEQ ID NOS: 1-72 and/or a sequence that is substantially identical to the nucleotide sequence of any of SEQ ID NOS: 1-72, and (b) at least one or at least two copies of a sugar cane CRS retroelement sequence (e.g., SEQ ID NO:74), and optionally wherein the centromere confers the ability to segregate to daughter sugar cane cells.

In representative embodiments, the invention also provides for sugar cane plant cells comprising a recombinant chromosome that has not been maintained in a cell of a heterologous organism.

In another embodiment, the invention provides for a sugar cane plant cell comprising (a) at least two copies of a repeated nucleotide sequence of any of SEQ ID NOS: 1-72 and/or that is substantially identical to a nucleotide sequence of any of SEQ ID NOS: 1-72 and/or hybridizes to the nucleotide sequence of any of SEQ ID NOS: 1-72 under stringent conditions (e.g., comprising hybridization at 65° C. and washing three times for 15 minutes with 0.25×SSC, 0.1% SDS at 65″C), and (b) an expression cassette comprising at least three exogenous nucleic acids, wherein the at least two copies of the repeated nucleotide sequence and the expression cassette are not integrated into the genome of the sugar cane plant cell.

The invention provides for any of the sugar cane mini-chromosomes described herein having a centromere comprising an array of repeated nucleotide sequence, wherein the array ranges from about 1 kb to about 200 kb in length, about 1 kb to about 150 kb in length, about 1 kb to about 125 kb in length, about 1 kb to about 100 kb in length, about 1 kb to about 75 kb in length, about 1 kb to about 50 kb in length, about 1 kb to about 25 kb in length, about 5 kb to about 200 kb in length, about 5 kb to about 150 kb in length, about 5 kb to about 125 kb in length, about 5 kb to about 100 kb in length, about 5 kb to about 75 kb in length, about 5 kb to about 50 kb in length, about 5 kb to about 25 kb in length, about 10 kb to about 200 kb in length, about 10 kb to about 150 kb in length, about 10 kb to about 100 kb in length, about 10 kb to about 75 kb in length, about 10 kb to about 50 kb in length, about 10 kb to about 25 kb in length, about 20 kb to about 200 kb in length, about 20 kb to about 150 kb in length, about 20 kb to about 100 kb in length, about 20 kb to about 75 kb in length, or about 20 kb to about 50 kb in length.

In other embodiments, the sugar cane mini-chromosomes of the invention comprise a centromere comprising an array of repeated nucleotide sequence, wherein the array is from about 21 kb to about 137 kb in length. To illustrate, the array can be about 44 kb, 69 kb, 83 kb, 85 kb, 96 kb, 103 kb, 108 kb, 132 kb or 137 kb. Alternatively, the array can be from about 6 kb to about 85 kb in length, for example, about 6 kb, 14 kb, 33 kb, 37 kb, 46 kb, 47 kb, 55 kb, 64 kb, 66 kb, 73 kb or 85 kb in length. In other particular embodiments, the array can be about 6 kb to 101 kb in length, for example, 6 kb, 21 kb, 38 kb, 45 kb, 47 kb, 51 kb, 56 kb, 68 kb, 70 kb, 74 kb or 101 kb in length.

The invention further contemplates any of the sugar cane mini-chromosomes of the invention having centromeres comprising at least about 300 bp, 400 bp, 500 bp, 600 bp, 700 bp, 750 bp, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, 10 kb, 10.5 kb, 11 kb, 11.5 kb, 12 kb, 12.5 kb, 13 kb, 13.5 kb, 14 kb, 14.5 kb, 15 kb, 16 kb, 17 kb, 18 kb, 19 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, 150 kb, 160 kb, 170 kb, 180 kb, 190 kb, 200 kb, 225 kb, 250 kb, 275 kb, 300 kb, 325 kb, 350 kb or 375 kb of nucleotide sequence and/or less than or equal to about 1000 kb, 900 kb, 800 kb, 700 kb, 600 kb, 500 kb, 400 kb, 300 kb, 200 kb, 190 kb, 150 kb, 100 kb, 95 kb, 90 kb, 85 kb, 80 kb, 75 kb, 70 kb, 65 kb, 60 kb, 55 kb, 50 kb, 45 kb, 40 kb, 35 kb, 30 kb, 28 kb, 25 kb, 20 kb, 17 kb, 15 kb, 12 kb, 10 kb, 7, kb, 6.4 kb, 5 kb, or 2 kb of nucleotide sequence (including all combinations as long as the lower limit is less than the upper limit).

In another embodiment, any of the sugar cane mini-chromosomes of the invention comprise centromeres having n copies of a repeated nucleotide sequence, wherein n is less than or equal to about 2000, 1500, 1000, 500, 400, 300, 250, 200, 100, 90, 80, 70, 60, 50, 40, 30, 25, 20, 15, 10, 9, 8, 7, 6 or 5. In exemplary embodiments, the centromeres of the sugar cane mini-chromosomes of the invention comprise n copies of a repeated nucleotide sequence, wherein n is at least about 2, 3, 4, 5, 6, 7, 8, 9, 10, 15, 20, 25, 30, 35, 40, 50, 60, 70, 80, 90, 100, 110, 120, 130, 140, 150, 160, 170, 180, 190, 200, 225, 250, 275, 300, 325, 350, 375, 400, 425, 450, 475, 500, 550, 600, 650, 700, 750, 800, 850, 900 or 1000. In additional exemplary embodiments, the centromeres of the sugar cane mini-chromosomes of the invention comprise n copies of a repeated nucleotide sequence where n ranges from about 2 to 10, about 2 to 20, about 2 to 50, about 2 to 100, about 2 to 150, about 2 to 200, about 2 to 250, about 2 to 300, about 2 to 400, about 2 to 500, about 2 to 600, about 2 to 700, about 2 to 800, about 2 to 900, about 2 to 1000, about 5 to 15, about 5 to 25, about 5 to 50, about 5 to 100, about 5 to 150, about 5 to 200, about 5 to 250, about 5 to 300, about 5 to 400, about 5 to 500, about 5 to 600, about 5 to 700, about 5 to 800, about 5 to 900, about 5 to 1000, about 15 to 25, about 15 to 50, about 15 to 100, about 15 to 150, about 15 to 200, about 15 to 250, about 15 to 300, about 15 to 400, about 15 to 500, about 15 to 600, about 15 to 700, about 15 to 800, about 15 to 900, about 15 to 1000, about 25 to 50, about 25 to 100, about 25 to 150, about 25 to 200, about 25 to 250, about 25 to 300, about 25 to 400, about 25 to 500, about 25 to 600, about 25 to 700, about 25 to 800, about 25 to 900, about 25 to 1000, about 40 to 100, about 40 to 150, about 40 to 250, about 40 to 300, about 40 to 400, about 40 to 500, about 40 to 600, about 40 to 700, about 40 to 800, about 40 to 900, or about 40 to 1000.

In exemplary embodiments, the sugar cane centromere or mini-chromosome comprises n copies of sugar cane satellite repeat sequence, where n ranges from about 42 to about 540. In other particular embodiments, n is about 483 (e.g., satellite repeat sequences based on SEQ ID NO:1), about 242 (e.g., satellite repeat sequences based on SEQ ID NO:2), about 621 (e.g., satellite repeat sequences based on SEQ ID NO:3), about 102 (e.g., satellite repeat sequences based on SEQ ID NO:4), about 346 (e.g., satellite repeat sequences based on SEQ ID NO:5), about 540 (e.g., satellite repeat sequences based on SEQ ID NO:6), about 339 (e.g., satellite repeat sequences based on SEQ ID NO:7), about 42 (e.g., satellite repeat sequences based on SEQ ID NO:8), about 276 (e.g., satellite repeat sequences based on SEQ ID NO:9), about 469 (e.g., satellite repeat sequences based on SEQ ID NO:10), about 408 (e.g., satellite repeat sequences based on SEQ ID NO:11), about 99 (e.g., satellite repeat sequences based on SEQ ID NO:12).

According to aspects of the invention, the size of the sugar cane satellite repeat sequence is at least about 30, 40, 50, 60, 70, 80, 90 or 100 bp in length and/or less than or equal to about 500, 400, 300, 250, 200, 175, 150 or 125 bp in length (in any combination as long as the lower limit is less than the upper limit). In representative embodiments, the sugar cane satellite centromere repeat sequence is from about 130 to 136 bp (e.g., satellite repeat sequences based on SEQ ID NO:1), from about 105 to 139 bp (e.g., satellite repeat sequences based on SEQ ID NO:2), from about 106 to 138 bp (e.g., satellite repeat sequences based on SEQ ID NO:3), from about 102 to 145 bp (e.g., satellite repeat sequences based on SEQ ID NO:4), from about 102 to 139 bp (e.g., satellite repeat sequences based on SEQ ID NO:5), from about 103 to 147 bp (e.g., satellite repeat sequences based on SEQ ID NO:6), from about 103 to 138 bp (e.g., satellite repeat sequences based on SEQ ID NO:7), from about 103 to 136 bp (e.g., satellite repeat sequences based on SEQ ID NO:8), from about 105 to 149 bp (e.g, satellite repeat sequences based on SEQ ID NO:9), from about 102 to 141 bp (e.g., satellite repeat sequences based on SEQ ID NO:10), from about 103 to 143 bp (e.g., satellite repeat sequences based on SEQ ID NO:11), from about 100 to 138 bp (e.g., satellite repeat sequences based on SEQ ID NO:12) about 136 bp (e.g., satellite repeat sequences based on SEQ ID NO:1), about 135 bp (e.g., satellite repeat sequences based on SEQ ID NO:2), about 137 bp (e.g., satellite repeat sequences based on SEQ ID NO:3), about 137 bp (e.g., satellite repeat sequences based on SEQ ID NO:4), about 137 bp (e.g., satellite repeat sequences based on SEQ ID NO:5), about 135 bp (e.g., satellite repeat sequences based on SEQ ID NO:6), about 136 bp (e.g., satellite repeat sequences based on SEQ ID NO:7), about 135 bp (e.g., satellite repeat sequences based on SEQ ID NO:8), about 133 bp (e.g, satellite repeat sequences based on SEQ ID NO:9), about 136 bp (e.g., satellite repeat sequences based on SEQ ID NO:10), about 136 bp (e.g., satellite repeat sequences based on SEQ ID NO:11), or about 133 bp (e.g., satellite repeat sequences based on SEQ ID NO:12). In other exemplary embodiments, the sugar cane satellite repeat sequence is from about 95, 96, 97, 98, 99 100, 101, 102, 103, 104 or 105 bp to about 125, 126, 127, 128, 129, 130, 131, 132, 133, 134, 135, 136, 137, 138, 139, 140, 141, 142, 143, 144, 145, 146, 147, 148, 149, 150, 151, 152, 153, 154 or 155 bp in length, in any combination, as long as the lower limit is less than the upper limit.

Exemplary sugar cane mini-chromosomes of the invention of the invention are contemplated to be at least about 300 bp, 400 bp, 500 bp, 600 bp, 700 bp, 750 bp, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb, 10 kb, 10.5 kb, 11 kb, 11.5 kb, 12 kb, 12.5 kb, 13 kb, 13.5 kb, 14 kb, 14.5 kb, 15 kb, 16 kb, 17 kb, 18 kb, 19 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, 150 kb, 160 kb, 170 kb, 180 kb, 190 kb, 200 kb, 225 kb, 250 kb, 275 kb, 300 kb, 325 kb, 350 kb or 375 kb in length and/or less than or equal to about 2000 kb, 1500 kb, 1000 kb, 900 kb, 800 kb, 700 kb, 600 kb, 500 kb, 450 kb, 400 kb, 350 kb, 300 kb, 250 kb, 200 kb, 150 kb, 100 kb, 80 kb, 60 kb, 40 kb, 35 kb, 25 kb, 10 kb, 5 kb, or 1 kb or less in length (in any combination as long as the lower limit is less than the upper limit)

For example, the sugar cane mini-chromosomes of the invention can be about 10 to 250 kb in length, about 10 to 150 kb in length, about 10 to 125 kb in length, about 10 to 100 kb in length, about 10 to 75 kb in length. In an exemplary embodiment, the sugar cane mini-chromosome is about 21 kb in length, about 44 kb in length, about 69 kb in length, about 83 kb in length, about 85 kb in length, about 96 kb in length, about 103 kb in length, about 108 kb in length, about 132 kb in length, or about 137 kb in length or ranges from about 21 to 137 kb in length.

In particular embodiments of the invention, the sugar cane mini-chromosome is from about 1 kb to about 2000 kb in size without vector sequence. For example, at least about 1, kb, 5 kb, 10 kb, 20 kb or 25 kb in size and/or less than or equal to about 2000 kb, 1500 kb, 1000 kb, 750 kb, 500 kb, 400 kb, 300 kb, 200 kb, 175 kb, 150 kb, 125 kb, 100 kb, 90 kb, 80 kb, 70 kb, 60 kb, 50 kb, 40 kb or 30 kb in size (with any combination being suitable as long as the lower limit is less than the upper limit) without vector sequences. To illustrate, the sugar cane mini-chromosome can be about 21 kb, 44 kb, 69 kb, 83 kb, 85 kb, 96 kb, 103 kb, 108 kb, 132 kb or 137 kb or range from about 21 kb to 137 kb without vector sequences.

Further, in some embodiments of the invention, the sugar cane centromere or mini-chromosome comprises from about 1 kb to about 150 kb of sugar cane satellite repeat content. For example, the satellite repeat content of the sugar cane centromere or mini-chromosome can be at least about 1 kb, 2 kb, 3 kb, 4 kb, 5 kb, 10 kb, 20 kb, or 30 kb and/or less than or equal to about 150 kb, 140 kb, 130 kb, 120 kb, 110 kb, 100 kb, 90 kb, 80 kb, 70 kb, 60 kb, 50 kb, 40 kb, 30 kb or 20 kb (with any combination being suitable as long as the lower limit is less than the upper limit).

To illustrate, the sugar cane satellite repeat content of the centromere or mini-chromosome can be about 6 kb, 14 kb, 33 kb, 37 kb, 46 kb, 47 kb, 55 kb, 64 kb, 66 kb, 73 kb or 85 kb or range from about 6 kb to 85 kb.

In representative embodiments, the sugar cane centromere or mini-chromosome comprises from about 0 to about 5, 10, 15, 20, 30, 40 or 50 kb of CRS retroelement sequence (e.g., SEQ ID NO:74). In embodiments of the invention, the term “CRS retroelement sequence” and similar terms indicate the nucleotide sequence of SEQ ID NO:74 and/or a nucleotide sequence that is substantially similar to SEQ ID NO:74 and/or a nucleotide sequence that hybridizes to SEQ ID NO:74 under stringent hybridization conditions, and fragments (e.g., at least about 50, 75, 100, 125, 150, 200, 300, 400, 500, 750, 1000, 1250, 1500, 1750, 2000, 2500, or 3000 bp) of any of the foregoing. Those skilled in the art will appreciate that fragments, rather than intact copies, of retroelement sequences are generally found around plant centromeres. For example, the sugar cane centromere or mini-chromosome can comprise at least about 500 bp, 600 bp, 700 bp, 800 bp, 900 bp, 1 kb, 1.5 kb, 2 kb, 2.5 kb, 3 bp, 3.5 kb, or 4 kb and/or less than or equal to about 50 kb, 40 kb, 30 kb, 25 kb, 20 kb, 17 kb, 15 kb, 12 kb, 10 kb, 9 kb, 8 kb or 5 kb of CRS retroelement sequence (with any combination being suitable as long as the lower limit is less than the upper limit) or a nucleotide sequence substantially identical thereto. In other embodiments, the sugar cane centromere or mini-chromosome comprises no or essentially no CRS retroelement sequence. In particular embodiments, the sugar cane centromere or mini-chromosome comprises about 800 bp, 900 bp, 4 kb, 5 kb, 6 kb, 7 kb, 8 kb or 16 kb or a range from about 800 bp to 16 kb of CRS retroelement sequence.

Accordingly, in embodiments of the invention, the sugar cane centromere specific repeat content (sugar cane satellite repeat sequences and CRS retroelement sequence) of the sugar cane centromere or mini-chromosome is at least about 1 kb, 2 kb, 3 kb, 4 kb, 5 kb, 10 kb, 15 kb, 20 kb, or 25 kb and/or less than or equal to about 200 kb, 175 kb, 150 kb, 125 kb, 110 kb, 100 kb, 90 kb, 80 kb, 70 kb, 60 kb, 50 kb, 40 kb, 30 kb or 20 kb (with any combination being suitable as long as the lower limit is less than the upper limit).

In representative embodiments, the centromere specific repeat content of the sugar cane centromere or mini-chromosome is about 6 kb, 21 kb, 38 kb, 45 kb, 47 kb, 52 kb, 56 kb, 68 kb, 70 kb, 74 kb or 101 kb, or ranges from about 6 to 101 kb.

The sugar cane centromere or mini-chromosome can optionally comprise other repeat sequences as well. In embodiments of the invention, the sugar cane centromere or mini-chromosome comprises at least about 0 to about 5, 10, 15, 20, 25, 30, 40 or 50 kb of other repeat sequences (i.e., other than sugar cane satellite sequence and/or CRS retroelement sequence). For example, the sugar cane centromere or mini-chromosome can comprise at least about 100 bp, 200 bp, 300 bp, 400 bp, 500 bp, 600 bp, 700 bp, 800 bp, 900 bp, 1 kb, 1.5 kb, 2 kb, 2.5 kb, 3 kb, 3.5 kb, or 4 kb and/or less than or equal to about 50 kb, 40 kb, 30 kb, 25 kb, 20 kb, 17 kb, 15 kb, 12 kb, 10 kb, 9 kb, 8 kb or 5 kb of other repeat sequences (with any combination being suitable as long as the lower limit is less than the upper limit). In other embodiments, the sugar cane centromere or mini-chromosome comprises no or essentially no other repeat sequences other than sugar cane satellite repeat sequence or CRS retroelement sequence.

In exemplary embodiments, the sugar cane centromere or mini-chromosome comprises about 800 bp, 2 kb, 3 kb, 5 kb, 7 kb, 9 kb, 16 kb or 21 kb or a range from about 800 bp to about 21 kb of other repeat sequences.

The sugar cane centromere or mini-chromosome can optionally comprise non-repeat content. In embodiments of the invention, the sugar cane centromere or mini-chromosome comprises at least about 0 to about 5, 10, 15, 20, 25, 30, 40, 50, 60, 70, 80, 90 or 100 kb of non-repeat sequence. For example, the sugar cane centromere or mini-chromosome can comprise at least about 100 bp, 200 bp, 300 bp, 400 bp, 500 bp, 1 kb, 1.5 kb, 2 kb, 2.5 kb, 3 kb, 3.5 kb, 4 kb, 5 kb, 5.5 kb, 6 kb, 7 kb, 7.5 kb or 8 kb and/or less than or equal to about 100 kb, 90 kb, 80 kb, 70 kb, 60 kb, 50 kb, 40 kb, 30 kb, 25 kb, 20 kb, 17 kb, 15 kb, 12 kb, 10 kb, 9 kb, 8 kb, 5 kb, 4 kb or 3 kb of non-repeat sequences (with any combination being suitable as long as the lower limit is less than the upper limit). In other embodiments, the centromere or mini-chromosome comprises no or essentially no non-repeat sequences.

In exemplary embodiments, the sugar cane centromere or mini-chromosome comprises about 8 kb, 11 kb, 14 kb, 17 kb, 27 kb, 29 kb, 33 kb, 34 kb, 36 kb, 42 kb or 55 kb or a range from about 8 kb to about 55 kb of non-repeat sequences.

In representative embodiments, the sugar cane centromeres or mini-chromosomes of the invention comprise any one or more of the features shown in Tables 5 to 21 of the working Examples, in any combination, as if such features and combinations were explicitly set forth in detail herein.

In an embodiment of the invention, any of the sugar cane mini-chromosomes of the invention comprise a centromere having at least 5 consecutive repeated nucleotide sequences of the invention in “head to tail orientation.” In an embodiment of the invention, any of the sugar cane mini-chromosomes of the invention comprise a centromere having at least 5 consecutive repeated nucleotide sequences in “tandem,” in which one repeat sequence is immediately adjacent to another repeat sequence in any orientation, e.g. head to tail, tail to tail, or head to head. The invention also provides for any of the sugar cane mini-chromosomes of the invention comprising a centromere having at least 5 repeated nucleotide sequences that are consecutive. The term “consecutive” refers to the same or substantially identical repeated nucleotide sequences (e.g., at least 70% identical) that follow one after another without being interrupted by other significant sequence elements. Consecutive repeated nucleotide sequences may be in any orientation, e.g. head to tail, tail to tail, or head to head, and need not be directly adjacent to each other (e.g., may be 1-50 bp apart).

The invention further provides for any of the sugar cane mini-chromosomes of the invention comprising a centromere having at least 5 of the consecutive repeated nucleotide sequences of the invention separated by less than n number of nucleotides, wherein n ranges from 1 to 10, or 1 to 20, or 1 to 30, or 1 to 40, or 1 to 50 or wherein n is less than 10 bp or n is less than 20 bp or n is less than 30 bp or n is less that 40 bp or n is less than 50 bp.

The invention also provides for any of the sugar cane mini-chromosomes of the invention comprising a centromere having at least two arrays of consecutive repeated nucleotide sequences of the invention, wherein the array comprises at least 2, 3, 4, 5, 6, 7, 8, 9, 10, 15, 20, 25, 30, 40, 50, 60, 70, 80, 90, 100, 150, 200, 250, 300, 350, 400, 450, 500, 600, 700, 800, 900, 1000 or 2000 repeated nucleotide sequences. The repeats within an array may be in tandem in any orientation, e.g. head to tail, tail to tail, or head to head, or consecutive in any orientation, e.g. head to tail, tail to tail, or head to head. The arrays may be separated by less than n number of nucleotides, wherein n ranges from 1 to 10, or 1 to 20, or 1 to 30, or 1 to 40, or 1 to 50, or 1 to 60, or 1 to 70, or 1 to 80, or 1 to 90, or 1 to 100, or wherein n is less than or equal to 10 bp, 20 bp, 30 bp, 40 bp or 50 bp. The at least two arrays may comprise the same repeated nucleotide sequence or different repeated nucleotide sequences (i.e. the first array can be comprised of repeat type 1 and a second array can be comprised of repeat type 2—here “type 1” and “type 2” are arbitrary designations).

The sugar cane mini-chromosomes of the invention optionally have a segregation efficiency during mitotic division of at least about 60%, at least about 80%, at least about 90% or at least about 95% and/or a transmission efficiency during meiotic division of, e.g., at least about 60%, at least about 80%, at least about 85%, at least about 90% or at least about 95%.

In another embodiment, the sugar cane mini-chromosomes of the invention comprise a site for site-specific recombination.

The invention also provides for a sugar cane mini-chromosome, wherein the mini-chromosome comprises at least one exogenous nucleic acid. In further exemplary embodiments, the sugar cane mini-chromosome comprises at least two or more, at least three or more, at least four or more, at least five or more, at least ten or more, at least 20 or more, at least 30 or more, at least 40 or more, or at least 50 or more exogenous nucleic acids. As described herein, the exogenous nucleic acid(s) can optionally encode a protein and/or a functional untranslated RNA (for example, that produces an altered phenotype in a plant).

In one embodiment, at least one exogenous nucleic acid of any of the sugar cane mini-chromosomes of the invention is operably linked to a regulatory sequence (e.g., a heterologous regulatory sequence) functional in plant cells, including but not limited to a plant regulatory sequence. The invention also provides for exogenous nucleic acids linked to a non-plant regulatory sequence, such as an arthropod, viral, bacterial, vertebrate or yeast regulatory sequence. The invention also provides for exogenous nucleic acids linked to a regulatory sequence from sugar cane.

The invention also provides for a mini-chromosome comprising a gene or group of genes that act to improve the total recoverable sugar from sugar cane. Such genes may act to increase the sugar concentration of the stem juice, increase the amount of juice, increase the stem strength to improve yield and/or increase total biomass of the plant. Such genes may be derived from bacterial sequences such as a sucrose isomerase or from animal, plant, fungal, or protist sequences. Such genes from plants may include genes involved in sugar metabolism or transport or genes of unknown function or genes not known to be associated with sugar metabolism and/or transport but that have been shown to quantitatively increase total recoverable sugar. Such genes may also include genes that affect plant height, stem diameter, water metabolism and/or total biomass. Such genes may also include those that regulate the equilibrium between starch and sugar. Several genes have been shown to improve sugar accumulation. For example, expression of a bacterial sucrose isomerase can increase sugar cane sugar content by as much as two-fold (Birch, R. G., and Wu, L. (2007). Doubled sugar content in sugar cane plants modified to produce a sucrose isomer. Plant Biotechnology Journal 5: 109-117). The lignin-deficient “brown midrib” mutations improve sorghum sugar content via their effects on lignin; this phenotype is caused by mutations in cinnamyl alcohol dehydrogenase (CAD), and 14 CAD-like genes are present in the sorghum genome (Saballos, A., Ejeta, G., Sanchez, E., Kang, C., and Vermerris, W. (2008). A Genome-Wide Analysis of the Cinnamyl Alcohol Dehydrogenase Family in Sorghum (Sorghum bicolor (L.) Moench) Identifies SbCAD2 as the Brown midrib6 Gene. Genetics).

In another embodiment, the sugar cane mini-chromosome comprises an exogenous nucleic acid that comprises a QTL that confers a desirable trait. QTLs that affect total recoverable sugars have been mapped in sugar cane (Murray, S. C., Sharma, A., Rooney, W. L., Klein, P., Mullet, J. E., Mitchell, S. E., Kresovitch, S. (2008) Genetic Improvement of Sorghum as a Biofuel Feedstock: I. QTL for Stem Sugar and Grain Nonstructural Carbohydrates. Crop Sci. 48:2165-2179).

In another embodiment, the sugar cane mini-chromosome comprises an exogenous nucleic acid that confers herbicide resistance, insect resistance, disease resistance, or stress resistance on the sugar cane plant. The invention provides for sugar cane mini-chromosomes comprising an exogenous nucleic acid that confers resistance to phosphinothricin or glyphosate herbicide. Nonlimiting examples include an exogenous nucleic acid that encodes a phosphinothricin acetyltransferase, glyphosate acetyltransferase, acetohydroxyadic synthase or a mutant enoylpyruvylshikimate phosphate (EPSP) synthase. Nonlimiting examples of exogenous nucleic acids that confer insect resistance include a Bacillus thuringiensis toxin gene or Bacillus cereus toxin gene. In related embodiments, the sugar cane mini-chromosome comprises an exogenous nucleic acid conferring herbicide resistance, an exogenous nucleic acid conferring insect resistance, and optionally at least one additional exogenous nucleic acid.

The invention further provides for sugar cane mini-chromosomes comprising additional copies of genes already found in the sugar cane genome. The invention also provides for the additional copies of sugar cane genes carried on the sugar cane mini-chromosome to be operably linked to either their native regulatory sequences or to heterologous regulatory sequences.

The invention further provides for sugar cane mini-chromosomes comprising an exogenous nucleic acid that confers resistance to drought, heat, chilling, freezing, excessive moisture, ultraviolet light, ionizing radiation, toxins, pollution, mechanical stress or salt stress. The invention also provides for a sugar cane mini-chromosome that comprises an exogenous nucleic acid that confers resistance to a virus, bacteria, fungi or nematode.

The invention provides for sugar cane mini-chromosomes comprising an exogenous nucleic acid(s) including but not limited to a nitrogen fixation gene, a plant stress-induced gene, a nutrient utilization gene, a gene that affects plant pigmentation, a gene that encodes an antisense or ribozyme molecule, a gene encoding a secretable antigen, a toxin gene, a receptor gene, a ligand gene, a seed storage gene, a hormone gene, an enzyme gene, an interleukin gene, a clotting factor gene, a cytokine gene, an antibody gene, a growth factor gene, a transcription factor gene, a transcriptional repressor gene, a DNA-binding protein gene, a recombination gene, a DNA replication gene, a programmed cell death gene, a kinase gene, a phosphatase gene, a G protein gene, a cyclin gene, a cell cycle control gene, a gene involved in transcription, a gene involved in translation, a gene involved in RNA processing, a gene involved in RNAi, an organellar gene, a intracellular trafficking gene, an integral membrane protein gene, a transporter gene, a membrane channel protein gene, a cell wall gene, a gene involved in protein processing, a gene involved in protein modification, a gene involved in protein degradation, a gene involved in metabolism, a gene involved in biosynthesis, a gene involved in assimilation of nitrogen or other elements or nutrients, a gene involved in controlling carbon flux, a gene involved in respiration, a gene involved in photosynthesis, a gene involved in light sensing, a gene involved in organogenesis, a gene involved in embryogenesis, a gene involved in differentiation, a gene involved in meiotic drive, a gene involved in self incompatibility, a gene involved in development, a gene involved in nutrient, metabolite or mineral transport, a gene involved in nutrient, metabolite or mineral storage, a calcium-binding protein gene and/or a lipid-binding protein gene.

The invention also provides for a sugar cane mini-chromosome comprising an exogenous enzyme gene including but not limited to a gene that encodes an enzyme involved in metabolizing biochemical wastes for use in bioremediation, a gene that encodes an enzyme for modifying pathways that produce secondary plant metabolites, a gene that encodes an enzyme that produces a pharmaceutical, a gene that encodes an enzyme that improves changes in the nutritional content of a plant, a gene that encodes an enzyme involved in vitamin synthesis, a gene that encodes an enzyme involved in carbohydrate, polysaccharide or starch synthesis, a gene that encodes an enzyme involved in mineral accumulation or availability, a gene that encodes a phytase, a gene that encodes an enzyme involved in fatty acid, fat or oil synthesis, a gene that encodes an enzyme involved in synthesis of chemicals or plastics, a gene that encodes an enzyme involved in synthesis of a fuel, a gene that encodes an enzyme involved in synthesis of a fragrance, a gene that encodes an enzyme involved in synthesis of a flavor, a gene that encodes an enzyme involved in synthesis of a pigment or dye, a gene that encodes an enzyme involved in synthesis of a hydrocarbon, a gene that encodes an enzyme involved in synthesis of a structural or fibrous compound, a gene that encodes an enzyme involved in synthesis of a food or animal feed additive, a gene that encodes an enzyme involved in synthesis of a chemical insecticide, a gene that encodes an enzyme involved in synthesis of an insect repellent and/or a gene controlling carbon flux in a plant.

In another embodiment of the invention, any of the sugar cane mini-chromosomes of the invention comprise one or more telomeres (e.g., one or two).

The invention also provides embodiments wherein any of the sugar cane mini-chromosomes of the invention are linear or circular. According to an illustrative embodiment, the mini-chromosome is linear with a telomere at one or both termini.

In another aspect, the invention provides for methods of making a sugar cane mini-chromosome for use in any of the sugar cane plants of the invention. In representative embodiments, these methods comprise identifying a centromere nucleotide sequence in a sugar cane genomic DNA library using a multiplicity of diverse probes, and constructing a sugar cane mini-chromosome comprising the centromere nucleotide sequence. These methods may further comprise determining hybridization scores for hybridization of the multiplicity of diverse probes to genomic clones within the sugar cane genomic nucleic acid library, determining a classification for genomic clones within the sugar cane genomic nucleic acid library according to the hybridization scores for at least two of the diverse probes, and selecting one or more genomic clones within one or more classifications for constructing the sugar cane mini-chromosome.

The invention also contemplates methods of using any of the sugar cane plants of the invention to produce a recombinant protein, for example, by growing a sugar cane plant comprising a sugar cane mini-chromosome that comprises an exogenous nucleic acid encoding the desired recombinant protein. Optionally the sugar cane plant is harvested and the desired protein product is isolated from the plant. Exemplary protein products include industrial enzymes such as those useful for biofuel production.

The invention also contemplates methods of using any of the sugar cane plants of the invention to produce a chemical product, for example, by growing a sugar cane plant comprising a sugar cane mini-chromosome that comprises an exogenous nucleic acid encoding an enzyme involved in the synthesis of the chemical product. Optionally the sugar cane plant is harvested and the desired chemical product is isolated from the plant. Exemplary chemical products include sugars, lipids and carbohydrates useful in the production of biofuels.

Another aspect of the invention provides for methods of using any of the sugar cane plants, plant parts, plant tissues or plant cells of the invention comprising a sugar cane mini-chromosome for a food or feed product, a pharmaceutical product or chemical product, according to which a suitable exogenous nucleic acid is expressed in sugar cane plants, plant parts, plants tissues or plant cells and the plant, plant part, plant tissue or plant cells are grown. The plant, plant part, plant tissue or plant cells may secrete the product into its growth environment or the product may be contained within the plant, plant part, plant tissue or plant cells, in which case the plant, plant part, plant tissue or plant cells are harvested and, optionally, desired products are extracted.

Thus, the invention contemplates methods of using any of the sugar cane plants of the invention comprising a sugar cane mini-chromosome to produce a modified food or animal feed product, for example, by growing a plant that expresses an exogenous nucleic acid that alters the nutritional content of the plant, and harvesting and/or processing the sugar cane plant.

In another embodiment, the invention provides for methods of contacting a sugar cane cell with a sugar cane mini-chromosome comprising the steps of (a) delivering the mini-chromosome to immature differentiated leaves of the apical region of the stem of a sugar cane plant, wherein the mini-chromosome comprises a selectable marker gene, and (b) selecting the sugar cane cells expressing the marker gene, wherein expression of the marker gene indicates transformation with the mini-chromosome. The leaves used in this method are immature but are fully differentiated, such as the inner immature leaves of the sugar cane stem. In an exemplary embodiment, the mini-chromosome may be delivered by bombarding the immature leaves with micro-particles comprising the sugar cane mini-chromosome.

The invention also provides for methods of regenerating a sugar cane plant transformed with a sugar cane mini-chromosome comprising the steps of (a) obtaining a callus comprising a sugar cane cell that is transformed with a sugar cane mini-chromosome of the invention (e.g., by any of the methods of the invention), and (b) growing the callus in medium that optionally comprises 1%-3% polyvinylpyrrolidone to form a plantlet, wherein the cells of the plantlet are transformed with the sugar cane mini-chromosome. In a further embodiment, the methods of culturing the callus comprise growing the cells in liquid media for a time period and subsequently culturing the cells in a solid culture media. In an exemplary embodiment, the sugar cane mini-chromosome comprises a nucleic acid that encodes proteins that regulate growth such as a protein(s) in the auxin biosynthesis or perception pathways. Such proteins include without limitation Trp mono-oxygenase, Indole-3-acetamide hydrolase, and AMP iso-pentenyl transferase. When these three proteins are expressed on a mini-chromosome(s), Trp mono-oxygenase converts Trp into indole-3-acetamide, which indole-3 acetamide hydrolase converts into auxin. AMP iso-pentenyl transferase converts AMP into a cytokinin. The expression of all three proteins allows a cultured cell to grow in the absence of exogenously supplied hormones. Another embodiment is a method of stably incorporating an autonomously replicating nucleic acid into a sugar cane plant cell comprising the steps of transforming a sugar cane plant cell with a polynucleotide, nucleic acid, sugar cane centromere, or mini-chromosome of the invention; and obtaining a sugar cane plant cell wherein the nucleic acid is autonomously replicating during sugar cane plant cell division, and wherein the nucleic acid segregates to daughter sugar cane cells during cell division.

These and other aspects of the invention are described further in the detailed description of the invention that follows. Further, it is explicitly intended that the various aspects of the invention described herein may be used in any combination according to different embodiments of the invention.

### DETAILED DESCRIPTION OF THE INVENTION

While this invention is susceptible of embodiment in many different forms, and will be described herein in detail, specific embodiments thereof with the understanding that the present disclosure is to be considered as an exemplification of the principles of the invention and is not intended to limit the invention to the specific embodiments illustrated.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

### DEFINITIONS

As used in the description of the invention and the appended claims, the singular forms “a,” “an” and “the” are intended to include the plural forms as well, unless the context clearly indicates otherwise.

The term “about,” as used herein when referring to a measurable value such as the length of a polynucleotide or polypeptide sequence, time, temperature, and the like, is meant to encompass variations of ±20%, ±10%, ±5%, ±1%, ±0.5%, or even ±0.1% of the specified amount. In some instances, the term “about” may also refer to a value that has been rounded up or down to the nearest whole number.

As used herein, “and/or” refers to and encompasses any and all possible combinations of one or more of the associated listed items, as well as the lack of combinations when interpreted in the alternative (“or”).

The term “adchromosomal” sugar cane plant or plant part as used herein means a sugar cane plant or plant part that contains at least one sugar cane mini-chromosome, optionally a functional, stable and autonomous sugar cane mini-chromosome. Adchromosomal sugar cane plants or plant parts may be chimeric or not chimeric (chimeric meaning that sugar cane mini-chromosomes are only in certain portions of the plant, and are not uniformly distributed throughout the plant). An adchromosomal sugar cane plant cell contains at least one sugar cane mini-chromosome, optionally a functional, stable and autonomous sugar cane mini-chromosome. An “adchromosomal” sugar can plant, plant part or plant cell can comprise one or one or more (e.g., two, three, four, five or more) sugar cane mini-chromosomes, which can be the same and/or different from each other.

The term “autonomous” as used herein means that when delivered to plant cells, at least some sugar cane mini-chromosomes are transmitted through mitotic division to daughter cells and are episomal in the daughter plant cells, i.e. are not chromosomally integrated in the daughter plant cells. Daughter plant cells that contain autonomous mini-chromosomes can be selected for further replication using, for example, selectable or screenable markers. During the introduction into a cell of a mini-chromosome, or during subsequent stages of the cell cycle, there may be chromosomal integration of some portion or all of the DNA derived from a mini-chromosome in some cells. The mini-chromosome is still characterized as autonomous despite the occurrence of such events if a plant may be regenerated that contains episomal descendants of the mini-chromosome, optionally distributed throughout its parts, or if gametes or progeny can be derived from the plant that contain episomal descendants of the mini-chromosome distributed through its parts.

As used herein, a “centromere” is any DNA sequence that confers an ability to segregate to daughter cells through cell division. In one embodiment, this sequence may produce a transmission efficiency to daughter cells ranging from about 1% to about 100%, including from about 1%, 5%, 10%, 15%, 20%, 30% or 40% to about 5%, 10%, 20%, 30%, 40%, 50%, 60%, 70%, 80%, 90% or about 95% (including any combination as long as the lower limit is less than the upper limit) of daughter cells. In embodiments of the invention, the transmission efficiency to daughter cell is at least about 1%, at least about 5%, at least about 10%, at least about 15%, at least about 30%, at least about 40%, at least about 50%, at least about 60%, at least about 70% or more. Variations in transmission efficiency may find important applications within the scope of the invention; for example, mini-chromosomes carrying centromeres that confer 100% stability can be maintained in all daughter cells without selection, while those that confer 1% stability can be temporarily introduced into a transgenic organism, but be eliminated when desired. In particular embodiments of the invention, the centromere may confer stable transmission to daughter cells of a nucleic acid sequence, including a nucleic acid construct (e.g., a recombinant construct) comprising the centromere, through mitotic or meiotic divisions, including through both meiotic and meiotic divisions. A plant centromere is not necessarily derived from plants, but has the ability to promote DNA transmission to daughter plant cells.

In embodiments of the invention, a “sugar cane centromere” means a polynucleotide sequence having the properties of a centromere that is assembled or derived from one or more fragments of a native centromere(s) (e.g., native sugar cane centromere(s)) and/or other polynucleotide sequence, which are (i) isolated from a plant cell (e.g., a sugar cane plant cell), and/or based on plant centromere sequence motifs (e.g., sugar cane plant centromere sequence motifs), and optionally (ii) inserted into a vector (e.g. a plasmid vector) that is propagated and maintained in a cell of a heterologous organism, and (iii) delivered back into a sugar cane plant cell as part of a mini-chromosome of the invention. In embodiments of the invention, the centromere is from sugar cane genomic DNA. According to this embodiment, the sugar cane centromere may be modified by an endogenous in vivo process after it is delivered into a sugar cane plant cell such that its sequence now differs from that contained in the parental sugar cane mini-chromosome as propagated in a cell of a heterologous organism. For the avoidance of doubt, this embodiment does not encompass derivatives or deletions of native sugar cane centromeres that are constructed within the sugar cane plant cell, and are never maintained in their entirety in a cell of a heterologous organism.

As used herein, the term “circular permutations” refer to variants of a sequence that begin at base n within the sequence, proceed to the end of the sequence, resume with base number one of the sequence, and proceed to base n−1. For this analysis, n may be any number less than or equal to the length of the sequence. For example, circular permutations of the sequence ABCD are: ABCD, BCDA, CDAB, and DABC.

The term “coding sequence” is defined herein as a nucleic acid sequence that is transcribed into mRNA which is translated into a polypeptide when placed under the control of promoter sequences. The boundaries of the coding sequence are generally determined by the ATG start codon located at the start of the open reading frame, near the 5′ end of the mRNA, and TAG, TGA or TAA stop codons at the end of the coding sequence, near the 3′ end of the mRNA, and in some cases, a transcription terminator sequence located just downstream of the open reading frame at the 3′ end of the mRNA. A coding sequence can include, but is not limited to, genomic DNA, cDNA, semisynthetic, synthetic, or recombinant nucleic acid sequences.

As used herein the term “consensus” refers to a nucleic acid sequence derived by comparing two or more related sequences. A consensus sequence defines both the conserved and variable sites between the sequences being compared. Any one of the sequences used to derive the consensus or any permutation defined by the consensus may be useful in the construction of mini-chromosomes.

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein the term “consisting essentially of” (and grammatical variations) means that the composition, article, product or method does not comprise any elements that materially change the functioning of the composition, article, product or method other than those elements specifically recited.

The term “exogenous” when used in reference to a nucleic acid, for example, is intended to refer to any nucleic acid that has been introduced into a recipient cell, regardless of whether the same or similar nucleic acid is already present in such a cell. Thus, as an example, “exogenous DNA” can include an additional copy of DNA that is already present in the plant cell, DNA from another plant, DNA from a different organism, or a DNA generated externally, such as a DNA sequence containing an antisense message of a gene, or a DNA sequence encoding a synthetic or modified version of a gene. An “exogenous gene” can be a gene not normally found in the host genome in an identical context, or an extra copy of a host gene. The gene may be isolated from a different species than that of the host genome, or alternatively, isolated from the host genome but operably linked to one or more regulatory regions which differ from those found in the unaltered, native gene.

Expression cassettes are well-known in the art. For example, the term “expression cassette” as used herein describes a nucleotide sequence comprising a transcriptional initiation region that may be linked to a nucleic acid(s) or gene(s) of interest. The transcriptional initiation region, (e.g., a promoter) may be native or heterologous (e.g., foreign) to the host, and can be a natural or synthetic sequence. The expression cassette may optionally be provided with a plurality of restriction sites for insertion of the gene(s) or nucleic acid(s) of interest to be under the transcriptional regulation of the regulatory regions. In particular embodiments of the invention, the mini-chromosome comprises one expression cassette. In other embodiments, the mini-chromosome comprises two or more expression cassettes, each of which encodes at least one gene or nucleic acid of interest. The expression cassette may additionally contain 5′ leader sequences. Such leader sequences are known in the art and can act to enhance translation. Translation leaders are also well-known in the art. Optionally, the expression cassette comprises a selectable marker gene for the selection of transformed cells.

The term “functional” as used herein to describe a mini-chromosome means that when an exogenous nucleic acid is present within the mini-chromosome the exogenous nucleic acid can function in a detectable manner when the mini-chromosome is within a plant cell. Exemplary functions of the exogenous nucleic acid include transcription of the exogenous nucleic acid; expression of the exogenous nucleic acid; regulatory control of expression of other exogenous nucleic acids; recognition by a restriction enzyme or other endonuclease, ribozyme or recombinase; providing a substrate for DNA methylation, DNA glycolation or other DNA chemical modification; binding to proteins such as histones, helix-loop-helix proteins, zinc binding proteins, leucine zipper proteins, MADS box proteins, topoisomerases, helicases, transposases, TATA box binding proteins, viral proteins, reverse transcriptases, or cohesins; providing an integration site for homologous recombination; providing an integration site for a transposon, T-DNA or retrovirus; providing a substrate for RNAi synthesis; priming of DNA replication; aptamer binding and/or kinetochore binding. If multiple exogenous nucleic acids are present within the mini-chromosome, the function of one or more of the exogenous nucleic acids can be detected under suitable conditions permitting function thereof.

Unless the context indicates otherwise, the term “gene” is not intended to be limited to a nucleic acid as it exists in its native state in the genome of an organism or virus, e.g., including the native introns and regulatory sequences such as promoter, initiation and termination sequences. Thus, unless indicated otherwise by context, as used herein the term “gene” is construed more broadly as a nucleic acid encoding a protein or functional, untranslated RNA.

Exemplary “hybridization” conditions are provided herein. Guidance for performing hybridization reactions can be found in Current Protocols in Molecular Biology (1989) John Wiley & Sons, N.Y., 6.3.1-6.3.6. Aqueous and non-aqueous methods are described in that reference and either can be used. For example, “low stringency” hybridization conditions can comprise hybridization in 6× sodium chloride/sodium citrate (SSC) at about 45° C., followed by two washes in 0.5×SSC, 0.1% SDS, at least at 50° C. An illustration of “medium stringency” hybridization conditions are hybridization in 6×SSC at about 45° C., followed by one or more washes in 0.2×SSC, 0.1% SDS at 55° C. One example of “stringent” hybridization conditions comprise hybridization at 65° C. and washing three times for 15 minutes with 0.25×SSC, 0.1% SDS at 65° C. Additional exemplary stringent hybridization conditions comprise hybridization in 0.02 M to 0.15 M NaCl at temperatures of about 50° C. to 70° C. or 0.5×SSC 0.25% SDS at 65° for 15 minutes, followed by a wash at 65° C. for a half hour or hybridization at 65° C. for 14 hours followed by 3 washings with 0.5×SSC, 1% SDS at 65° C. Other exemplary highly selective or stringent hybridization conditions comprise 0.02 M to 0.15 M NaCl at temperatures of about 50° C. to 70° C. or 0.5×SSC 0.25% SDS at 65° for 12-15 hours, followed three washes at 65° C. for 15-90 minutes each. Probe hybridization can be scored visually to determine a binary (positive versus negative) value, or the probes can be assigned a score based on the relative strength of their hybridization on a 10 point scale. For example, relative hybridization scores of 5 may be used to select clones that hybridize well to the probe. Alternatively, a hybridization signal greater than background for one or more of these probes can be used to select clones.

The nucleic acids, polynucleotides and centromere sequences of the invention are optionally “isolated.” An “isolated” nucleic acid molecule, polynucleotide, centromere sequence (and the like) is a nucleic acid molecule, polynucleotide or centromere sequence that, by the hand of man, exists apart from its native environment and, is therefore not a product of nature. An isolated nucleic acid molecule, polynucleotide or centromere sequence may exist in a purified form or may exist in a non-native environment such as, for example, a recombinant host cell. Thus, for example, the term “isolated” means that it is separated from the chromosome and/or cell in which it naturally occurs. A nucleic acid, polynucleotide or centromere sequence is also isolated if it is separated from the chromosome and/or cell in which it naturally occurs and is then inserted into a genetic context, a chromosome, a chromosome location, a nucleic acid and/or a cell in which it does not naturally occur. The recombinant nucleic acids, polynucleotides and centromere sequences of the invention can be considered to be “isolated.”

An “isolated” nucleic acid, polynucleotide or centromere sequence (and similar terms) can refer to a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. The term “isolated” can refer to a polynucleotide, nucleic acid or centromere sequence that is substantially free of cellular material, viral material, and/or culture medium (when produced by recombinant DNA techniques), or chemical precursors or other chemicals (when chemically synthesized). “Isolated” does not necessarily mean that the preparation is technically pure (homogeneous), but it is sufficiently pure to provide the polynucleotide or nucleic acid in a form in which it can be used for the intended purpose. In certain embodiments, the isolated polynucleotide, nucleic acid or centromere sequence is at least about 50% pure, e.g., at least about 60%, 70%, 80%, 90%, 95%, 96%, 97%, 98%, or 99% or more pure.

An “isolated” cell refers to a cell that is at least partially separated from other components with which it is normally associated in its natural state. For example, an isolated cell can be a cell in culture medium.

As used herein, a “library” is a pool of cloned DNA fragments that represents some or all DNA sequences collected, prepared or purified from a specific source. Each library may contain the DNA of a given organism inserted as discrete restriction enzyme generated fragments or as randomly sheared fragments into many thousands of plasmid vectors. For purposes of the present invention, E. coli, yeast, and Salmonella plasmids are particularly useful for propagating the genome inserts from other organisms. In principle, any gene or sequence present in the starting DNA preparation can be isolated by screening the library with a specific hybridization probe (see, for example, Young et al., In: Eukaryotic Genetic Systems ICN-UCLA Symposia on Molecular and Cellular Biology, VII, 315-331, 1977).

As used herein, the term “linker” refers to a DNA molecule, generally up to 50 or 60 nucleotides long and composed of two or more complementary oligonucleotides that have been synthesized chemically, or excised or amplified from existing plasmids or vectors. In a representative embodiment, the linker comprises one, or more than one, restriction enzyme site for a blunt cutting enzyme and/or a staggered cutting enzyme, such as BamHI. In other representative embodiments, one end of the linker is designed to be ligatable to one end of a linear DNA molecule and the other end is designed to be ligatable to the other end of the linear molecule, or both ends may be designed to be ligatable to both ends of the linear DNA molecule.

As used herein, a “mini-chromosome” is a recombinant DNA construct including a centromere that is capable of transmission to daughter cells. A mini-chromosome may remain separate from the host genome (as episomes) or may integrate into host chromosomes. The stability of this construct through cell division can range between from about 1% to about 100%, including at least about 5%, 10%, 20%, 30%, 40%, 50%, 60%, 70%, 80%, 90% or about 95%. The mini-chromosome construct may be a circular or linear molecule. It may include elements such as one or more telomeres, origin of replication sequences, stuffer sequences, buffer sequences, chromatin packaging sequences, linkers and/or exogenous nucleic acids. The number of such sequences included is only limited by the physical size limitations of the construct itself. In representative embodiments, the mini-chromosome can contain DNA derived from a native centromere (e.g., from sugar cane genomic DNA). In representative embodiments, the amount of DNA from the native centromere is the minimal amount to obtain a transmission efficiency in the range of 1-100%. The mini-chromosome may optionally also contain a synthetic centromere composed of tandem arrays of repeats of any sequence, either derived from a native centromere, or of synthetic DNA. The mini-chromosome can further optionally comprise multiple restriction sites for insertion of one or more exogenous nucleic acids. Alternatively, the mini-chromosome may also contain DNA derived from multiple native centromeres. The mini-chromosome may be inherited through mitosis or meiosis, or through both meiosis and mitosis. As used herein, the term mini-chromosome also encompasses recombinant chromosomes. In embodiments of the invention, the mini-chromosome is not a recombinant chromosome.

In representative embodiments, the sugar cane minichromosome is a recombinant DNA construct that when present within a sugar cane cell is capable of mitotic and/or meiotic transmission to sugar cane daughter cells under appropriate conditions and comprises a sugar cane centromere and, optionally, one or more of the following:
(a) one or more telomeres;
(b) one or more sequences for regulating, maintaining and/or imparting topological and/or chromatin structure, molecular integrity and/or stability of gene expression and/or inheritance in sugar cane;
(c) vector DNA that allows for propagation of the mini-chromosome in sugar cane and DNA that facilitates the selective removal of unwanted portions of the mini-chromosome prior to or after sugar cane transformation; and/or
(d) an expression cassette, wherein (i) the expression cassette serves to regulate, maintain, or impart function or stability to a mini-chromosome in sugar cane; or (ii) the expression cassette imparts one or more functions other than to regulate, maintain, or impart function or stability to the mini-chromosome.

The term “non-protein expressing sequence,” “non-protein coding sequence,” “functional RNA” or “functional untranslated RNA” is defined herein as a nucleic acid sequence that is not eventually translated into protein, but nonetheless is functional. The nucleic acid may or may not be transcribed into RNA. Exemplary sequences include ribozymes, antisense RNA, or RNAi.

As used herein, “nucleic acid,” “nucleotide sequence,” and “polynucleotide” are used interchangeably and encompass both RNA and DNA, including cDNA, genomic DNA, mRNA, partially or wholly synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of the invention. Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR §1.822 and established usage.

The term “operably linked” is defined herein as a configuration in which a control sequence, e.g., a promoter sequence, directs transcription or translation of another sequence, for example a coding sequence. For example, a promoter sequence can be appropriately placed at a position relative to a coding sequence such that the control sequence directs the production of a polypeptide encoded by the coding sequence.

“Phenotype” or “phenotypic trait(s)”, as used herein, refers to an observable property or set of properties resulting from the expression of a nucleic acid. The set of properties may be observed visually or after biological or biochemical testing, and may be constantly present or may only manifest upon challenge with the appropriate stimulus or activation with the appropriate signal.

The term “recombinant chromosome” refers to an engineered mini-chromosome that has been constructed by fragmenting a native chromosome and identifying fragmentation products that are capable of segregation through mitotic and/or meiotic cell divisions. Recombinant chromosomes are generally not constructed in vitro from constituent parts and have not been passaged through an heterologous cell such as a bacteria or fungus (as is commonly used in standard cloning techniques). Recombinant chromosomes may be used as targets for addition of expression cassettes.

The term “sugar cane” refers to any species or hybrid of the genus Saccharum now known or later identified including but not limited to: S. acinaciforme, S. aegyptiacum, S. alopecuroides (Silver Plume Grass), S. alopecuroideum, S. alopecuroidum (Silver Plumegrass), S. alopecurus, S. angustifolium, S. antillarum, S. arenicola, S. argenteum, S. arundinaceum (Hardy Sugar Cane (Usa)), S. arundinaceum var. trichophyllum, S. asper, S. asperum, S. atrorubens, S. aureum, S. balansae, S. baldwini, S. baldwinii (Narrow Plumegrass), S. barberi (Cultivated sugar cane), S. barbicostatum, S. beccarii, S. bengalense (Munj Sweetcane), S. benghalense, S. bicorne, S. biflorum, S. boga, S. brachypogon, S. bracteatum, S. brasilianum, S. brevibarbe (Short-Beard Plume Grass), S. brevibarbe var. brevibarbe (Shortbeard Plumegrass), S. brevibarbe var. contortum (Shortbeard Plumegrass), S. brevifolium, S. brunneum, S. caducum, S. canaliculatum, S. capense, S. casi, S. caudatum, S. cayennense, S. cayennense var. genuinum, S. cayennense var. laxiusculum, S. chinense, S. ciliare, S. coarctatum (Compressed Plumegrass), S. confertum, S. conjugatum, S. contortuni, S. contortion var. contortion, S. contraction, S. cotuliferum, S. cylindricum, S. cylindricum var. contractum, S. cylindricum var. longifolium, S. deciduum, S. delimit, S. diandrum, S. dissitiflorum, S. distichophyllum, S. dubium, S. ecklonii, S. edule, S. elegans, S. elephantinum, S. erianthoides, S. europaeum, S. exaltatum, S. fasciculatum, S. fastigiatum, S. fatuum, S. filifolium, S. filiforme, S. floridulum, S. formosanum, S. fragile, S. fulvum, S. fuscum, S. giganteum (sugar cane Plume Grass), S. glabruni, S, glaga, S. glaucum, S. glaza, S. grandiflorum, S. griffithii, S. hildebrandtii, S. hirsutum, S. holcoides, S. holcoides var. warmingianum, S. hookeri, S. hybrid, S. hybridum, S. Indian, S. infirmum, S. insulare, S. irritans, S. jaculatorium, S. jamaicense, S. japonicum, S. juncifolium, S. kajkaiense, S. kanashiroi, S. klagha, S. koenigii, S. laguroides, S. longifolium, S. longisetosum, S. longisetosum var. hookeri, S. longisetum, S. Iota, S. luzonicum, S. macilentum, S. macrantherum, S. maximum, S. mexicanum, S. modhara, S. monandrum, S. moonja, S. munja, S. munroanum, S. muticum, S. narenga (Narenga sugar cane), S. negrosense, S. obscurum, S. occidentale, S. officinale, S. officinalis, S. officinarum (Cultivated sugar cane), S. officinarum ‘Chembon’, S. officinarum ‘Otaheite’, S. officinarum ‘Pele's Smoke’ (Black Magic Repellent Plant), S. officinarum L. ‘Laukona’, S. officinarum L. ‘Violaceum’, S. officinarum var. brevipedicellatum, S. officinarum var. officinarum, S. officinarum var. violaceum (Burgundy-Leaved sugar cane), S. pallidum, S. paniceum, S. panicosum, S. pappiferum, S. parviflorum, S. pedicellare, S. perrieri, S. polydactylum, S. polystachyon, S. polystachyum, S. porphyrocomum, S. procerum, S. propinquum, S. punctatum, S. rara, S. rarum, S. ravennae (Hardy Pampas Plume Grass), S. repens, S. reptans, S. ridleyi, S. robustum (Wild New-Guinean Cane), S. roseum, S. rubicundum, S. rufum, S. sagittatum, S. sanguineum, S. sape, S. sara, S. scindicus, S. semidecumbens, S. sibiricum, S. sikkimense, S. sinense (Cultivated sugar cane), S. sisca, S. sorghum, S. speciosissimum, S. sphacelatum, S. spicatum, S. spontaneum (Wild Sugar Cane), S. spontaneum var. insulare, S. spontanum, S. stenophyllum, S. stewartii, S. strictum, S. teneriffae, S. ternatum, S. thunbergii, S. tinctorium, S. tridentatum, S. trinii, S. tristachyum, S. velutinum, S. versicolor, S. viguieri, S. villosum, S. violaceum, S. wardii, S. warmingianwn, S. williamsii.

The term “plant part” as used herein includes a pod, root, sett root, shoot root, root primordial, shoot, primary shoot, secondary shoot, tassle, panicle, arrow, midrib, blade, ligule, auricle, dewlap, blade joint, sheath, node, internode, bud furrow, leaf scar, cutting, tuber, stem, stalk, fruit, berry, nut, flower, leaf, bark, wood, epidermis, vascular tissue, organ, protoplast, crown, callus culture, petiole, petal, sepal, stamen, stigma, style, bud, meristem, cambium, cortex, pith, sheath, silk, ovule or embryo. Other exemplary sugar cane plant parts are a meiocyte, gamete, ovule, pollen or endosperm of any of the plants of the invention. Other exemplary plant parts are a seed, seed-piece, embryo, protoplast, cell culture, any group of plant cells organized into a structural and functional unit, ratoon or propagule.

The term “promoter” is defined herein as a nucleic acid (e.g., DNA) sequence that allows the binding of RNA polymerase (including but not limited to RNA polymerase I, RNA polymerase II and/or RNA polymerase III from eukaryotes) and directs the polymerase to a downstream transcriptional start site of a nucleic acid sequence encoding a polypeptide to initiate transcription. RNA polymerase effectively catalyzes the assembly of messenger RNA complementary to the appropriate DNA strand of the coding region.

A “promoter operably linked to a heterologous gene” and similar terms is a promoter that is operably linked to a gene that is different from the gene to which the promoter is normally operably linked in its native state. Similarly, an “exogenous nucleic acid operably linked to a heterologous regulatory sequence” and similar terms is a nucleic acid that is operably linked to a regulatory control sequence to which it is not normally linked in its native state.

The term “hybrid promoter” is defined herein as parts of two or more promoters that are fused together to generate a sequence that is a fusion of the two or more promoters, which is operably linked to a coding sequence and mediates the transcription of the coding sequence into mRNA.

The term “tandem promoter” is defined herein as two or more promoter sequences each of which is operably linked to a coding sequence and mediates the transcription of the coding sequence into mRNA.

The term “constitutive active promoter” is defined herein as a promoter that allows stable expression of the gene of interest.

The term “inducible promoter” is defined herein as a promoter induced by the presence or absence of a biotic or an abiotic factor.

The term “polypeptide” does not refer to a specific length of the encoded product and, therefore, encompasses peptides, oligopeptides, and proteins. The term “exogenous polypeptide” is defined as a polypeptide which is not native to the plant cell, a native polypeptide in which modifications have been made to alter the native sequence and/or a native polypeptide whose expression is quantitatively altered as a result of a manipulation of the plant cell by recombinant DNA techniques.

As used herein, the term “pseudogene” refers to a non-functional copy of a protein-coding gene; pseudogenes found in the genomes of eukaryotic organisms are often inactivated by mutations and are thus presumed to be non-essential to that organism; pseudogenes of reverse transcriptase and other open reading frames found in retroelements are abundant in the centromeric regions of Arabidopsis and other organisms and are often present in complex clusters of related sequences.

In representative embodiments, the polynucleotides, nucleic acids, nucleotide sequences and centromere sequences of the invention are “recombinant.” As used herein, the term “recombinant” nucleic acid, polynucleotide, nucleotide sequence or centromere sequence refers to a nucleic acid, polynucleotide or nucleotide sequence that has been constructed, altered, rearranged and/or modified by genetic engineering techniques. The term “recombinant” does not refer to alterations that result from naturally occurring events, such as spontaneous mutations, or from non-spontaneous mutagenesis.

As used herein the term “regulatory sequence” refers to any nucleic acid (e.g., DNA) sequence that influences the efficiency of transcription or translation of any gene. The term includes, but is not limited to, sequences comprising promoters, enhancers, transcriptional initiation regions and/or terminators.

As used herein the term “repeated nucleotide sequence” and similar terms refer to any nucleic acid sequence of at least about 25 bp present in a genome or a recombinant molecule, other than a telomere repeat, that occurs at least two or more times and that are optionally substantially identical either in head to tail or head to head orientation either with or without intervening sequence between repeat units. Thus, the term “repeated nucleotide sequence” encompasses degenerate repeats that are not 100% identical, but are substantially identical either in head to tail or head to head orientation.

As used herein, the term “retroelement” or “retrotransposon” refers to a genetic element related to retroviruses that disperse through an RNA stage. The abundant retroelements present in plant genomes contain long terminal repeats (LTR retrotransposons) and encode a polyprotein gene that is processed into several proteins including a reverse transcriptase. Specific retroelements (complete or partial sequences) can be found in and around plant centromeres and can be present as dispersed copies or complex repeat clusters. Individual copies of retroelements may be truncated or contain mutations; intact retroelements are rarely encountered. In one embodiment, the retroelement is a CRS retroelement sequence (e.g., SEQ ID NO:74 or a fragment or other sequence derived therefrom).

As used herein the term “satellite DNA” refers to short DNA sequences (typically <1000 bp) present in a genome as multiple nucleotide repeats, mostly arranged in a tandemly repeated fashion, as opposed to a dispersed fashion. Repetitive arrays of specific satellite repeats are abundant in the centromeres of many higher eukaryotic organisms.

As used herein, a “screenable marker” is a gene whose presence results in an identifiable phenotype. This phenotype may be observable under standard conditions, altered conditions such as elevated temperature, or in the presence of certain chemicals used to detect the phenotype. The use of a screenable marker allows for the use of lower, sub-killing antibiotic concentrations and the use of a visible marker gene to identify clusters of transformed cells, and then manipulation of these cells to homogeneity. Illustrative screenable markers of the present include genes that encode fluorescent proteins that are detectable by a visual microscope such as the fluorescent reporter genes DsRed, ZsGreen, ZsYellow, AmCyan, Green Fluorescent Protein (GFP) and modifications of these reporter genes to excite or emit at altered wavelengths. An additional screenable marker gene is lac.

Alternative methods of screening for modified plant cells may involve use of relatively low, sub-killing concentrations of a selection agent (e.g. sub-killing antibiotic concentrations), and also involve use of a screenable marker (e.g., a visible marker gene) to identify clusters of modified cells carrying the screenable marker, after which these screenable cells are manipulated to homogeneity. As used herein, a “selectable marker” is a gene whose presence results in a clear phenotype, and most often a growth advantage for cells that contain the marker. This growth advantage may be present under standard conditions, altered conditions such as elevated temperature, specialized media compositions, or in the presence of certain chemicals such as herbicides or antibiotics. Use of selectable markers is described, for example, in Broach et al. Gene, 8:121-133, 1979. Examples of selectable markers include the thymidine kinase gene, the cellular adenine phosphoribosyltransferase gene and the dihydrylfolate reductase gene, hygromycin phosphotransferase genes, the bar gene, neomycin phosphotransferase genes and phosphomannose isomerase gene, among others. Nonlimiting examples of selectable markers in the present invention include genes whose expression confer antibiotic or herbicide resistance to the host cell, or proteins allowing utilization of a carbon source not normally utilized by plant cells. Expression of one of these markers should be sufficient to enable the survival of those cells that comprise a vector within the host cell, and facilitate the manipulation of the vector into new host cells. Of particular interest in the present invention are proteins conferring cellular resistance to kanamycin, G 418, paramomycin, hygromycin, bialaphos, and glyphosate for example, or proteins allowing utilization of a carbon source, such as mannose, not normally utilized by plant cells.

The term “stable” as used herein means that the mini-chromosome can be transmitted to at least some daughter cells over at least about 1, 2, 3, 4, 5, 6, 7, 8 or more mitotic generations. Some embodiments of mini-chromosomes may be transmitted as functional, autonomous units over at least about 1, 2, 3, 4, 5, 6, 7, 8 or more generations. According to representative embodiments, the mini-chromosome can be transmitted over at least about 8, 9, 10, 11, 12, 13, 14, 15, 16, 17, 18, 18, 19, 20, 21, 22, 23, 24, 25, 26, 27, 28, 29 or 30 generations, for example, through the regeneration or differentiation of an entire plant, and may even be transmitted through meiotic division to gametes. Other representative mini-chromosomes can be further maintained in the zygote derived from such a gamete or in an embryo or endosperm derived from one or more such gametes. A “functional and stable” mini-chromosome is one in which functional mini-chromosomes can be detected in at least some daughter cells after transmission of the mini-chromosomes over at least about 1, 2, 3, 4, 5, 6, 7, 8 or more mitotic generations and/or after inheritance through a meiotic division. During mitotic division, as occurs occasionally with native chromosomes, there may be some non-transmission of mini-chromosomes; the mini-chromosome may still be characterized as stable despite the occurrence of such events if an adchromosomal plant that contains descendants of the mini-chromosome distributed throughout its parts may be regenerated from cells, cuttings, propagules, or cell cultures containing the mini-chromosome, or if an adchromosomal plant can be identified in progeny of the plant containing the mini-chromosome.

As used herein, a “structural gene” is a sequence which codes for a polypeptide or functional untranslated RNA and includes 5′ and 3′ ends. The structural gene may be from the host into which the structural gene is transformed or from another species. A structural gene may optionally, but not necessarily, include one or more regulatory sequences which modulate the expression of the structural gene, such as a promoter, terminator or enhancer. A structural gene may optionally, but not necessarily, confer some useful phenotype upon an organism comprising the structural gene, for example, herbicide resistance. In one embodiment of the invention, a structural gene may encode a functional RNA sequence which is not translated into a protein, for example a tRNA or rRNA gene.

Two nucleotide sequences are “substantially identical” or share “substantial identity” if the nucleotide sequences are at least about 70%, 75%, 80%, 85%, 90%, 95%, 97%, 98%, 99% or more identical. In representative embodiments, “substantially identical” encompasses ranges of at least about 84% identical (e.g., to SEQ ID NO:1); at least about 74% identical (e.g., to SEQ ID NO:2), at least about 73% identical (e.g., to SEQ ID NO:3), at least about 77% identical (e.g., to SEQ ID NO:4), at least about 72% identical (e.g., to SEQ ID NO:5), at least about 74% identical (e.g., to SEQ ID NO:6), at least about 75% identical (e.g., to SEQ ID NO:7), at least about 75% identical (e.g., to SEQ ID NO:8); at least about 74% identical (e.g., to SEQ ID NO:9), at least about 75% identical (e.g., to SEQ ID NO:10), at least about 76% identical (e.g., to SEQ ID NO:11), at least about 80% identical (e.g., to SEQ ID NO:12), from about 84% to 92% identical (e.g., to SEQ ID NO:1), from about 74% to 100% identical (e.g., to SEQ ID NO:2), from about 73% to 92% identical (e.g., to SEQ ID NO:3), from about 77% to 92% identical (e.g., to SEQ ID NO:4), from about 72% to 100% identical (e.g., to SEQ ID NO:5), from about 74% to 100% identical (e.g., to SEQ ID NO:6), from about 75% to 100% identical (e.g., to SEQ ID NO:7), from about 75% to 88% identical (e.g., to SEQ ID NO:8); from about 74% to 93% identical (e.g., to SEQ ID NO:9), from about 75% to 100% identical (e.g., to SEQ ID NO:10), or from about 76% to 93% identical (e.g., to SEQ ID NO:11), or from about 80% to 90% identical (e.g., to SEQ ID NO:12). Those skilled in the art will appreciate that native centromere sequences generally comprise degenerate repeat sequences that are not 100% identical.

The invention specifically contemplates the alternative use of fragments or variants (mutants) of any of the nucleic acids described herein that retain the desired activity, including nucleic acids that function as sugar cane centromeres, nucleic acids that function as promoters or other regulatory control sequences, or exogenous nucleic acids. Variants may have one or more additions, substitutions and/or deletions of nucleotides within the original nucleotide sequence or consensus sequence. Variants include nucleic acid sequences that are at least substantially identical to the original nucleic acid sequence. Variants also include nucleic acid sequences that hybridize under low, medium, high or very high stringency conditions to the original nucleic acid sequence. Similarly, the invention also contemplates the alternative use of fragments or variants of any of the polypeptides described herein.

As is known in the art, a number of different mathematical algorithms and programs can be used to determine the degree of sequence identity between two nucleotide sequences. For example, the percent identity between two nucleotide sequences can be determined using the Needleman and Wunsch (1970) J. Mol. Biol. 48:444-453 algorithm that has been incorporated into the GAP program in the GCG software package (available at www.gcg.com), using either a Blossum 62 matrix or a PAM250 matrix. The parameters can be set so as to maximize the percent identity. Other methods for determining sequence identity are described herein (see, e.g., the Examples).

As used herein, the term “telomere” or “telomere DNA” refers to a sequence capable of capping the ends of a chromosome, thereby preventing degradation of the chromosome end, ensuring replication and preventing fusion to other chromosome sequences. Telomeres can include naturally occurring telomere sequences or synthetic sequences. Telomeres from one species may confer telomere activity in another species. An exemplary telomere DNA is a heptanucleotide telomere repeat TTTAGGG (and its complement) found in the majority of plants.

“Transformed,” “transgenic,” “modified,” and “recombinant” refer to a host organism such as a plant into which an exogenous or heterologous nucleic acid molecule has been introduced, and includes meiocytes, seeds, zygotes, embryos, endosperm, or progeny of such plant that retain the exogenous or heterologous nucleic acid molecule but which have not themselves been subjected to the transformation process.

When the phrase “transmission efficiency” of a certain percent is used, transmission percent efficiency is calculated by measuring mini-chromosome presence through one or more mitotic and/or meiotic generations. For example, it can be directly measured as the ratio (expressed as a percentage) of the daughter cells or plants demonstrating presence of the mini-chromosome to parental cells or plants demonstrating presence of the mini-chromosome. Presence of the mini-chromosome in parental and daughter cells can be demonstrated with assays that detect the presence of an exogenous nucleic acid carried on the mini-chromosome. Exemplary assays include the detection of a screenable marker (e.g. presence of a fluorescent protein or any gene whose expression results in an observable phenotype), a selectable marker, or PCR amplification of any exogenous nucleic acid carried on the mini-chromosome.

### I. Sugar Cane Centromeres, Mini-Chromosomes and Sugar Cane Plants, Plant Parts, Plant Tissues and Cells Comprising the Same.

The invention provides sugar cane mini-chromosomes, which may be functional, stable, autonomous sugar cane mini-chromosomes, comprising centromeres comprising sugar cane repeat sequences including native and/or synthetic sequences. Optionally, the sugar cane mini-chromosome is “isolated.” The invention also provides for “adchromosomal” sugar cane plants as described in further detail herein.

One aspect of the invention is related to sugar cane plants comprising one or more sugar cane mini-chromosomes of the invention, optionally comprising one or more exogenous nucleic acids (including extra copies of a nucleic acid that already exists in the sugar cane genome). In representative embodiments, the mini-chromosome is autonomous. Such plants comprising autonomous sugar cane mini-chromosomes are contrasted with transgenic plants whose genome has been altered by integrating exogenous nucleic acid transgenes into the native sugar cane chromosomes. In representative embodiments, expression of the exogenous nucleic acid, either constitutively or in response to a signal (which may be induced by challenge or a stimulus) and/or tissue specific expression and/or time specific expression, results in an altered phenotype of the plant.

In particular embodiments, the invention provides for sugar cane mini-chromosomes comprising at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 250, 500, 1000 or more exogenous nucleic acids.

The invention contemplates that sugar cane plants may be used to carry the sugar cane mini-chromosomes as described herein, either autonomously and/or in integrated form. A related aspect of the invention is a sugar cane plant part or plant tissue, including a pod, root, sett root, shoot root, root primordial, shoot, primary shoot, secondary shoot, tassle, panicle, arrow, midrib, blade, ligule, auricle, dewlap, blade joint, sheath, node, internode, bud furrow, leaf scar, cutting, tuber, stem, stalk, fruit, berry, nut, flower, leaf, bark, wood, epidermis, vascular tissue, organ, protoplast, crown, callus culture, petiole, petal, sepal, stamen, stigma, style, bud, meristem, cambium, cortex, pith, sheath, silk, ovule or embryo. Other exemplary sugar cane plant parts are a meiocyte, gamete, ovule, pollen or endosperm of any of the plants of the invention. Other exemplary plant parts are a seed, seed-piece, embryo, protoplast, cell culture, any group of plant cells organized into a structural and functional unit, ratoon or propagule of any of the sugar cane plants of the invention.

In one embodiment, the exogenous nucleic acid is primarily expressed in a specific or—preferred location or tissue of a sugar cane plant, for example, stem, epidermis, vascular tissue, meristem, cambium, cortex, pith, leaf, sheath, flower, root or seed. Tissue-specific or—preferred expression can be accomplished with, for example, localized presence of the sugar cane mini-chromosome, selective maintenance of the sugar cane mini-chromosome, or with promoters that drive tissue-specific or—preferred expression.

Another related aspect of the invention is sugar cane meiocytes, pollen, ovules, endosperm, seed, somatic embryos, apomyctic embryos, embryos derived from fertilization, vegetative propagules and progeny of the originally adchromosomal plant and of its filial generations that retain the sugar cane mini-chromosome, optionally autonomously. Such progeny include clonally propagated sugar cane plants, embryos and plant parts as well as filial progeny from self- and cross-breeding, and from apomyxis.

In representative embodiments, the sugar cane mini-chromosome is transmitted to subsequent generations of viable daughter cells during mitotic cell division with a transmission efficiency of at least about 60%, 70%, 80%, 85%, 90%, 95%, 96%, 97%, 98%, or 99%.

In embodiments of the invention, during meiotic division, the sugar cane mini-chromosome is transmitted to viable gametes with a transmission efficiency of at least about 60%, 70%, 80%, 85%, 90%, 95%, 96%, 97%, 98%, or 99% when more than one copy of the sugar cane mini-chromosome is present in the gamete mother cell(s) of the plant. The sugar cane mini-chromosome can optionally be transmitted to viable gametes during meiotic cell division with a transmission frequency of at least about 1%, 10%, 20%, 30%, 40%, 45%, 46%, 47%, 48%, or 49% when one copy of the mini-chromosome is present in the gamete mother cell(s) of the sugar cane plant. According to embodiments of the invention, for production of seeds via sexual reproduction or by apomyxis the sugar cane mini-chromosome is transferred into at least about 60%, 70%, 80%, 85%, 90%, 95%, 96%, 97%, 98%, or 99% of viable embryos when at least one cell of the plant contains more than one copy of the sugar cane mini-chromosome. For production of seeds via sexual reproduction or by apomyxis from sugar cane plants with one mini-chromosome per cell, the sugar cane mini-chromosome is optionally transferred into at least about 1%, 10%, 20%, 30%, 40%, 45%, 46%, 47%, 48%, or 49% of viable embryos.

In representative embodiments of the invention, a sugar cane mini-chromosome that comprises an exogenous selectable trait or exogenous selectable marker can be employed to increase the frequency in subsequent generations of adchromosomal cells, tissues, gametes, embryos, endosperm, seeds, plants or progeny that comprise the sugar cane minichromosome. In particular embodiments, the frequency of transmission of sugar cane mini-chromosomes into viable cells, tissues, gametes, embryos, endosperm, seeds, plants or progeny can be at least about 90%, 92%, 95%, 96%, 97%, 98%, 99% or 99.5% after mitosis or meiosis by applying at least one selection that favors the survival of adchromosomal cells, tissues, gametes, embryos, endosperm, seeds, plants or progeny over such cells, tissues, gametes, embryos, endosperm, seeds, plants or progeny lacking the mini-chromosome.

Transmission efficiency may be measured as the percentage of sugar cane progeny cells or sugar cane plants that carry the sugar cane mini-chromosome as measured by one of several assays taught herein including detection of reporter gene fluorescence, PCR detection of a sequence that is carried by the mini-chromosome, RT-PCR detection of a gene transcript for a gene carried on the sugar cane mini-chromosome, Western analysis of a protein produced by a gene carried on the sugar cane mini-chromosome, Southern analysis of the DNA (either in total or a portion thereof) carried by the sugar cane mini-chromosome, fluorescence in situ hybridization (FISH) or in situ localization by repressor binding, to name a few. Any assay used to detect the presence of the sugar cane mini-chromosome (or a portion of the mini-chromosome) may be used to measure the efficiency that a parental cell or plant transmits the mini-chromosome to its progeny. Efficient transmission as measured by some benchmark percentage should indicate the degree to which the sugar cane mini-chromosome is stable through the mitotic and meiotic cycles.

Sugar cane plants of the invention may also contain chromosomally integrated exogenous nucleic acid in addition to an autonomous sugar cane mini-chromosome(s) of the invention. The modified sugar cane plants or plant parts, including plant tissues of the invention may include sugar cane plants that have chromosomal integration of some portion of the mini-chromosome (e.g. exogenous nucleic acid or centromere sequences) in some or all cells of the plant. In one aspect of the invention, an autonomous sugar cane mini-chromosome can be isolated from integrated exogenous nucleic acid by crossing the modified sugar cane plant containing the integrated exogenous nucleic acid with sugar cane plants producing some gametes lacking the integrated exogenous nucleic acid and subsequently isolating offspring of the cross, or subsequent crosses, that are modified but lack the integrated exogenous nucleic acid. This independent segregation of the sugar cane mini-chromosome is one measure of the autonomous nature of the mini-chromosome.

Another aspect of the invention relates to methods for producing and, optionally, isolating such modified sugar cane plants containing a sugar cane mini-chromosome of the invention, optionally a functional, stable, autonomous sugar cane mini-chromosome.

In one embodiment, the invention contemplates improved methods for isolating native sugar cane centromere sequences. In another embodiment, the invention contemplates methods for generating variants of native or artificial sugar cane centromere sequences by passage through other host cells such as bacterial or fungal hosts.

In a further embodiment, the invention contemplates methods for delivering a sugar cane mini-chromosome into sugar cane plant cells or tissues to transform the cells or tissues, optionally detecting mini-chromosome presence and/or assessing mini-chromosome performance, and optionally generating a sugar cane plant from such cells or tissues.

Exemplary assays for assessing sugar cane mini-chromosome performance include lineage-based inheritance assays, use of chromosome loss agents to demonstrate autonomy, exonuclease digestion, global mitotic mini-chromosome inheritance assays (sectoring assays) with or without the use of agents inducing chromosomal loss, assays measuring expression levels of genes (including marker genes) carried by the sugar cane mini-chromosome over time and space in a sugar cane plant, physical assays for separation of autonomous sugar cane mini-chromosomes from endogenous nuclear chromosomes of sugar cane plants, molecular assays demonstrating conserved sugar cane mini-chromosome structure, such as PCR, Southern blots, sugar cane mini-chromosome rescue, cloning and characterization of sugar cane mini-chromosome sequences present in the sugar cane plant, cytological assays detecting sugar cane mini-chromosome presence in the sugar cane cell's genome (e.g. FISH) and/or meiotic sugar cane mini-chromosome inheritance assays, which measure the levels of mini-chromosome inheritance into a subsequent generation of sugar cane plants via meiosis and gametes, embryos, endosperm or seeds.

Another aspect of the invention relates to methods for using sugar cane plants containing a sugar cane mini-chromosome(s) for producing food or feed products, pharmaceutical products, biofuels and chemical products by appropriate expression of exogenous nucleic acid(s) contained within the mini-chromosome(s).

Yet another aspect of the invention provides novel sugar cane mini-chromosomes (e.g., autonomous mini-chromosomes) with novel compositions and structures, that are used to transform plant cells which are in turn used to generate a plant (or multiple plants).

In a related aspect, novel sugar cane centromere compositions characterized by sequence content, size and/or other parameters are provided as described herein. Optionally, the minimal size of sugar cane centromeric sequence is utilized in mini-chromosome construction. The sugar cane centromeric nucleic acid segment can be derived from a portion of sugar cane genomic DNA and/or synthesized based on a sugar cane satellite repeat sequence.

Another related aspect is the novel structure of the sugar cane mini-chromosome, particularly structures lacking bacterial sequences (e.g., sequences required for bacterial propagation), referred to as backbone-free sugar cane mini-chromosomes.

In other exemplary embodiments, the invention contemplates sugar cane mini-chromosomes or other vectors comprising centromeric nucleotide sequence that when hybridized to 1, 2, 3, 4, 5, 6, 7, 8 or more of the probes described herein, under hybridization conditions described herein, e.g. low, medium or high stringency, provides relative hybridization scores. Modified or adchromosomal sugar cane plants or plant parts containing such sugar cane mini-chromosomes are contemplated.

The advantages of the present invention include without limitation: provision of an autonomous, independent genetic linkage group for accelerating sugar cane breeding; lack of disruption of host sugar cane genome; multiple gene “stacking” of large and potentially unlimited numbers of genes; uniform genetic composition of exogenous DNA sequences in plant cells and plants containing autonomous sugar cane mini-chromosomes; defined genetic context for predictable gene expression; and higher frequency occurrence and recovery of sugar cane plant cells and plants containing stably maintained exogenous DNA due to elimination of an inefficient integration step. In addition, sugar cane mini-chromosomes that increase total recoverable sugars and/or enhance the utility of modified sugar cane plants for use in biofuel production are specifically envisioned.

### II. Composition of Mini-Chromosomes and Mini-Chromosome Construction

The sugar cane mini-chromosomes of the present invention may contain a variety of elements, including without limitation: (1) sequences that function as sugar cane centromeres, (2) optionally, one or more exogenous nucleic acids, including, for example, plant-expressed nucleic acids, or nucleic acids encoding functional, untranslated RNAs, (3) optionally, sequences that function as an origin of replication, which may be included in the region that functions as a plant centromere, (4) optionally, a bacterial plasmid backbone for propagation of the plasmid in bacteria, (5) optionally, sequences that function as plant telomeres, (6) optionally, additional “stuffer DNA” sequences that serve to physically separate the various components on the sugar cane mini-chromosome from each other, (7) optionally “buffer” sequences such as MARs (Matrix Attachment Regions) or SARs (Scaffold Attachment Regions), (8) optionally marker sequences of any origin, including but not limited to plant or bacterial origin, (9) optionally, sequences that serve as recombination sites, and/or (10) optionally, “chromatin packaging sequences” such as cohesion and condensing binding sites. In representative embodiments, the mini-chromosome comprises a plurality of restriction sites for insertion of one or more exogenous nucleic into the mini-chromosome.

The sugar cane mini-chromosomes of the present invention may be constructed to include various components which are novel, which include, but are not limited to, a sugar cane centromere of the invention, as described in further detail herein.

### Sugar Cane Mini-Chromosome Sequence Content and Structure

Sugar cane-expressed genes from non-plant sources may be modified to accommodate sugar cane codon usage, to insert preferred motifs near the translation initiation ATG codon, to remove sequences recognized in plants as 5′ or 3′ splice sites and/or to better reflect plant GC/AT content. Plant genes typically have a GC content of more than 35%, and coding sequences which are rich in A and T nucleotides can be problematic. For example, ATTTA motifs may destabilize mRNA; plant polyadenylation signals such as AATAAA at inappropriate positions within the message may cause premature truncation of transcription; and monocotyledons such as sugar cane may recognize AT-rich sequences as splice sites.

Each exogenous nucleic acid or sugar cane-expressed gene may optionally include a promoter, a coding region and a terminator sequence, which may be separated from each other by restriction endonuclease sites or recombination sites or both. The exogenous nucleic acid may also include introns, which may be present in any number and at any position within the transcribed portion of the nucleic acid, including the 5′ untranslated sequence, the coding region and the 3′ untranslated sequence. Introns may be natural plant introns derived from any plant, or artificial introns based on the splice site consensus that has been defined for plant species. Some intron sequences have been shown to enhance expression in plants. Optionally the exogenous nucleic acid may include a plant transcriptional terminator, non-translated leader sequences derived from viruses that enhance expression, a minimal promoter, or a signal sequence controlling the targeting of gene products to plant compartments or organelles.

The coding regions of the nucleic acids can encode any protein, including but not limited to visible marker genes (for example, fluorescent protein genes, other genes conferring a visible phenotype to the plant) or other screenable or selectable markers (for example, conferring resistance to antibiotics, herbicides or other toxic compounds or encoding a protein that confers a growth advantage to the cell expressing the protein) or nucleic acids that confer some commercial or agronomic value to the modified or adchromosomal sugar cane plant. Multiple nucleic acids can be placed on the same sugar cane mini-chromosome vector. The nucleic acids may be separated from each other by restriction endonuclease sites, homing endonuclease sites, recombination sites or any combinations thereof. Alternatively, the cloning process can be executed in a manner that destroys the intervening restriction sites. Any number of nucleic acids can be present.

Nucleic acids comprising a sugar cane mini-chromosome of the invention may also contain vector sequences such as a bacterial plasmid backbone for propagation of the nucleic acid in bacteria such as E. coli, A. tumefaciens, or A. rhizogenes. The plasmid backbone may be that of a low-copy vector or in other embodiments it may be desirable to use a mid to high level copy backbone. In one embodiment of the invention, this backbone contains the replicon of the F′ plasmid of E. coli. However, other plasmid replicons, such as the bacteriophage P1 replicon, or other low-copy plasmid systems such as the RK2 replication origin, may also be used. The backbone may include one or several antibiotic-resistance genes conferring resistance to a specific antibiotic to the bacterial cell in which the plasmid is present. Bacterial antibiotic-resistance genes include but are not limited to kanamycin-, ampicillin-, chloramphenicol-, streptomycin-, spectinomycin-, tetracycline- and gentamycin-resistance genes.

The sugar cane mini-chromosome may also contain plant telomeres. Telomeric sequences are known in the art. An exemplary telomere sequence is TTTAGGG or its complement. Telomeres are specialized DNA structures at the ends of linear chromosomes that function to stabilize the ends and facilitate the complete replication of the extreme termini of the DNA molecule (Richards et al., Cell, 1988 Apr. 8; 53(1):127-36; Ausubel et al., Current Protocols in Molecular Biology, Wiley & Sons, 1997).

Additionally, the sugar cane mini-chromosome may contain “stuffer DNA” sequences that serve to separate the various components on the mini-chromosome (centromere, genes, telomeres) from each other.

In one embodiment of the invention, the sugar cane mini-chromosome has a circular structure without telomeres. In another embodiment, the sugar cane mini-chromosome has a circular structure with telomeres. In a third embodiment, the sugar cane mini-chromosome has a linear structure with telomeres, for example, as would result if a “linear” structure were to be cut with a unique endonuclease, exposing the telomeres at the ends of a DNA molecule that contains all of the sequence contained in the original, closed construct with the exception of an antibiotic-resistance gene. In a fourth embodiment of the invention, the telomeres are placed in such a manner that the bacterial replicon, backbone sequences, antibiotic-resistance genes and any other sequences of bacterial origin and present for the purposes of propagation of the sugar cane mini-chromosome in bacteria, can be removed from the plant-expressed genes, the centromere, telomeres, and other sequences by cutting the structure with, for example, a unique endonuclease. This results in a sugar cane mini-chromosome from which much of, or even all, bacterial sequences have been removed. In this embodiment, bacterial sequence present between or among the plant-expressed genes or other sugar cane mini-chromosome sequences are excised prior to removal of the remaining bacterial sequences by cutting the sugar cane mini-chromosome with an endonuclease and re-ligating the structure such that the antibiotic-resistance gene has been lost. The unique endonuclease site may be the recognition sequence of any of a number of endonucleases including but not limited to restriction endonucleases, meganucleases, or homing endonuclease. Alternatively, the endonucleases and their sites can be replaced with any specific DNA cutting mechanism and its specific recognition site such as rare-cutting endonuclease or recombinase and its specific recognition site, as long as that site is present in the sugar cane mini-chromosomes only at the indicated positions.

Various structural configurations are possible by which sugar cane mini-chromosome elements can be oriented with respect to each other. In representative embodiments, a sugar cane centromere can be placed on a sugar cane mini-chromosome either between exogenous nucleic acids or outside a cluster of exogenous nucleic acids next to one telomere or next to the other telomere. Stuffer DNAs can be combined with these configurations to place the stuffer sequences inside the telomeres, around the centromere between genes or any combination thereof. Thus, a large number of alternative sugar cane mini-chromosome structures are possible, depending on the relative placement of the centromere, exogenous nucleic acids, stuffer DNAs, bacterial sequences, telomeres, and other sequences. The sequence content of each of these variants is the same, but their structure may be different depending on how the sequences are placed. These variations in architecture are possible both for linear and for circular sugar cane mini-chromosomes.

Furthermore, in representative embodiments, the mini-chromosome comprises a plurality of restriction sites for insertion of one or more exogenous nucleic into the mini-chromosome.

### Exemplary Centromere Components

The centromere in the mini-chromosome of the present invention may comprise novel sugar cane genomic and/or synthetic repeating centromeric sequences.

Centromeres comprising one, two, three, four, five, six, seven, eight, nine, ten, 15, 20 or more of the elements contained in any of the exemplary centromeres described in the examples below are also contemplated.

As described elsewhere herein, the invention specifically contemplates the alternative use of fragments or variants (mutants) of any of the sugar cane centromeres described herein that retain the desired activity.

Sugar cane centromere components may be isolated or derived from a native plant genome, for example, modified through recombinant techniques or through the cell-based techniques described herein. Alternatively, wholly artificial centromere components may be constructed using as a general guide the sequence of native sugar cane centromeres such as native satellite repeat sequences. Combinations of centromere components derived from natural sources and/or combinations of naturally derived and artificial components are also contemplated.

In one embodiment, the sugar cane centromere contains n copies of a repeated nucleotide sequence as described herein; wherein n is at least 2. In another embodiment, the sugar cane centromere contains n copies of interdigitated repeats. An interdigitated repeat is a DNA sequence that consists of two distinct repetitive elements that combine to create a unique permutation. Potentially any number of repeat copies capable of physically being placed on the construct can be included, including about 5, 10, 15, 20, 30, 50, 75, 100, 150, 200, 300, 400, 500, 750, 1,000, 1,500, 2,000, 3,000, 5,000, 7,500, 10,000, 20,000, 30,000, 40,000, 50,000, 60,000, 70,000, 80,000, 90,000 and about 100,000, including all ranges in-between such copy numbers. Moreover, the copies, while substantially identical, can vary from each other (i.e., can be degenerate). Such repeat variation is commonly observed in naturally occurring centromeres. The length of the repeat may vary, and can range from about 20 bp to about 360 bp, from about 20 bp to about 250 bp, from about 50 bp to about 225 bp, from 20 bp to 137 bp, from about 75 bp to about 210 bp, from about 100 bp to about 205 bp, from about 125 bp to about 200 bp, from about 150 bp to about 195 bp, from about 160 bp to about 190 and from about 170 bp to about 185 bp including about 180 bp.

The invention contemplates that two or more sugar cane repeated nucleotide sequences may be oriented head to tail within the centromere. The term “head to tail” refers to multiple consecutive copies of the same or substantially identical repeated nucleotide sequence (e.g., at least about 70% identical) that are in the same 5′-3′ orientation. The invention also contemplates that two or more of these repeated nucleotide sequences may be consecutive within the sugar cane centromere. The term “consecutive” refers to the same or substantially identical repeated nucleotide sequences (e.g., at least about 70% identical) that follow one after another without being interrupted by other significant sequence elements. Such consecutive repeated nucleotide sequences may be in any orientation, e.g. head to tail, tail to tail, or head to head, and may be separated by n number of nucleotides, wherein n ranges from 1 to 10, or 1 to 20, or 1 to 30, or 1 to 40, or 1 to 50. Exemplary repeated nucleotide sequences derived from sugar cane are set out as SEQ ID NOS:1-72, SEQ ID NO:73, and CRS (SEQ ID NO:74) and sequences that are substantially identical to any of the foregoing and/or hybridize to any of the foregoing under stringent hybridization conditions.

### Exemplary Exogenous Nucleic Acids Including Plant-Expressed Genes

Of particular interest in the present invention are exogenous nucleic acids which when introduced into sugar cane plants will alter the phenotype of the plant, a plant organ, plant tissue, or a portion of the plant. Exemplary exogenous nucleic acids encode polypeptides. Other exemplary exogenous nucleic acids alter expression of exogenous or endogenous genes, either increasing or decreasing expression, optionally in response to a specific signal or stimulus. Still further, exogenous nucleic acids can encode functional, untranslated RNAs, the expression of which may result in an altered phenotype.

As used herein, the term “trait” can refer either to the altered phenotype of interest or the nucleic acid which causes the altered phenotype of interest.

One of the purposes of transformation of sugar cane is to add some commercially desirable, agronomically important traits to the plant. Such traits include, but are not limited to, enhanced production of total recoverable sugars; utility for production of biofuels; herbicide resistance and/or tolerance, insect (pest) resistance and/or tolerance; disease resistance and/or tolerance (viral, bacterial, fungal, nematode or other pathogens); stress tolerance and/or resistance, as exemplified by resistance or tolerance to drought, heat, chilling, freezing, excessive moisture, salt stress, mechanical stress, extreme acidity, alkalinity, toxins, UV light, ionizing radiation and/or oxidative stress; increased yields, increased biomass, whether in quantity and/or quality; enhanced and/or altered nutrient acquisition and enhanced and/or altered metabolic efficiency; enhanced and/or altered nutritional content and makeup of plant tissues used for food, feed, fiber and/or processing; physical appearance; male sterility; drydown; standability; prolificacy; starch quantity and/or quality; oil quantity and/or quality; protein quality and/or quantity; amino acid composition; modified chemical production; altered pharmaceutical and/or nutraceutical properties; altered bioremediation properties; increased biomass; altered growth rate; altered fitness; altered biodegradability; altered CO₂ fixation; presence of bioindicator activity; altered digestibility by humans and/or animals; altered allergenicity; altered mating characteristics; altered pollen dispersal; improved environmental impact; altered nitrogen fixation capability; the production of a pharmaceutically active protein; the production of a small molecule with medicinal properties; the production of a chemical including those with industrial utility; the production of nutraceuticals, food and/or animal feed additives, carbohydrates, RNAs, lipids, fuels, dyes, pigments, vitamins, scents, flavors, vaccines, antibodies, hormones, and the like; and/or alterations in plant architecture and/or development, including changes in developmental timing, photosynthesis, signal transduction, cell growth, reproduction, and/or differentiation.

Additionally one could create a library of an entire genome (or a portion thereof) from any organism or organelle including mammals, plants, microbes, fungi, or bacteria, represented on sugar cane mini-chromosomes.

In one embodiment, the sugar cane plant comprising a sugar cane mini-chromosome may exhibit increased or decreased expression and/or accumulation of a product of the plant, which may be a natural product of the plant or a new or altered product of the plant. Exemplary products include an enzyme, an RNA molecule, a nutritional protein, a structural protein, an amino acid, a lipid, a fatty acid, a polysaccharide, a sugar, an alcohol, an alkaloid, a carotenoid, a propanoid, a phenylpropanoid, or terpenoid, a steroid, a flavonoid, a phenolic compound, an anthocyanin, a pigment, a vitamin or a plant hormone. In another embodiment, the sugar cane plant comprising a sugar cane mini-chromosome has enhanced or diminished requirements for light, water, nitrogen, and/or trace elements. In another embodiment the sugar cane plant comprising a sugar cane mini-chromosome has an enhanced ability to capture and/or fix nitrogen from its environment. In yet another embodiment, the sugar cane plant comprising a sugar cane mini-chromosome is enriched for an essential amino acid as a proportion of a protein fraction of the plant. The protein fraction may be, for example, total seed protein, soluble protein, insoluble protein, water-extractable protein, and/or lipid-associated protein. The sugar cane mini-chromosome may include genes that cause the overexpression, underexpression, antisense modulation, sense suppression, inducible expression, inducible repression, and/or inducible modulation of another gene.

A brief summary of exemplary improved properties and polypeptides of interest for either increased or decreased expression is provided below.

(i) Herbicide Resistance

An herbicide resistance (or tolerance) trait is a characteristic of a sugar cane plant comprising a sugar cane mini-chromosome that is resistant to dosages of an herbicide that is typically lethal to a wild type plant. Exemplary herbicides for which resistance is useful in a plant include glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides and glufosinate herbicides. Other herbicides would be useful as would combinations of herbicide genes on the same sugar cane mini-chromosome.

The genes encoding phosphinothricin acetyltransferase (bar), glyphosate tolerant EPSP synthase genes, glyphosate acetyltransferase, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are good examples of herbicide resistant genes for use in transformation. The bar gene codes for an enzyme, phosphinothricin acetyltransferase (PAT), which inactivates the herbicide phosphinothricin and prevents this compound from inhibiting glutamine synthetase enzymes. The enzyme 5 enolpyruvylshikimate 3 phosphate synthase (EPSP Synthase), is normally inhibited by the herbicide N (phosphonomethyl)glycine (glyphosate). However, genes are known that encode glyphosate resistant EPSP synthase enzymes. These genes are particularly contemplated for use in plant transformation. The deh gene encodes the enzyme dalapon dehalogenase and confers resistance to the herbicide dalapon. The bxn gene codes for a specific nitrilase enzyme that converts bromoxynil to a non herbicidal degradation product. The glyphosate acetyl transferase gene inactivates the herbicide glyphosate and prevents this compound from inhibiting EPSP synthase.

Polypeptides that may produce plants having tolerance to plant herbicides include polypeptides involved in the shikimate pathway, which are of interest for providing glyphosate tolerant plants. Such polypeptides include polypeptides involved in biosynthesis of chorismate, phenylalanine, tyrosine and tryptophan.

(ii) Insect Resistance

Potential insect resistance (or tolerance) genes that can be introduced include Bacillus thuringiensis toxin genes or Bt genes (Watrud et al., In: Engineered Organisms and the Environment, 1985). Bt genes may provide resistance to lepidopteran or coleopteran pests such as European Corn Borer (ECB). Exemplary Bt toxin genes for use in such embodiments include the CryIA(b) and CryIA(c) genes. Endotoxin genes from other species of B. thuringiensis which affect insect growth or development also may be employed in this regard.

It is contemplated that in some embodiments Bt genes for use in the sugar cane mini-chromosomes disclosed herein will be those in which the coding sequence has been modified to effect increased expression in plants, and for example, in monocot plants including sugar cane. Means for preparing synthetic genes are well known in the art and are disclosed in, for example, U.S. Pat. No. 5,500,365 and U.S. Pat. No. 5,689,052.

Examples of such modified Bt toxin genes include a synthetic Bt CryIA(b) gene (Perlak et al., Proc. Natl. Acad. Sci. USA, 88:3324-3328, 1991), and the synthetic CryIA(c) gene termed 1800b (PCT Application WO 95/06128).

Protease inhibitors also may provide insect resistance (Johnson et al., Proc Natl Acad Sci USA. 1989 December; 86(24): 9871-9875), and will thus have utility in sugar cane transformation. The use of a pinII gene in combination with a Bt toxin gene, the combined effect of which has been discovered to produce synergistic insecticidal activity is envisioned to be particularly useful. Other genes which encode inhibitors of the insect's digestive system, or those that encode enzymes or co factors that facilitate the production of inhibitors, also may be useful. This group may be exemplified by oryzacystatin and amylase inhibitors such as those from wheat and barley.

Amylase inhibitors are found in various plant species and are used to ward off insect predation via inhibition of the digestive amylases of attacking insects. Several amylase inhibitor genes have been isolated from plants and some have been introduced as exogenous nucleic acids, conferring an insect resistant phenotype that is potentially useful (“Plants, Genes, and Crop Biotechnology” by Maarten J. Chrispeels and David E. Sadava (2003) Jones and Bartlett Press).

Genes encoding lectins may confer additional or alternative insecticide properties. Lectins are multivalent carbohydrate binding proteins which have the ability to agglutinate red blood cells from a range of species. Lectins have been identified recently as insecticidal agents with activity against weevils, ECB and rootworm (Murdock et al., Phytochemistry, 29:85-89, 1990, Czapla & Lang, J. Econ. Entomol., 83:2480-2485, 1990). Lectin genes contemplated to be useful include, for example, barley and wheat germ agglutinin (WGA) and rice lectins (Gatehouse et al., J. Sci. Food. Agric., 35:373-380, 1984).

Genes controlling the production of large or small polypeptides active against insects when introduced into the insect pests, such as, e.g., lytic peptides, peptide hormones and toxins and venoms, form another aspect of the invention. For example, it is contemplated that the expression of juvenile hormone esterase, directed towards specific insect pests, also may result in insecticidal activity, or perhaps cause cessation of metamorphosis (Hammock et al., Nature, 344:458-461, 1990).

Genes that encode enzymes that affect the integrity of the insect cuticle form yet another aspect of the invention. Such genes include those encoding, e.g., chitinase, proteases, lipases and also genes for the production of nikkomycin, a compound that inhibits chitin synthesis, the introduction of any of which is contemplated to produce insect resistant plants. Genes that code for activities that affect insect molting, such as those affecting the production of ecdysteroid UDP glucosyl transferase, also fall within the scope of the useful exogenous nucleic acids of the present invention.

Genes that code for enzymes that facilitate the production of compounds that reduce the nutritional quality of the host sugar cane plant to insect pests also are encompassed by the present invention. It may be possible, for instance, to confer insecticidal activity on a sugar cane plant by altering its sterol composition. Sterols are obtained by insects from their diet and are used for hormone synthesis and membrane stability. Therefore alterations in plant sterol composition by expression of novel genes, e.g., those that directly promote the production of undesirable sterols or those that convert desirable sterols into undesirable forms, could have a negative effect on insect growth and/or development and hence endow the plant with insecticidal activity. Lipoxygenases are naturally occurring plant enzymes that have been shown to exhibit anti nutritional effects on insects and to reduce the nutritional quality of their diet. Therefore, further embodiments of the invention concern modified plants with enhanced lipoxygenase activity which may be resistant to insect feeding.

Tripsacum dactyloides is a species of grass that is resistant to certain insects, including root worm. It is anticipated that genes encoding proteins that are toxic to insects or are involved in the biosynthesis of compounds toxic to insects will be isolated from Tripsacum and that these novel genes will be useful in conferring resistance to insects. It is known that the basis of insect resistance in Tripsacum is genetic, because said resistance has been transferred to Zea mays via sexual crosses (Branson and Guss, Proceedings North Central Branch Entomological Society of America, 27:91-95, 1972). It is further anticipated that other cereal, monocot or dicot plant species may have genes encoding proteins that are toxic to insects which would be useful for producing insect resistant sugar cane plants.

Further genes encoding proteins characterized as having potential insecticidal activity also may be used as exogenous nucleic acids in accordance herewith. Such genes include, for example, the cowpea trypsin inhibitor (CpTI; Hilder et al., Nature, 330:160-163, 1987) which may be used as a rootworm deterrent; genes encoding avermectin (Avermectin and Abamectin., Campbell, W. C., Ed., 1989; Ikeda et al., J. Bacteriol., 169:5615-5621, 1987) which may prove particularly useful as a corn rootworm deterrent; ribosome inactivating protein genes; and even genes that regulate plant structures. sugar cane plants comprising a sugar cane mini-chromosome comprising anti insect antibody genes and genes that code for enzymes that can convert a non toxic insecticide (pro insecticide) applied to the outside of the plant into an insecticide inside the plant also are contemplated.

Polypeptides that may improve sugar cane tolerance to the effects of plant pests or pathogens include proteases, polypeptides involved in anthocyanin biosynthesis, polypeptides involved in cell wall metabolism, including cellulases, glucosidases, pectin methylesterase, pectinase, polygalacturonase, chitinase, chitosanase, and cellulose synthase, and polypeptides involved in biosynthesis of terpenoids or indole for production of bioactive metabolites to provide defense against herbivorous insects. It is also anticipated that combinations of different insect resistance genes on the same sugar cane mini-chromosome will be particularly useful.

Vegetative Insecticidal Proteins (VIP) are another class of proteins originally found to be produced in the vegetative growth phase of the bacterium, Bacillus cereus, but do have a spectrum of insect lethality similar to the insecticidal genes found in strains of Bacillus thuringiensis. Both the vip1a and vip3A genes have been isolated and have demonstrated insect toxicity. It is anticipated that such genes may be used in modified plants to confer insect resistance (“Plants, Genes, and Crop Biotechnology” by Maarten J. Chrispeels and David E. Sadava (2003) Jones and Bartlett Press).

(iii) Environment or Stress Resistance

Improvement of a sugar cane plant's ability to tolerate various environmental stresses such as, but not limited to, drought, excess moisture, chilling, freezing, high temperature, salt, and oxidative stress, also can be effected through expression of novel genes. It is proposed that benefits may be realized in terms of increased resistance to freezing temperatures through the introduction of an “antifreeze” protein such as that of the Winter Flounder (Cutler et al., J. Plant Physiol., 135:351-354, 1989) or synthetic gene derivatives thereof. Improved chilling tolerance also may be conferred through increased expression of glycerol 3 phosphate acetyltransferase in chloroplasts (Wolter et al., The EMBO J., 4685-4692, 1992). Resistance to oxidative stress (often exacerbated by conditions such as chilling temperatures in combination with high light intensities) can be conferred by expression of superoxide dismutase, and may be improved by glutathione reductase (Bowler et al., Ann Rev. Plant Physiol., 43:83-116, 1992). Such strategies may allow for tolerance to freezing in newly emerged fields as well as extending later maturity higher yielding varieties to earlier relative maturity zones.

It is contemplated that the expression of novel genes that favorably affect sugar cane plant water content, total water potential, osmotic potential, or turgor will enhance the ability of the plant to tolerate drought. As used herein, the terms “drought resistance.” and “drought tolerance” are used to refer to a sugar cane plant's increased resistance or tolerance to stress induced by a reduction in water availability, as compared to normal circumstances, and the ability of the plant to function and survive in lower water environments. In this aspect of the invention it is proposed, for example, that the expression of genes encoding for the biosynthesis of osmotically active solutes, such as polyol compounds, may impart protection against drought. Within this class are genes encoding for mannitol L phosphate dehydrogenase and trehalose 6 phosphate synthase (Kaaren et al., J. Bacteriology, 174:889-898, 1992). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski et al., Science, 259:508-510, 1993, Tarczynski et al Proc. Natl. Acad. Sci. USA, 89:1-5, 1993).

Similarly, the efficacy of other metabolites in protecting either enzyme function (e.g., alanopine or propionic acid) or membrane integrity (e.g., alanopine) has been documented (Loomis et al., J. Expt. Zoology, 252:9-15, 1989), and therefore expression of genes encoding for the biosynthesis of these compounds might confer drought resistance in a manner similar to or complimentary to mannitol. Other examples of naturally occurring metabolites that are osmotically active and/or provide some direct protective effect during drought and/or desiccation include fructose, erythritol (Coxson et al., Biotropica, 24:121-133, 1992), sorbitol, dulcitol (Karsten et al., Botanica Marina, 35:11-19, 1992), glucosylglycerol (Reed et al., J. Gen. Microbiology, 130:1-4, 1984; Erdmann et al., J. Gen. Microbiology, 138:363-368, 1992), sucrose, stachyose (Koster and Leopold, Plant Physiol., 88:829-832, 1988; Blackman et al., Plant Physiol., 100:225-230, 1992), raffinose (Bernal Lugo and Leopold, Plant Physiol., 98:1207-1210, 1992), proline (Rensburg et al., J. Plant Physiol., 141:188-194, 1993), glycine betaine, ononitol and pinitol (Vernon and Bohnert, The EMBO J., 11:2077-2085, 1992). Continued growth and increased reproductive fitness during times of stress may be augmented by introduction and expression of genes such as those controlling the osmotically active compounds discussed above and other such compounds. Genes which promote the synthesis of an osmotically active polyol compound include genes which encode the enzymes mannitol 1 phosphate dehydrogenase, trehalose 6 phosphate synthase and myoinositol 0 methyltransferase.

It is contemplated that the expression of specific proteins also may increase drought tolerance in sugar cane. Three classes of Late Embryogenic Abundant (LEA) Proteins have been assigned based on structural similarities (see Dure et al., Plant Molecular Biology, 12:475-486, 1989). All three classes of LEAs have been demonstrated in maturing (e.g. desiccating) seeds. Within these 3 types of LEA proteins, the Type II (dehydrin type) have generally been implicated in drought and/or desiccation tolerance in vegetative plant parts (e.g. Mundy and Chua, The EMBO J., 7:2279-2286, 1988; Piatkowski et al., Plant Physiol., 94:1682-1688, 1990; Yamaguchi Shinozaki et al., Plant Cell Physiol., 33:217-224, 1992). Expression of a Type III LEA (HVA 1) in tobacco was found to influence plant height, maturity and drought tolerance (Fitzpatrick, Gen. Engineering News, 22:7, 1993). In rice, expression of the HVA 1 gene influenced tolerance to water deficit and salinity (Xu et al., Plant Physiol., 110:249-257, 1996). Expression of structural genes from any of the three LEA groups may therefore confer drought tolerance. Other types of proteins induced during water stress include thiol proteases, aldolases or transmembrane transporters (Guerrero et al., Plant Molecular Biology, 15:11-26, 1990), which may confer various protective and/or repair type functions during drought stress. It also is contemplated that genes that effect lipid biosynthesis and hence membrane composition might also be useful in conferring drought resistance in sugar cane.

Many of these genes for improving drought resistance have complementary modes of action. Thus, it is envisaged that combinations of these genes might have additive and/or synergistic effects in improving drought resistance in sugar cane. Many of these genes also improve freezing tolerance (or resistance); the physical stresses incurred during freezing and drought are similar in nature and may be mitigated in similar fashion. Benefits may be conferred via constitutive expression of these genes; alternatively, one means of expressing these novel genes may be through the use of a turgor induced promoter (such as the promoters for the turgor induced genes described in Guerrero et al., Plant Molecular Biology, 15:11-26, 1990 and Shagan et al., Plant Physiol., 101:1397-1398, 1993). Spatial and temporal expression patterns of these genes may enable plants to better withstand stress.

It is proposed that expression of genes that are involved with specific morphological traits that allow for increased water extractions from drying soil would be of benefit. For example, introduction and expression of genes that alter root characteristics may enhance water uptake. It also is contemplated that expression of genes that enhance reproductive fitness during times of stress would be of significant value. For example, expression of genes that improve the synchrony of pollen shed and receptiveness of the female flower parts, e.g., silks, would be of benefit. In addition it is proposed that expression of genes that minimize kernel abortion during times of stress would increase the amount of grain to be harvested and hence be of value.

Given the overall role of water in determining yield, it is contemplated that enabling sugar cane to utilize water more efficiently, through the introduction and expression of novel genes, will improve overall performance even when soil water availability is not limiting. By introducing genes that improve the ability of sugar cane plants to maximize water usage across a full range of stresses relating to water availability, yield stability or consistency of yield performance may be realized.

Polypeptides that may improve stress tolerance in sugar cane under a variety of stress conditions include polypeptides involved in gene regulation, such as serine/threonine-protein kinases, MAP kinases, MAP kinase kinases, and MAP kinase kinase kinases; polypeptides that act as receptors for signal transduction and regulation, such as receptor protein kinases; intracellular signaling proteins, such as protein phosphatases, GTP binding proteins, and phospholipid signaling proteins; polypeptides involved in arginine biosynthesis; polypeptides involved in ATP metabolism, including for example ATPase, adenylate transporters, and polypeptides involved in ATP synthesis and transport; polypeptides involved in glycine betaine, jasmonic acid, flavonoid or steroid biosynthesis; and hemoglobin. Enhanced or reduced activity of such polypeptides in sugar cane plants comprising a sugar cane mini-chromosome will provide changes in the ability of the plants to respond to a variety of environmental stresses, such as chemical stress, drought stress and pest stress.

Other polypeptides that may improve sugar cane tolerance to cold or freezing temperatures include polypeptides involved in biosynthesis of trehalose or raffinose, polypeptides encoded by cold induced genes, fatty acyl desaturases and other polypeptides involved in glycerolipid or membrane lipid biosynthesis, which find use in modification of membrane fatty acid composition, alternative oxidase, calcium-dependent protein kinases, LEA proteins or uncoupling protein.

Other polypeptides that may improve sugar cane tolerance to heat include polypeptides involved in biosynthesis of trehalose, polypeptides involved in glycerolipid biosynthesis or membrane lipid metabolism (for altering membrane fatty acid composition), heat shock proteins or mitochondrial NDK.

Other polypeptides that may improve sugar cane tolerance to extreme osmotic conditions include polypeptides involved in proline biosynthesis.

Other polypeptides that may improve sugar cane tolerance to drought conditions include aquaporins, polypeptides involved in biosynthesis of trehalose or wax, LEA proteins or invertase.

(iv) Disease Resistance

It is proposed that increased resistance (or tolerance) to diseases may be realized through introduction of genes into sugar cane. It is possible to produce resistance to diseases caused by viruses, viroids, bacteria, fungi and nematodes. It also is contemplated that control of mycotoxin producing organisms may be realized through expression of introduced genes.

Resistance can be affected through suppression of endogenous factors that encourage disease-causing interactions, expression of exogenous factors that are toxic to or otherwise provide protection from pathogens, or expression of factors that enhance sugar cane's own defense responses.

Resistance to viruses may be produced through expression of novel genes in sugar cane. For example, it has been demonstrated that expression of a viral coat protein in a modified plant can impart resistance to infection of the plant by that virus and perhaps other closely related viruses (Cuozzo et al., Bio/Technology, 6:549-553, 1988, Hemenway et al., The EMBO J., 7:1273-1280, 1988, Abel et al., Science, 232:738-743, 1986). It is contemplated that expression of antisense genes targeted at essential viral functions may also impart resistance to viruses. For example, an antisense gene targeted at the gene responsible for replication of viral nucleic acid may inhibit replication and lead to resistance to the virus. It is believed that interference with other viral functions through the use of antisense genes also may increase resistance to viruses. Further, it is proposed that it may be possible to achieve resistance to viruses through other approaches, including, but not limited to the use of satellite viruses.

It is proposed that increased resistance to diseases caused by bacteria and fungi sugar cane may be realized through introduction of novel genes. It is contemplated that genes encoding so called “peptide antibiotics,” pathogenesis related (PR) proteins, toxin resistance, or proteins affecting host pathogen interactions such as morphological characteristics will be useful. Peptide antibiotics are polypeptide sequences which are inhibitory to growth of bacteria and other microorganisms. For example, the classes of peptides referred to as cecropins and magainins inhibit growth of many species of bacteria and fungi. It is proposed that expression of PR proteins in sugar cane may be useful in conferring resistance to bacterial disease. These genes are induced following pathogen attack on a host plant and have been divided into at least five classes of proteins (Bol, Linthorst, and Cornelissen, 1990). Included amongst the PR proteins are beta 1, 3 glucanases, chitinases, and osmotin and other proteins that are believed to function in plant resistance to disease organisms. Other genes have been identified that have antifungal properties, e.g., UDA (stinging nettle lectin), or hevein (Broakaert et al., 1989; Barkai Golan et al., 1978). It is known that certain plant diseases are caused by the production of phytotoxins. It is proposed that resistance to these diseases would be achieved through expression of a novel gene that encodes an enzyme capable of degrading or otherwise inactivating the phytotoxin. It also is contemplated that expression of novel genes that alter the interactions between the sugar cane host and pathogen may be useful in reducing the ability of the disease organism to invade the tissues of the host plant, e.g., an increase in the waxiness of the leaf cuticle or other morphological characteristics.

Polypeptides useful for imparting improved disease responses to sugar cane include polypeptides encoded by cercosporin induced genes, antifungal proteins and proteins encoded by R-genes or SAR genes.

Agronomically important diseases in sugar cane include but are not limited to: pineapple disease of sugar cane, pokkah boeng disease of sugar cane, sugar cane eye spot disease, sugar cane leaf scald disease, sugar cane mosaic virus disease, sugar cane ratoon stunting disease, sugar cane red rot Disease, sugar cane rust Disease, sugar cane smut disease, Metarhizium anisopliae, Ustilago scitaminea, Colletotrichum falcatum, Fusarium moniliformae, Cephalosporium sacchari, Certocystis paradoxa, Cercospora, Helminthosporium and Leptosphaeria, Puccinia. graminicolum, Puccinia aphanidermatum and Puccinia catenulatum, Xanthomonas albilineans, Leifsonia xyli, sugar cane mosaic virus (SCMV) (Potyvirdae), sugar cane bacilliform virus (SCBV) (Pararetroviridae), sugar cane yellow leaf syndrome (YLS), and sugar cane yellow leaf virus (ScYLV).

### Enhanced Biofuel Conversion

Biofuels may be produced from the conversion of sugar cane biomass into liquid or gaseous fuels by converting the biomass into sugars, or by direct extraction of sugars, that can be fermented or chemically converted to form a biofuel. Biofuels can also be generated by extracting oils from the biomass. Exemplary biofuels are ethanol, propanol, butanol, methanol, methane, 2,5-dimethylfurgan, dimethyl ether, biodiesel (short chain acid alkyl esters), biogasoline, parrafins (alkanes), other hydrocarbons or co-products of hydrogen.

The invention provides for sugar cane mini-chromosomes expressing at least one gene that enhances or increases sugar production or extractability, enhances or increases biomass, enhances the conversion of biomass to sugars or enhances sugar fermentation to biofuels. It may further be considered that a modified sugar cane plant prepared in accordance with the invention may be used as biomass for the production of biofuels or the plant may facilitate conversion of biomass to sugars or facilitate fermentation of sugars to biofuels.

Enzymes that may be useful for biofuel production include those that break down glucans. In some embodiments, the enzymes are selected from the group consisting of: endo-β(1,4)-glucanase, cellobiohydrolase, β-glucosidase, α/β-glucosidase, mixed-linked glucanase, endo-β(1,3)-glucanase, exo-β(1,3)-glucanase and β-(1,6)-glucanase. In other embodiments the enzymes break down xyloglucans, xylans, mannans or lignins.

The enzyme genes may be controlled by inducible promoters that may be inactive until a desired time, such as at harvest or when the plant is added to the biofuels process (e.g. inactive at physiological conditions, then activated by heat or pH), or sequestered by subcellular localization. The enzymes may also be controlled by a tissue-specific promoter which may be active only in specific tissues (e.g. seeds or leaves).

### Non-Protein-Expressing Exogenous Nucleic Acids

Sugar cane plants with decreased expression of a gene of interest can also be achieved, for example, by expression of antisense nucleic acids, dsRNA or RNAi, catalytic RNA such as ribozymes, sense expression constructs that exhibit cosuppression effects, aptamers or zinc finger proteins.

Antisense RNA reduces production of the polypeptide product of the target messenger RNA, for example by blocking translation through formation of RNA:RNA duplexes or by inducing degradation of the target mRNA. Antisense approaches are a way of preventing or reducing gene function by targeting the genetic material as disclosed in U.S. Pat. Nos. 4,801,540; 5,107,065; 5,759,829; 5,910,444; 6,184,439; and 6,198,026. In one approach, an antisense gene sequence is introduced that is transcribed into antisense RNA that is complementary to the target mRNA. For example, part or all of the normal gene sequences are placed under a promoter in inverted orientation so that the complementary strand is transcribed into a non-protein expressing antisense RNA. The promoter used for the antisense gene may influence the level, timing, tissue, specificity, or inducibility of the antisense inhibition.

Autonomous sugar cane mini-chromosomes may comprise exogenous DNA flanked by recombination sites, for example lox-P sites, that can be recognized by a recombinase, e.g. Cre, and removed from the sugar cane mini-chromosome. In cases where there is a homologous recombination site or sites in the host genomic DNA, the exogenous DNA excised from the sugar cane mini-chromosome may be integrated into the genome at one of the specific recombination sites and the DNA flanked by the recombination sites will become integrated into the host DNA. The use of a sugar cane mini-chromosome as a platform for DNA excision or for launching such DNA integration into the host genome may include in vivo induction of the expression of a recombinase encoded in the genomic DNA of a transgenic host, or in a sugar cane mini-chromosome.

RNAi gene suppression in plants by transcription of a dsRNA is described in U.S. Pat. No. 6,506,559, U.S. patent application Publication No. 2002/0168707, WO 98/53083, WO 99/53050 and WO 99/61631. The double-stranded RNA or RNAi constructs can trigger the sequence-specific degradation of the target messenger RNA. Suppression of a gene by RNAi can be achieved using a recombinant DNA construct having a promoter operably linked to a DNA element comprising a sense and anti-sense element of a segment of genomic DNA of the gene, e.g., a segment of at least about 23 nucleotides, optionally about 50 to 200 nucleotides where the sense and anti-sense DNA components can be directly linked or joined by an intron or artificial DNA segment that can form a loop when the transcribed RNA hybridizes to form a hairpin structure.

Catalytic RNA molecules or ribozymes can also be used to inhibit expression of the target gene or genes or facilitate molecular reactions. Ribozymes are targeted to a given sequence by hybridization of sequences within the ribozyme to the target mRNA. Two stretches of homology are required for this targeting, and these stretches of homologous sequences flank the catalytic ribozyme structure. It is possible to design ribozymes that specifically pair with virtually any target mRNA and cleave the target mRNA at a specific location, thereby inactivating it. A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include Tobacco Ringspot Virus (Prody et al., Science, 231:1577-1580, 1986), Avocado Sunblotch Viroid (Palukaitis et al., Virology, 99:145-151, 1979; Symons, Nucl. Acids Res., 9:6527-6537, 1981), and Lucerne Transient Streak Virus (Forster and Symons, Cell, 49:211-220, 1987), and the satellite RNAs from velvet tobacco mottle virus, Solanum nodiflorum mottle virus and subterranean clover mottle virus. The design and use of target RNA-specific ribozymes is described in Haseloff, et al., Nature 334:585-591 (1988). Several different ribozyme motifs have been described with RNA cleavage activity (Symons, Annu. Rev. Biochem., 61:641-671, 1992). Other suitable ribozymes include sequences from RNase P with RNA cleavage activity (Yuan et al., Proc. Natl. Acad. Sci. USA, 89:8006-8010, 1992; Yuan and Altman, Science, 263:1269-1273, 1994; U.S. Pat. Nos. 5,168,053 and 5,624,824), hairpin ribozyme structures (Berzal-Herranz et al., Genes and Devel., 6:129-134, 1992; Chowrira et al., J. Biol. Chem., 269:25856-25864, 1994) and Hepatitis Delta virus based ribozymes (U.S. Pat. No. 5,625,047). The general design and optimization of ribozyme directed RNA cleavage activity has been discussed in detail (Haseloff and Gerlach, 1988, Nature. 1988 Aug. 18; 334(6183):585-91, Chowrira et al., J. Biol. Chem., 269:25856-25864, 1994).

Another method of reducing protein expression utilizes the phenomenon of cosuppression or gene silencing (for example, U.S. Pat. Nos. 6,063,947; 5,686,649; or U.S. Pat. No. 5,283,184). Cosuppression of an endogenous gene using a full-length cDNA sequence as well as a partial cDNA sequence are known (for example, Napoli et al., Plant Cell 2:279-289 [1990]; van der Krol et al., Plant Cell 2:291-299 [1990]; Smith et al., Mol. Gen. Genetics 224:477-481 [1990]). The phenomenon of cosuppression has also been used to inhibit plant target genes in a tissue-specific manner.

In some embodiments, nucleic acids from one species of plant are expressed in another species of plant to effect cosuppression of a homologous gene. The introduced sequence generally will be substantially identical to the endogenous sequence intended to be repressed, for example, about 65%, 80%, 85%, 90%, 95% or even 98% or greater identical. Higher identity may result in a more effective repression of expression of the endogenous sequence. A higher identity in a shorter than full length sequence compensates for a longer, less identical sequence. Furthermore, the introduced sequence need not have the same intron or exon pattern, and identity of non-coding segments will be equally effective. Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence.

Yet another method of reducing protein activity is by expressing nucleic acid ligands, so-called aptamers, which specifically bind to the protein. Aptamers may be obtained by the SELEX (Systematic Evolution of Ligands by Exponential Enrichment) method. See U.S. Pat. No. 5,270,163. In the SELEX method, a candidate mixture of single stranded nucleic acids having regions of randomized sequence is contacted with the protein and those nucleic acids having an increased affinity to the target are selected and amplified. After several iterations a nucleic acid with optimal affinity to the polypeptide is obtained and is used for expression in modified plants.

A zinc finger protein that binds a polypeptide-encoding sequence or its regulatory region is also used to alter expression of the nucleotide sequence. Transcription of the nucleotide sequence may be reduced or increased. Zinc finger proteins are, for example, described in Beerli et al. (1998) PNAS 95:14628-14633., or in WO 95/19431, WO 98/54311, or WO 96/06166.

Other examples of non-protein expressing sequences specifically envisioned for use with the invention include tRNA sequences, for example, to alter codon usage, and rRNA variants, for example, which may confer resistance to various agents such as antibiotics.

It is contemplated that unexpressed DNA sequences, including novel synthetic sequences, could be introduced into sugar cane cells as proprietary “labels” of those cells and plants and seeds thereof. It would not be necessary for a label DNA element to disrupt the function of a gene endogenous to the host organism, as the sole function of this DNA would be to identify the origin of the organism. For example, one could introduce a unique DNA sequence into a sugar cane plant and this DNA element would identify all cells, plants, and progeny of these cells as having arisen from that labeled source. It is proposed that inclusion of label DNAs would enable one to distinguish proprietary germplasm or germplasm derived from such, from unlabelled germplasm.

### Exemplary Plant Promoters, Regulatory Sequences and Targeting Sequences

A number of elements may optionally be included in the polynucleotides, nucleic acids, centromeres and sugar cane mini-chromosomes of the invention.

The promoter in the sugar cane mini-chromosome of the present invention can be derived from plant or non-plant species or can be partially or wholly synthetic. In one embodiment, the nucleotide sequence of the promoter is derived from non-plant species for the expression of genes in plant cells. Exemplary classes of plant promoters are described below.

Constitutive Expression promoters: Exemplary constitutive expression promoters include the ubiquitin promoter (e.g., sunflower—Binet et al. Plant Science 79: 87-94 (1991); maize—Christensen et al. Plant Molec. Biol. 12: 619-632 (1989); and Arabidopsis—Callis et al., J. Biol. Chem. 265: 12486-12493 (1990) and Norris et al., Plant Mol. Biol. 21: 895-906 (1993)); the CaMV ³⁵S promoter (U.S. Pat. Nos. 5,858,742 and 5,322,938); or the actin promoter (e.g., rice—U.S. Pat. No. 5,641,876; McElroy et al. Plant Cell 2: 163-171 (1990),

McElroy et al. Mol. Gen. Genet. 231: 150-160 (1991), and Chibbar et al. Plant Cell Rep. 12: 506-509 (1993)). Exemplary promoters for use in sugar cane include the maize polyubiquitin 1 (Mubi-1) and the sugar cane polyubiquitin 9 (SCubi9) promoters (Wang M L, Goldstein C, Su W, Moore P H, Albert H H. Production of biologically active GM-CSF in sugar cane: a secure biofactory. Transgenic Res. 2005, 14:167-78); and the sugar cane polyubiquitin 4 (ubi4) promoter (Wei H, Wang M L, Moore P H, Albert H H. Comparative expression analysis of two sugar cane polyubiquitin promoters and flanking sequences in transgenic plants. J Plant Physiol. 2003, 160:1241-51). Inducible Expression promoters: Exemplary inducible expression promoters include the chemically regulatable tobacco PR-1 promoter (e.g., tobacco—U.S. Pat. No. 5,614,395; Arabidopsis—Lebel et al., Plant J. 16: 223-233 (1998); maize—U.S. Pat. No. 6,429,362). Various chemical regulators may be employed to induce expression, including the benzothiadiazole, isonicotinic acid, and salicylic acid compounds disclosed in U.S. Pat. Nos. 5,523,311 and 5,614,395. Other promoters inducible by certain alcohols or ketones, such as ethanol, include, for example, the alcA gene promoter from Aspergillus nidulans (Caddick et al. (1998) Nat. Biotechnol 16:177-180). A glucocorticoid-mediated induction system is described in Aoyama and Chua (1997) The Plant Journal 11: 605-612 wherein gene expression is induced by application of a glucocorticoid, for example a dexamethasone. Another class of useful promoters are water-deficit-inducible promoters, e.g. promoters which are derived from the 5′ regulatory region of genes identified as a heat shock protein 17.5 gene (HSP 17.5), an HVA22 gene (HVA22), and a cinnamic acid 4-hydroxylase (CA4H) gene of Zea mays. Another water-deficit-inducible promoter is derived from the rab-17 promoter as disclosed by Vilardell et al., Plant Molecular Biology, 17(5):985-993, 1990. See also U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, and U.S. Pat. No. 6,175,060 which discloses phosphorus deficiency inducible promoters.

As another example, numerous wound-inducible promoters have been described (e.g. Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), Warner et al. Plant J. 3: 191-201 (1993)). Logemann et al., describe 5′ upstream sequences of the potato wunl gene. Xu et al. show that a wound-inducible promoter from the dicotyledon potato (pin2) is active in the monocotyledon rice. Rohrmeier & Lehle describe maize Wipl cDNA which is wound induced and which can be used to isolate the cognate promoter. Firek et al. and Warner et al. have described a wound-induced gene from the monocotyledon Asparagus officinalis, which is expressed at local wound and pathogen invasion sites.

Tissue-Specific and Tissue-Preferred Promoters: Exemplary promoters that express genes only or preferentially in certain sugar cane tissues are useful according to the present invention. For example root specific or preferred expression may be attained using the promoter of the maize metallothionein-like (MTL) gene described by de Framond (FEBS 290: 103-106 (1991)) and also in U.S. Pat. No. 5,466,785. U.S. Pat. No. 5,837,848 discloses a root specific promoter. Another exemplary promoter confers pith-preferred expression (see Int'l. Pub. No. WO 93/07278, which describes the maize trpA gene and promoter that is preferentially expressed in pith cells). Leaf-specific or—preferred expression may be attained, for example, by using the promoter for a maize gene encoding phosphoenol carboxylase (PEPC) (see Hudspeth & Grula, Plant Molec Biol 12: 579-589 (1989)). Pollen-specific or preferred expression may be conferred by the promoter for the maize calcium-dependent protein kinase (CDPK) gene which is expressed in pollen cells (WO 93/07278). U.S. Pat. Appl. Pub. No. 20040016025 describes tissue-specific promoters. Pollen-specific or preferred expression may be conferred by the tomato LAT52 pollen-specific promoter (Bate et. al., Plant Mol. Biol. 1998 July; 37(5):859-69).

See also U.S. Pat. No. 6,437,217 which discloses a root-specific maize RS81 promoter, U.S. Pat. No. 6,426,446 which discloses a root specific maize RS324 promoter, U.S. Pat. No. 6,232,526 which discloses a constitutive maize A3 promoter, U.S. Pat. No. 6,177,611 which discloses constitutive maize promoters, U.S. Pat. No. 6,433,252 which discloses a maize L3 oleosin promoter that are aleurone and seed coat-specific promoters, U.S. Pat. No. 6,429,357 which discloses a constitutive rice actin 2 promoter and intron, U.S. patent application Pub. No. 20040216189 which discloses an inducible constitutive leaf specific maize chloroplast aldolase promoter.

In one embodiment, a non-plant promoter, which can be constitutive or inducible, is used. Such promoters can, for example, be derived from insect, e.g., Drosophila melanogaster or yeast, e.g., Saccharomyces cerevisiae. Table 1 lists nonlimiting examples of promoters from Drosophila melanogaster and Saccharomyces cerevisiae that can be used to derive the examples of non-plant promoters in the present invention. Promoters derived from any animal, protist, or fungi are also contemplated. The promoter sequences shown in Table 1 or fragments, mutants, hybrid or tandem promoters thereof, are examples of promoter sequences derived from Drosophila melanogaster or Saccharomyces cerevisiae. These non-plant promoters can be operably linked to nucleic acid sequences encoding polypeptides or non-protein-expressing sequences including, but not limited to, antisense RNA and ribozymes, to form nucleic acid constructs, vectors, and host cells (prokaryotic or eukaryotic), comprising the promoters.

TABLE 1
Drosophila melanogaster Promoters (Information obtained from the Flybase Web
Site at http://flybase.bio.indiana.edu/which is a database of the Drosophila Genome)

| | | Standard promoter | | |
|---|---|---|---|---|
| SSym | Flybase ID | gene name | Gene Product | Chromosome |
| Pgd | FBgn0004654 | Phosphogluconate | 6-phosphogluconate | X |
| | | dehydrogenase | dehydrogenase | |
| Grim | FBgn0015946 | grim | grim-P138 | 3 |
| Uro | FBgn0003961 | Urate oxidase | Uro-P1 | 2 |
| Sna | FBgn0003448 | Snail | sna-P1 | 2 |
| Rh3 | FBgn0003249 | Rhodopsin 3 | Rh3 | 3 |
| Lsp-1 γ | FBgn0002564 | Larval serum | Lsp1γ-P1 | 3 |
| | | protein 1 γ | | |

Saccharomyces cerevisiae Promoters
(Information obtained from the Saccharomyces Genome Database Web site at
http://www.yeastgenome.org/SearchContents.shtml

| | Systematic | Standard promoter | | |
|---|---|---|---|---|
| SSymbol | Name | gene name | Gene Product | Chromosome |
| Tef-2 | YBR118W | TEF2 (Translation | Translation elongation | 2 |
| | | elongation factor | factor EF-1 alpha | |
| | | promoter) | | |
| Leu-1 | YGL009C | LEU1 (LEUcine | isopropylmalate | 7 |
| | | biosynthesis) | isomerase | |
| Met16 | YPR167C | METhionine | 3′phosphoadenylyl- | 16 |
| | | requiring | sulfate reductase | |
| Leu-2 | YCL018W | LEU2 (leucine | beta-IPM | 3 |
| | | biosynthesis) | (isopropylmalate) | |
| | | | dehydrogenase | |
| His-4 | YCL030C | HIS4 (HIStidine | histidinol | 3 |
| | | requiring) | dehydrogenase | |
| Met-2 | YNL277W | MET2 (methionine | L-homoserine-O- | 14 |
| | | requiring) | acetyltransferase | |
| | | Standard promoter | | |
| SSym | Flybase ID | gene name | Gene Product | Chromosome |
| Ste-3 | YKL178C | STE3 (alias DAF2 | a-factor receptor | 11 |
| | | Sterile) | | |
| Arg-1 | YOL058W | ARG1(alias ARG10 | arginosuccinate | 15 |
| | | ARGinine requiring) | synthetase | |
| Pgk-1 | YCR012W | PGK1 | phosphoglycerate kinase | 3 |
| | | (phosphoglycerate | | |
| | | kinase) | | |
| GPD-1 | YDL022W | GPD1 (alias | glycerol-3-phosphate | 4 |
| | | DAR1/HOR1/OSG1/ | dehydrogenase | |
| | | SR5: glycerol-3- | | |
| | | phosphate | | |
| | | dehydrogenase | | |
| | | activity | | |
| ADH1 | YOL086C | ADH1 (alias ADC1) | alcohol dehydrogenase | 15 |
| GPD-2 | YOL059W | GPD2 (alias GPD3: | glycerol-3-phosphate | 15 |
| | | glycerol-3- | dehydrogenase | |
| | | phosphate | | |
| | | dehydrogenase | | |
| | | activity | | |
| Arg-4 | YHR018C | ARGinine requiring | argininosuccinate lyase | 8 |
| Yat-1 | YAR035W | YAT-1(carnitine | carnitine | 1 |
| | | acetyltransferase) | acetyltransferase | |

In the sugar cane mini-chromosomes of the present invention, the promoter may be a variant of any of the foregoing promoters having, for example, a substitution, deletion, and/or insertion of one or more nucleotides in the nucleic acid sequences of Table 1.

Optionally a plant transcriptional terminator can be used in place of the plant-expressed gene native transcriptional terminator. Exemplary transcriptional terminators are those that are known to function in plants and include the CaMV ³⁵S terminator, the tml terminator, the nopaline synthase terminator and the pea rbcS E9 terminator. These can be used in both monocotyledons and dicotyledons.

Various intron sequences have been shown to enhance expression, particularly in monocotyledonous cells. For example, the introns of the maize Adh1 gene have been found to significantly enhance expression. Intron 1 was found to be particularly effective and enhanced expression in fusion constructs with the chloramphenicol acetyltransferase gene (Callis et al., Genes Develop. 1: 1183-1200 (1987)). The intron from the maize bronzel gene also enhances expression. Intron sequences have been routinely incorporated into plant transformation vectors, typically within the non-translated leader. U.S. Patent Application Publication 2002/0192813 discloses 5′, 3′ and intron elements useful in the design of effective plant expression vectors.

A number of non-translated leader sequences derived from viruses are also known to enhance expression, and these are particularly effective in dicotyledonous cells. Specifically, leader sequences from Tobacco Mosaic Virus (TMV, the “omega-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include, but are not limited to: picornavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Sarnow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie et al., Molecular Biology of RNA, pages 237-256 (1989); or Maize Chlorotic Mottle Virus leader (MCMV) (Lommel et al., Virology 81:382-385 (1991). See also, Della-Cioppa et al., Plant Physiology 84:965-968 (1987).

A minimal promoter may also be incorporated. Such a promoter has low background activity in plants when there is no transactivator present or when enhancer or response element binding sites are absent. One exemplary minimal promoter is the Bz1 minimal promoter, which is obtained from the bronze 1 gene of maize. Roth et al., Plant Cell 3: 317 (1991). A minimal promoter may also be created by use of a synthetic TATA element. The TATA element allows recognition of the promoter by RNA polymerase factors and confers a basal level of gene expression in the absence of activation (see generally, Mukumoto (1993) Plant Mol Biol 23: 995-1003; Green (2000) Trends Biochem Sci 25: 59-63).

Sequences controlling the targeting of gene products also may be included. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). These signal sequences can be fused to heterologous gene products to effect the import of heterologous products into the chloroplast (van den Broeck, et al. Nature 313: 358-363 (1985)). DNA encoding for appropriate signal sequences can be isolated from the 5′ end of the cDNAs encoding the RUBISCO protein, the CAB protein, the EPSP synthase enzyme, the GS2 protein or many other proteins which are known to be chloroplast localized. Other gene products are localized to other organelles such as the mitochondrion and the peroxisome (e.g. Unger et al. Plant Molec. Biol. 13: 411-418 (1989)). Examples of sequences that target to such organelles are the nuclear-encoded ATPases or specific aspartate amino transferase isoforms for mitochondria. Targeting cellular protein bodies has been described by Rogers et al. (Proc. Natl. Acad. Sci. USA 82: 6512-6516 (1985)). In addition, amino terminal and carboxy-terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)).

Another possible element which may be included is a matrix attachment region element (MAR), such as the chicken lysozyme A element, which can be positioned around an expressible gene of interest to effect an increase in overall expression of the gene and diminish position dependent effects upon incorporation into the plant genome (Stief et al., Nature, 341:343, 1989; Phi-Van et al., Mol. Cell. Biol., 10:2302-2307.1990).

### Constructing Mini-Chromosomes by Site-Specific Recombination

Sugar cane mini-chromosomes may be constructed using site-specific recombination sequences (for example those recognized by the bacteriophage P1 Cre recombinase, or the bacteriophage lambda integrase, or similar recombination enzymes). According to this embodiment, a compatible recombination site, or a pair of such sites, is present on both the sugar cane centromere containing DNA clones and the donor DNA clones. Incubation of the donor clone and the centromere clone in the presence of the recombinase enzyme causes strand exchange to occur between the recombination sites in the two plasmids; the resulting sugar cane mini-chromosomes contain sugar cane centromere sequences as well as mini-chromosome vector sequences. The DNA molecules formed in such recombination reactions are introduced into E. coli, other bacteria, yeast or sugar cane cells by common methods in the field including, but not limited to, heat shock, chemical transformation, electroporation, particle bombardment, whiskers, or other transformation methods followed by selection for marker genes including chemical, enzymatic, color, or other marker, allowing for the selection of transformants harboring mini-chromosomes.

### III. Transformation of Plant Cells and Plant Regeneration

Various methods may be used to deliver DNA into plant cells. These include biological methods, such as Agrobacterium, E. coli, and viruses, physical methods such as biolistic particle bombardment, nanocopoea device, the Stein beam gun, silicon carbide whiskers and microinjection, electrical methods such as electroporation, and chemical methods such as the use of poly-ethylene glycol and other compounds known to stimulate DNA uptake into cells. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). Transformation using silicon carbide whiskers, e.g. in maize, is described in Brisibe, J. Exp. Bot. 51(343):187-196 (2000) and Dunwell, Methods Mol. Biol. 111:375-82 (1999) and U.S. Pat. No. 5,464,765.

### Agrobacterium-Mediated Delivery

Agrobacterium-mediated transformation is one method for introducing a desired genetic element into a plant. Several Agrobacterium species mediate the transfer of a specific DNA known as “T-DNA” that can be genetically engineered to carry a desired piece of DNA into many plant species. Plasmids used for delivery contain the T-DNA flanking the nucleic acid to be inserted into the plant. The major events marking the process of T-DNA mediated pathogenesis are induction of virulence genes, processing and transfer of T-DNA.

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be modified by Agrobacterium and (b) that the modified cells or tissues can be induced to regenerate into whole plants. The third method is transformation of seeds, immature or mature embryos, apices or meristems with Agrobacterium. This method requires exposure of the meristematic cells of these tissues to Agrobacterium and micropropagation of the shoots or plant organs arising from these meristematic cells.

Those of skill in the art are familiar with procedures for growth and suitable culture conditions for Agrobacterium as well as subsequent inoculation procedures. Liquid, solid, or semi-solid culture media can be used. The density of the Agrobacterium culture used for inoculation and the ratio of Agrobacterium cells to explant can vary from one system to the next, as can media, growth procedures, timing and lighting conditions.

Transformation of dicotyledons using Agrobacterium has long been known in the art, and transformation of monocotyledons using Agrobacterium has also been described. See, WO 94/00977 and U.S. Pat. No. 5,591,616. See also, Negrotto et al., Plant Cell Reports 19: 798-803 (2000).

A number of wild-type and disarmed strains of Agrobacterium tumefaciens and Agrobacterium rhizogenes harboring Ti or Ri plasmids can be used for gene transfer into plants. In embodiments of the invention, the Agrobacterium hosts contain disarmed Ti and Ri plasmids that do not contain the oncogenes that cause tumorigenesis or rhizogenesis. Exemplary strains include Agrobacterium tumefaciens strain C58, a nopaline-type strain that is used to mediate the transfer of DNA into a plant cell, octopine-type strains such as LBA4404 or succinamopine-type strains, e.g., EHA101 or EHA105. The use of these strains for plant transformation has been reported and the methods are familiar to those of skill in the art.

U.S. Application No. 20040244075 published Dec. 2, 2004 describes improved methods of Agrobacterium-mediated transformation. The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., (1987) Plant Molec. Biol. 8:291-298). Alternatively, transformation efficiency may be enhanced by wounding the target tissue to be modified or transformed. Wounding of plant tissue may be achieved, for example, by punching, maceration, bombardment with microprojectiles, etc. (See e.g., Bidney et al., (1992) Plant Molec. Biol. 18:301-313).

In addition, another recent method described by Broothaerts, et. al. (Nature 433: 629-633, 2005) expands the bacterial genera that can be used to transfer genes into plants. This work involved the transfer of a disarmed Ti plasmid without T-DNA and another vector with T-DNA containing the marker enzyme beta-glucuronidase, into three different bacteria. Gene transfer was successful and this method significantly expands the tools available for gene delivery into plants.

### Microprojectile Bombardment Delivery

Another widely used technique to genetically transform plants involves the use of microprojectile bombardment. In this process; a nucleic acid containing the desired genetic elements to be introduced into the plant is deposited on or in small dense particles, e.g., tungsten, platinum, or 0.5 to 1.0 micron gold particles, which are then delivered at a high velocity into the plant tissue or plant cells using a specialized biolistics device. Many such devices have been designed and constructed; one in particular, the PDS 1000/He sold by BioRad, is the instrument most commonly used for biolistics of plant cells. The advantage of this method is that no specialized sequences need to be present on the nucleic acid molecule to be delivered into plant cells; delivery of any nucleic acid sequence is theoretically possible.

For the bombardment, cells in suspension are concentrated on filters, petri dishes or solid culture medium. Alternatively, immature embryos, seedling explants, or any plant tissue or target cells may be arranged on filters, petri dishes or solid culture medium. The cells to be bombarded are positioned at an appropriate distance below the microprojectile stopping plate.

Various biolistics protocols have been described that differ in the type of particle or the manner in which DNA is coated onto the particle. Any technique for coating microprojectiles that allows for delivery of transforming DNA to the target cells may be used. For example, particles may be prepared by functionalizing the surface of a gold particle by providing free amine groups. DNA, having a strong negative charge, will then bind to the functionalized particles.

Parameters such as the concentration of DNA used to coat microprojectiles may influence the recovery of transformants containing a single copy of the transgene. For example, a lower concentration of DNA may not necessarily change the efficiency of the transformation but may instead increase the proportion of single copy insertion events. In this regard, ranges of approximately 1 ng to approximately 10 μg (10,000 ng), approximately 5 ng to 8 μg or approximately 20 ng, 50 ng, 100 ng, 200 ng, 500 ng, 1 μg, 2 μg, 5 μg, or 7 μg of transforming DNA may be used per each 1.0-2.0 mg of starting gold particles (in the 0.5 to 1.0 micron range).

Other physical and biological parameters may be varied, such as manipulation of the DNA/microprojectile precipitate, factors that affect the flight and velocity of the projectiles, manipulation of the cells before and immediately after bombardment (including osmotic state, tissue hydration and the subculture stage or cell cycle of the recipient cells), the orientation of an immature embryo or other target tissue relative to the particle trajectory, and also the nature of the transforming DNA, such as, linearized DNA or intact supercoiled plasmids. One may also want to use agents to protect the DNA during delivery. One may particularly wish to adjust physical parameters such as DNA concentration, gap distance, flight distance, tissue distance, and helium pressure.

The particles delivered via biolistics can be “dry” or “wet.” In the “dry” method, the mini-chromosome DNA-coated particles such as gold are applied onto a macrocarrier (such as a metal plate, or a carrier sheet made of a fragile material such as mylar) and dried. The gas discharge then accelerates the macrocarrier into a stopping screen, which halts the macrocarrier but allows the particles to pass through; the particles then continue their trajectory until they impact the tissue being bombarded. For the “wet” method, the droplet containing the mini-chromosome DNA-coated particles is applied to the bottom part of a filter holder, which is attached to a base which is itself attached to a rupture disk holder used to hold the rupture disk to the helium egress tube for bombardment. The gas discharge directly displaces the DNA/gold droplet from the filter holder and accelerates the particles and their DNA cargo into the tissue being bombarded. The wet biolistics method has been described in detail elsewhere but has not previously been applied in the context of plants (Mialhe et al., Mol Mar Biol Biotechnol. 4(4):275-83, 1995). The concentrations of the various components for coating particles and the physical parameters for delivery can be optimized using procedures known in the art.

A variety of sugar cane cells/tissues are suitable for transformation, including immature embryos, scutellar tissue, suspension cell cultures, immature inflorescence, shoot meristem, epithelial peels, nodal explants, callus tissue, hypocotyl tissue, cotyledons, roots, leaves, meristem cells, and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspore-derived embryos, roots, hypocotyls, cotyledons and the like. Those cells which are capable of proliferating as callus also are recipient cells for genetic transformation.

Any suitable plant culture medium can be used. Examples of suitable media would include but are not limited to MS-based media (Murashige and Skoog, Physiol. Plant, 15:473-497, 1962) or N6-based media (Chu et al., Scientia Sinica 18:659, 1975) supplemented with additional plant growth regulators including but not limited to auxins such as picloram (4-amino-3,5,6-trichloropicolinic acid), 2,4-D (2,4-dichlorophenoxyacetic acid), naphalene-acetic acid (NAA) and dicamba (3,6-dichloroanisic acid), cytokinins such as BAP (6-benzylaminopurine) and kinetin, and gibberellins. Other media additives can include but are not limited to amino acids, macroelements, iron, microelements, vitamins and organics, carbohydrates, undefined media components such as casein hydrolysates, an appropriate gelling agent such as a form of agar, a low melting point agarose or Gelrite if desired. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Examples of such media would include but are not limited to Murashige and Skoog (Murashige and Skoog, Physiol. Plant, 15:473-497, 1962), N6 (Chu et al., Scientia Sinica 18:659, 1975), Linsmaier and Skoog (Linsmaier and Skoog, Physio. Plant., 18:100, 1965), Uchimiya and Murashige (Uchimiya and Murashige, Plant Physiol. 15:473, 1962), Gamborg's B5 media (Gamborg et al., Exp. Cell Res., 50:151, 1968), D medium (Duncan et al., Planta, 165:322-332, 1985), Mc-Cown's

Woody plant media (McCown and Lloyd, HortScience 6:453, 1981), Nitsch and Nitsch (Nitsch and Nitsch, Science 163:85-87, 1969), and Schenk and Hildebrandt (Schenk and Hildebrandt, Can. J. Bot. 50:199-204, 1972) or derivations of these media supplemented accordingly. Those of skill in the art are aware that media and media supplements such as nutrients and growth regulators for use in transformation and regeneration and other culture conditions such as light intensity during incubation, pH, and incubation temperatures can be varied.

Those of skill in the art are aware of the numerous modifications in selective regimes, media, and growth conditions that can be varied depending on the plant system and the selective agent. Typical selective agents include but are not limited to antibiotics such as geneticin (G418), kanamycin, paromomycin or other chemicals such as glyphosate or other herbicides. Consequently, such media and culture conditions disclosed in the present invention can be modified or substituted with nutritionally equivalent components, or similar processes for selection and recovery of transgenic events, and still fall within the scope of the present invention.

Sugar Cane Mini-Chromosome Delivery without Selection

The following is an exemplary method for sugar cane mini-chromosome delivery without selection: the sugar cane mini-chromosome is delivered to sugar cane plant cells or tissues, e.g., plant cells in suspension to obtain stably modified callus clones for inheritance assay. Suspension cells are maintained in a growth media, for example Murashige and Skoog (MS) liquid medium containing an auxin such as 2,4-dichlorophenoxyacetic acid (2,4-D).

Cells are bombarded using a particle bombardment process, such as the helium-driven PDS-1000/He system, and propagated in the same liquid medium to permit the growth of modified and non-modified cells. Portions of each bombardment are monitored for formation of fluorescent clusters, which are isolated by micromanipulation and cultured on solid medium. Clones modified with the mini-chromosome are expanded and homogenous clones are used in inheritance assays, or assays measuring mini-chromosome structure or autonomy.

Sugar Cane Mini-Chromosome Transformation with Selectable Marker Gene

The following is an exemplary method for sugar cane mini-chromosome transformation with a selectable marker gene: isolation of sugar cane mini-chromosome-modified cells in bombarded calluses or explants can be facilitated by the use of a selectable marker gene. The bombarded tissues are transferred to a medium containing an appropriate selective agent for a particular selectable marker gene. Such a transfer usually occurs between about 0 and about 7 days after bombardment. The transfer could also take place any number of days after bombardment. The amount of selective agent and timing of incorporation of such an agent in selection medium can be optimized by using procedures known in the art. Selection inhibits the growth of non-modified cells, thus providing an advantage to the growth of modified cells, which can be further monitored by tracking the presence of a fluorescent marker gene or by the appearance of modified explants (modified cells or explants may be green under light in selection medium, while surrounding non-modified cells are weakly pigmented). In plants that develop through shoot organogenesis, the modified cells can form shoots directly, or alternatively, can be isolated and expanded for regeneration of multiple shoots transgenic for the sugar cane mini-chromosome. In plants that develop through embryogenesis, additional culturing steps may be necessary to induce the modified cells to form an embryo and to regenerate in the appropriate media. Sugar cane is generally regenerated through embryogenesis but can also be regenerated by shoot organogenesis.

Useful selectable marker genes are well known in the art and include, for example, herbicide and antibiotic resistance genes including but not limited to neomycin phosphotransferase II (conferring resistance to kanamycin, paramomycin and G418), hygromycin phosphotransferase (conferring resistance to hygromycin), 5-enolpyruvylshikimate-3-phosphate synthase (EPSPS, conferring resistance to glyphosate), phosphinothricin acetyltransferase (conferring resistance to phosphinothricin/bialophos), MerA (conferring resistance to mercuric ions). Selectable marker genes may be transformed using standard methods in the art.

The first step in the production of sugar cane plants containing novel genes involves delivery of DNA into a suitable plant tissue (described in the previous section) and selection of the tissue under conditions that allow preferential growth of any cells containing the novel genes. Selection is typically achieved with a selectable marker gene present in the delivered DNA, which may be a gene conferring resistance to an antibiotic, herbicide or other killing agent, or a gene allowing utilization of a carbon source not normally metabolized by plant cells. For selection to be effective, the plant cells or tissue need to be grown on selective medium containing the appropriate concentration of antibiotic or killing agent, and the cells need to be plated at a defined and constant density. The concentration of selective agent and cell density are generally chosen to cause complete growth inhibition of wild type plant tissue that does not express the selectable marker gene; but allowing cells containing the introduced DNA to grow and expand into adchromosomal clones. This critical concentration of selective agent typically is the lowest concentration at which there is complete growth inhibition of wild type cells, at the cell density used in the experiments. However, in some cases, sub-killing concentrations of the selective agent may be equally or more effective for the isolation of plant cells containing mini-chromosome DNA, especially in cases where the identification of such cells is assisted by a visible marker gene (e.g., fluorescent protein gene) present on the sugar cane mini-chromosome. Such sub-killing concentrations of the selective agent may be administered during part or all of the selection timing.

In some species (e.g., tobacco or tomato), a homogenous clone of modified cells can also arise spontaneously when bombarded cells are placed under the appropriate selection. An exemplary selective agent is the neomycin phosphotransferase II (nptII) marker gene, which is commonly used in plant biotechnology and confers resistance to the antibiotics kanamycin, G418 (geneticin) and paramomycin. In other species, or in certain plant tissues or when using particular selectable markers, homogeneous clones may not arise spontaneously under selection; in this case the clusters of modified cells can be manipulated to homogeneity using the visible marker genes present on the mini-chromosomes as an indication of which cells contain mini-chromosome DNA.

Regeneration of Modified Plants from Explants to Mature, Rooted Plants

For sugar cane, regeneration of a whole plant typically occurs via an embryogenic step that is not typically utilized for plant species where shoot organogenesis is more efficient. The explant tissue is cultured on an appropriate media for embryogenesis, and the embryo is cultured until shoots form. The regenerated shoots are cultured in a rooting medium to obtain intact whole plants with a fully developed root system. These plants are potted in soil and grown to maturity in a greenhouse.

Generally, regeneration and tissue culture of sugar cane plant parts and whole plants is challenging as sugar cane produces phenolic compounds while in culture. The present invention provides for methods of culturing sugar cane cells and tissues in media containing polyvinylpyrrolidone (PVP). The PVP acts as a sink for the phenolic compounds produced by sugar cane and enhances callus growth during selection as well as facilitating callus and plantlet regeneration. Furthermore, generation of sugar cane callus can be facilitated by delivering to the plant cells and/or tissues mini-chromosomes of the invention that contain auxin genes. The presence of the auxin genes will facilitate callus induction of the transformed tissue. The invention also provides for tissue culture methods which cycle between the liquid culture media and solid culture media in order to promote the frequency and the morphogenic competence of the regenerable sugar cane callus.

For plants that develop through shoot organogenesis, regeneration of a whole plant involves culturing of regenerable explant tissues taken from sterile organogenic callus tissue, seedlings or mature plants on a shoot regeneration medium for shoot organogenesis, and rooting of the regenerated shoots in a rooting medium to obtain intact whole plants with a fully developed root system. These plants are potted in soil and grown to maturity in a greenhouse.

Explants are obtained from any tissues of a plant suitable for regeneration. Exemplary tissues include hypocotyls, internodes, roots, cotyledons, petioles, cotyledonary petioles, leaves and peduncles, prepared from sterile seedlings or mature plants.

Explants are wounded (for example with a scalpel or razor blade) and cultured on a shoot regeneration medium (SRM) containing Murashige and Skoog (MS) medium as well as a cytokinin, e.g., 6-benzylaminopurine (BA), and an auxin, e.g., α-naphthaleneacetic acid (NAA), and an anti-ethylene agent, e.g., silver nitrate (AgNO₃). For example, 2 mg/L of BA, 0.05 mg/L of NAA, and 2 mg/L of AgNO₃ can be added to MS medium for shoot organogenesis. The most efficient shoot regeneration is obtained from longitudinal sections of internode explants.

Shoots regenerated via organogenesis are rooted in a MS medium. Plants are potted and grown in a greenhouse to sexual maturity for seed harvest.

In an exemplary method of regenerating a whole sugar cane plant with a sugar cane mini-chromosome, explants are pre-incubated for 1 to 7 days (or longer) on the shoot regeneration medium prior to bombardment with mini-chromosome (see below). Following bombardment, explants are incubated on the same shoot regeneration medium for a recovery period up to 7 days (or longer), followed by selection for transformed shoots or clusters on the same medium but with a selective agent appropriate for a particular selectable marker gene (described herein).

### Determination of Mini-Chromosome Structure and Autonomy in Sugar Cane Adchromosomal Plants and Tissues

The structure and autonomy of the sugar cane mini-chromosome in modified sugar cane plants and tissues can be determined by methods including but not limited to: conventional and pulsed-field Southern blot hybridization to genomic DNA from modified tissue subjected or not subjected to restriction endonuclease digestion, dot blot hybridization of genomic DNA from modified tissue hybridized with different mini-chromosome specific sequences, mini-chromosome rescue, exonuclease activity, PCR on DNA from modified tissues with probes specific to the mini-chromosome, or Fluorescence In Situ Hybridization to nuclei of modified cells. Table 2 below summarizes these methods.

TABLE 2
Examples of methods to determine mini-chromosome structure and autonomy

| Assay | Assay details | Potential outcome | Interpretation |
|---|---|---|---|
| Southern blot | Restriction digest of | Native sizes and patte [symbol] | Autonomous or |
| | genomic DNA* | of bands | integrated via CEN |
| | compared to purified | | fragment |
| | mini-C | Altered sizes or pattern | Integrated or rearrange [symbol] |
| | | of bands | |
| CHEF gel Southern | Restriction digest of | Native sizes and | Autonomous or |
| blot | genomic DNA | pattern of bands | integrated via CEN |
| | compared | | fragment |
| | to purified mini-C | Altered sizes or | Integrated or |
| | | pattern of bands | rearranged |
| | Native genomic DNA | Mini-C band | Autonomous circles |
| | (no digest) | migrating ahead of genon [symbol] | or linears present in |
| | | DNA | plant |
| | | Mini-C band | Integrated |
| | | co-migrating with | |
| | | genomic DNA | |
| | | >1 mini-C bands | Various possibilities |
| | | observed | |
| Exonuclease assay | Exonuclease digestion of | Signal strength close to | Autonomous circles |
| | genomic DNA followed | that w/o exonuclease | present |
| | by detection of circular | No signal or signal | Integrated |
| | mini-chromosome by | strength lower that w/o | |
| | PCR, dot blot, or | exonuclease | |
| | restriction digest | | |
| | optional), | | |
| | electrophoresis and | | |
| | southern blot (useful for | | |
| | circular mini- | | |
| | chromosomes) | | |
| Mini-chromosome | Transformation of plant | Colonies isolated only | Autonomous circles |
| rescue | genomic DNA into E. coli | from mini-C plants with | present, native mini-C |
| | followed by | mini-Cs, not from | structure |
| | selection for antibiotic | controls; mini-C | |
| | resistance genes on | structure matches that | |
| | mini-C | of the parental mini-C | |
| | | Colonies isolated only | Autonomous circles |
| | | from mini-C plants with | present, rearranged |
| | | mini-Cs, not from | mini-C structure OR |
| | | controls; mini-C | mini-Cs integrated via |
| | | structure different from | centromere fragment |
| | | parental mini-C | |
| | | Colonies observed both | Various possibilities |
| | | in mini-C-modified | |
| | | plants and in controls | |
| PCR | PCR amplification of | All mini-c parts detected | Complete mini-C |
| | various parts of the mini- | PCR | sequences present in |
| | chromosome | | plant |
| | | Subset of mini-c parts | Partial mini-C sequences |
| | | detected by PCR | present in plant |
| FISH | Detection of mini- | Mini-C sequences | autonomous |
| | chromosome sequences | detected, free of genome | |
| | in mitotic or meiotic | Mini-C sequences | integrated |
| | nuclei by fluorescence | detected, associated with | |
| | in situ hybridization | genome | |
| | | Mini-C sequences | Both autonomous and |
| | | detected, both free and | integrated mini-C |
| | | associated with genome | sequences present |
| | | No mini-C sequences | Mini-C DNA not visible |
| | | detected | by FISH |

*Genomic DNA refers to total DNA extracted from plants containing a mini-chromosome [symbol]
indicates data missing or illegible when filed

Furthermore, sugar cane mini-chromosome structure can be examined by characterizing mini-chromosomes ‘rescued’ from sugar cane adchromosomal cells. Circular sugar cane mini-chromosomes that contain bacterial sequences for their selection and propagation in bacteria can be rescued from a sugar cane adchromosomal plant or plant cell and re-introduced into bacteria. If no loss of these sequences has occurred during replication of the sugar cane mini-chromosome in plant cells, the mini-chromosome is able to replicate in bacteria and confer antibiotic resistance. Total genomic DNA is isolated from the sugar cane adchromosomal plant cells by any method for DNA isolation known to those skilled in the art, including but not limited to a standard cetyltrimethylammonium bromide (CTAB) based method (Current Protocols in Molecular Biology (1994) John Wiley & Sons, N.Y., 2.3). The purified genomic DNA is introduced into bacteria (e.g., E. coli) using methods familiar to one skilled in the art (for example heat shock or electroporation). The transformed bacteria are plated on solid medium containing antibiotics to select bacterial clones modified with sugar cane mini-chromosome DNA. Modified bacterial clones are grown up, the plasmid DNA purified (by alkaline lysis for example), and DNA analyzed by restriction enzyme digestion and gel electrophoresis or by sequencing. Because plant-methylated DNA containing methylcytosine residues will be degraded by wild-type strains of E. coli, bacterial strains (e.g. DH10B) deficient in the genes encoding methylation restriction nucleases (e.g. the mcr and mrr gene loci in E. coli) are suitable for this type of analysis. Sugar cane mini-chromosome rescue can be performed on any plant tissue or clone of plant cells comprising a mini-chromosome.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

### Example 1

### Methods of Detecting and Characterizing Mini-Chromosomes in Plant Cells or of Scoring Mini-Chromosome Performance in Plant Cells

### Identification of Candidate Centromere Fragments by Probing BAC Libraries

Sugar cane centromere clones can be identified from a large sugar cane genomic insert library such as a Bacterial Artificial Chromosome (BAC) library. Probes are labeled using nick-translation in the presence of radioactively labeled dCTP, dATP, dGTP or dTTP as in, for example, the commercially available Rediprime kit (Amersham) as per the manufacturer's instructions. Other labeling methods familiar to those skilled in the art could be substituted. The libraries are screened and deconvoluted. Sugar cane genomic clones can be screened by probing with small centromere-specific clones. Other embodiments of this procedure may involve hybridizing a library with other centromere sequences. Of the BAC clones identified using this procedure, a representative set can be identified as having high hybridization signals to some probes, and optionally low hybridization signals to other probes. These are selected, the bacterial clones grown up in cultures, and DNA prepared by methods familiar to those skilled in the art such as alkaline lysis. The DNA composition of purified clones can be surveyed using, for example, fingerprinting by digesting with restriction enzymes such as, but not limited to, HinfI or HindIII. In a representative embodiment the restriction enzyme cuts within the tandem centromere satellite repeat. A variety of clones showing different fingerprints can be selected for conversion into mini-chromosomes and inheritance testing. It can also be informative to use multiple restriction enzymes for fingerprinting or other enzymes which can cleave DNA.

### Fingerprinting Analysis of BACs and Mini-Chromosomes

Sugar cane centromere function may be associated with large tandem arrays of satellite repeats. To assess the composition and architecture of the centromere BACs, the candidate BACs can be digested with a restriction enzyme, such as HindIII, which cuts with known frequency within the consensus sequence of the unit repeat of the tandemly repeated centromere satellite. Digestion products can then be separated by agarose gel electrophoresis. Large insert clones containing a large array of tandem repeats will produce a strong band of the unit repeat size, as well as less intense bands at 2× and 3× the unit repeat size, and further multiples of the repeat size. These methods are well-known and there are many possible variations known to those skilled in the art.

### Determining Sequence Composition of Mini-Chromosomes by Shotgun Cloning/Sequencing, Sequence Analysis

To determine the sequence composition of the sugar cane mini-chromosome, the centromeric region of the sugar cane mini-chromosome can be sequenced. To generate DNA suitable for sequencing, sugar cane mini-chromosomes can be fragmented, for example by using a random shearing method (such as sonication, nebulization, etc). Other fragmentation techniques may also be used such as enzymatic digestion. These fragments can then be cloned into a vector (e.g., a plasmid) and sequenced. The resulting DNA sequence can be trimmed of poor-quality sequence and of sequence corresponding to the vector. The sequence can then be compared to known DNA sequences using an algorithm such as BLAST to search a sequence database such as GenBank.

To determine the consensus of the sugar cane satellite repeat in the sugar cane mini-chromosome, the sequences containing the satellite repeat can be aligned using a DNA sequence alignment program such as ContigExpress from Vector NTI. The sequences may also be aligned to previously determined repeats for that species. The sequences can be trimmed to unit repeat length using the consensus as a template. Sequences trimmed from the ends of the alignment can be realigned with the consensus and further trimmed until all sequences are at or below the consensus length. The sequences can then be aligned with each other. The consensus can be determined by the frequency of a specific nucleotide at each position; for example, if the most frequent base is three times more frequent than the next most frequent base, it can be considered the consensus.

Methods for determining consensus sequences are well known in the art, see, e.g., U.S. Pat. App. Pub. No. 20030124561; Hall & Preuss (2002). These methods, including DNA sequencing, assembly, and analysis, are well-known and there are many possible variations known to those skilled in the art. Other alignment parameters may also be useful such as using more or less stringent definitions of consensus. Methods of determining consensus sequences are also described in the working Examples herein.

### Non-Selective Mini-Chromosome Mitotic Inheritance Assays

The following list of assays and potential outcomes illustrates how various assays can be used to distinguish autonomous events from integrated events.

### Assay #1: Transient Assay

Sugar cane mini-chromosomes are tested for their ability to become established as chromosomes and their ability to be inherited in mitotic cell divisions. In this assay, sugar cane mini-chromosomes are delivered to sugar cane plant cells, for example suspension cells in liquid culture. The cells used can be at various stages of growth. Optionally, a population in which some cells are undergoing division can be used. The sugar cane mini-chromosome is then assessed over the course of several cell divisions, by tracking the presence of a screenable marker, e.g. a visible marker gene such as a fluorescent protein. sugar cane mini-chromosomes that are established and inherited well may show an initial delivery into many single cells; after several cell divisions, these single cells divide to form clusters of mini-chromosome-containing cells. Other exemplary embodiments of this method include delivering sugar cane mini-chromosomes to other mitotic cell types, including roots and shoot meristems.

### Assay #2: Non-Lineage Based Inheritance Assays on Modified Transformed Cells and Plants

Sugar cane mini-chromosome inheritance is assessed on modified cell lines and plants by following the presence of the mini-chromosome over the course of multiple cell divisions. An initial population of sugar cane mini-chromosome containing cells is assayed for the presence of the sugar cane mini-chromosome, by the presence of a marker gene, including but not limited to a fluorescent protein, a colored protein, a protein assayable by histochemical assay, and a gene affecting cell morphology. In the use of a DNA-specific dye, all nuclei are stained with a dye including but not limited to DAPI, Hoechst 33258, OliGreen, Giemsa YOYO, or TOTO, allowing a determination of the number of cells that do not contain the mini-chromosome. After the initial determination of the percent of cells carrying the sugar cane mini-chromosome, the remaining cells are allowed to divide over the course of several cell divisions. The number of cell divisions, n, is determined by a method including but not limited to monitoring the change in total weight of cells, and monitoring the change in volume of the cells or by directly counting cells in an aliquot of the culture. After a number of cell divisions, the population of cells is again assayed for the presence of the sugar cane mini-chromosome. The loss rate per generation is calculated by the equation:

Loss rate per generation=1−(F/I)^{1/n}

The population of sugar cane mini-chromosome-containing cells may include suspension cells, callus, roots, leaves, meristems, flowers, or any other tissue of modified plants, or any other cell type containing a mini-chromosome.

These methods are well-known and there are many possible variations known to those skilled in the art; they have been used before with human cells and yeast cells.

### Assay #3: Lineage Based Inheritance Assays on Modified Cells and Plants

Sugar cane mini-chromosome inheritance is assessed on cell lines and plants comprising sugar cane mini-chromosomes by following the presence of the sugar cane mini-chromosome over the course of multiple cell divisions. In cell types that allow for tracking of cell lineage, including but not limited to root or leaf cell files, trichomes, and leaf stomata guard cells, sugar cane mini-chromosome loss per generation does not need to be determined statistically over a population, it can be discerned directly through successive cell divisions.

In other manifestations of this method, cell lineage can be discerned from cell position, or methods including but not limited to the use of histological lineage tracing dyes, and the induction of genetic mosaics in dividing cells.

In one simple example, the two guard cells of the stomata are daughters of a single precursor cell. To assay sugar cane mini-chromosome inheritance in this cell type, the epidermis of the leaf of a sugar cane plant containing a sugar cane mini-chromosome is examined for the presence of the sugar cane mini-chromosome by the presence of a marker gene, including but not limited to a fluorescent protein, a colored protein, a protein assayable by histochemical assay, and a gene affecting cell morphology. The number of loss events in which one guard cell contains the sugar cane mini-chromosome (L) and the number of cell divisions in which both guard cells contain the sugar cane mini-chromosome (B) are counted. The loss rate per cell division is determined as L/(L+B). Other lineage-based cell types are assayed in similar fashion. These methods are well-known and there are many possible variations known to those skilled in the art; they have been used before with yeast cells (though, instead of observing the marker in stomates, a color marker was observed in yeast colonies).

Linear sugar cane mini-chromosome inheritance may also be assessed by examining leaf or root files or clustered cells in callus over time. Changes in the percent of cells carrying the sugar cane mini-chromosome will indicate the mitotic inheritance.

### Assay #4: Inheritance Assays on Modified Cells and Plants in the Presence of Chromosome Loss Agents

Any of the above three assays can be done in the presence of chromosome loss agents (including but not limited to colchicine, colcemid, caffeine, etopocide, nocodazole, oryzalin, trifluran). It is likely that an autonomous sugar cane mini-chromosome will prove more susceptible to loss induced by chromosome loss agents; therefore, autonomous mini-chromosomes should show a lower rate of inheritance in the presence of chromosome loss agents. These methods have been used to study chromosome loss in fruit flies and yeast; there are many possible variations known to those skilled in the art.

### Example 2

### Sugar Cane Centromere Discovery from Genomic DNA

### Identification of Sugar Cane Satellite Repeat Sequences

Centromere satellite repeats were amplified from sugar cane (Saccharum officinarum X Saccharum spontaneum or Saccharum officinarum) genomic DNA using standard PCR methods. Briefly, PCR reaction was carried under the following conditions: 1 cycle at 95° C. for 3 minutes, 10 cycles of 94° C. for 15 seconds, 55° C. for 15 seconds, and 72° C. for 30 seconds, and 25 cycles at 94° C. for 15 seconds, 52° C. for 15 seconds and 72° C. for 30 seconds, followed by 1 cycle at 72° C. for 5 minutes. The sequences of primers used for amplifying satellite repeats were: forward: 5′-gtcacccagcagttccatcgggtgc-3′ (SEQ ID NO:75 and reverse: 5′-actgctgggtgacgtggetcaagt-3′ (SEQ ID NO:76). After PCR, amplified satellite repeats were cloned into a standard cloning vector (pCR2; Invitrogen Corp.; Carlsbad, Calif.; USA). Colonies with insertions were cultured, DNA was extracted and sequenced. This PCR analysis identified 201 satellite sequences (see, SEQ ID NOS: 1-201 in international application PCT/US2010/043052).

To determine the consensus of the identified satellite repeat sequences, these sequences were aligned using a DNA sequence alignment program (CONTIGEXPRESS® from VECTOR NTI® (Invitrogen)). The sequences were trimmed to unit repeat length using the consensus as a template. Sequences trimmed from the ends of the alignment were realigned with the consensus and further trimmed until all sequences were at or below the consensus length. The consensus (SEQ ID NO:73) was determined by the frequency of a specific nucleotide at each position; if the most frequent base is three times more frequent than the next most frequent base, it was considered the consensus.

Consensus satellite repeat sequence:

(SEQ ID NO: 73)
ccyagsagtt ccatcgggtg cgtccaaaay gatttyygag
cctatggtac gttygrcgca aaccgtgcac ctatcttgca
tcaagabtag cactatctcc aaacggaccg aaacgagctt
ccacttgagc cacgtca

The sugar cane centromere specific retrotransposon sequence CRS (Centromere Retrotransposon in sugar cane—see Nagaki & Murata, Chromosome Research, 2005, 13:195-203) was PCR amplified and sequenced using primers located in different region of the CRS sequence. The PCR reaction was carried out as described above using the following primer sequences:

| (SEQ ID NO: 77) | |
|---|---|
| CRSF | 5′-gggaagtaca gggacgaaga gc-3′ |
| (SEQ ID NO: 78) | |
| CRSF1 | 5′-actaacaatg cacgggaagg-3′ |
| (SEQ ID NO: 79) | |
| CRSF2 | 5′-gtaggccatg gcagtttgat-3′ |
| (SEQ ID NO: 80) | |
| CRSF3 | 5′-aacacaccac ccaatccaat-3′ |
| (SEQ ID NO: 81) | |
| CRSF4 | 5′-ccaaacaagc gtgttatgat tgt-3′ |
| (SEQ ID NO: 82) | |
| CRSF5 | 5′-aggttatgtg cgtcagtctc ttag-3′ |
| (SEQ ID NO: 83) | |
| CRSF6 | 5′-ggcaaacctg ttgcatactt tag-3′ |
| (SEQ ID NO: 84) | |
| CRSF7 | 5′-accatgtcat aaaactgatg atg-3′ |
| (SEQ ID NO: 85) | |
| CRSR | 5′-tgcaaccaaa ccaaatcacc ag-3′ |
| (SEQ ID NO: 86) | |
| CRSR1 | 5′-caagcgaaca atctcacgaa-3′ |
| (SEQ ID NO: 87) | |
| CRSR2 | 5′-aaatcatcat cgtgcgcata-3′ |
| (SEQ ID NO: 88) | |
| CRSR3 | 5′-aacacaccac ccaatccaat-3′ |
| (SEQ ID NO: 89) | |
| CRSR4 | 5′-gaacgctcct tgatgacac-3′ |
| (SEQ ID NO: 90) | |
| CRSR5 | 5′-gtacccacta cgcaaatcaa cc-3′ |
| (SEQ ID NO: 91) | |
| CRSR6 | 5′-caacttcagt ttgaccatca gtt-3′ |

The sequence for CRS is set out as SEQ ID NO:74. The primer pairs CRSF 5′-gggaagtacagggacgaagagc-3′ (SEQ ID NO:77) and CRSR 5′-tgcaaccaaaccaaatcaccag-3′ (SEQ ID NO:85) can be used to amplify CRS from sugar cane genomic DNA.

### BAC Library Construction

A Bacterial Artificial Chromosome (BAC) library was constructed from sugar cane genomic DNA. The sugar cane genomic DNA was isolated from cultivar R570 (PI 504632), a hybrid between S. officinarum and S. spontaneum, and digested with the restriction enzymes Mbo I. These enzymes were chosen because they are methylation insensitive and therefore can be used to enrich BAC libraries for centromere DNA sequences.

### Probe Identification and Selection

Three groups of sugar cane repetitive genomic DNA, including specific centromere-localized sequences, were initially compiled as candidate probes for hybridization with the BAC libraries. Four probes were picked to interrogate the BAC libraries. These probes represent different groups of commonly found repetitive sequences in the sugar cane genome. The four probes were: SCEN, SCRM and High Methylation/Low Methylation (HiMe and LoMe). The SCEN and SCRM probes were each pooled PCR products. Probes were prepared and labeled with standard molecular methods. The HiMe and LoMe probes were pooled genomic DNA cut with a methylation-sensitive enzyme (BfuC1); large DNA fragments were isolated for the “HighMe” probe and small DNA fragments were isolated for the “LowMe” probe. Positives reported are for the HighMe probe.

### Library Interrogation and Data Analysis

The BAC clones from the libraries were spotted onto filters for further analysis. The filters were hybridized with each of the probes to identify specific BAC clones that contain DNA from the group of sequences represented by the probes. Hybridization conditions were: hybridization at 65° C. for 12-15 hours and washing three times for 15-90 minutes with 0.25×SSC, 0.1% SDS at 65° C. Other exemplary stringent hybridization conditions could be used, such as hybridization at 65° C. 0.5×SSC 0.25% SDS for 15 minutes, followed by a wash at 65° C. for a half hour.

A total of 18,453 BAC clones from the library was interrogated with each of the 4 probes (SCEN, SCRM, HiMe, and LoME), and the hybridization intensities of the BAC clones with each probe were examined to quantify hybridization intensity for each clone. Scores of 1 to 10 (based on the hybridization intensities, with 10 being the strongest hybridization) were assigned and entered into a spreadsheet for classification. The spreadsheet contained a total of 3 tables, 1 for each probe used in the interrogation (values from HiMe and LoMe) probes were entered in a single table for comparison. Each table contained the hybridization scores of each BAC clone from the Mbo I library, to one of the 4 probes. Data analysis found BACs that contained different groups of repetitive sequences.

### Classification and Selection of BAC Clones for Mini-Chromosome Construction

BAC clones containing centromeric/heterochromatic DNA were identified by their visual hybridization scores to different probes. The goal was to select BAC clones that contained a diverse set of various repetitive sequences. Seven classes of centromeric BAC clones were eventually chosen to cover the broadest possible range of centromeric/heterochromatic sequences for sugar cane mini-chromosome (MC) construction. 658 unique clones that hybridize with one or more of the probes were isolated from one filter, which comprised 18,432 clones. They fell into classes as set out in Table 3 below.

TABLE 3
Classification of Centromere containing BACs

Probe Hybridization Range

| Class | SCEN | SCRM | HiMe | # clones identified |
|---|---|---|---|---|
| I | + | | | 472 |
| II | | + | | 360 |
| III | | | + | 398 |
| IV | + | + | | 219 |
| V | + | | + | 343 |
| VI | | + | + | 166 |
| VII | + | + | + | 156 |

*Probes with significant hybridization signals as determined by visual scoring are indicated with a “+”

### Example 3

### Construction of Sugar Cane MCs Containing Genomic DNA

A subset of BAC clones identified in Example 2 were grown, and DNA was extracted for MC construction using a NUCLEOBOND® purification kit from Clontech Laboratories, Inc. (Mountain View, Calif.; USA). To determine the molecular weight of centromere fragments in the BAC libraries, a frozen sample of bacteria harboring a BAC clone was grown in selective liquid media, and the BAC DNA harvested using a standard alkaline lysis method. The recovered BAC DNA was restriction digested and resolved on an agarose gel. Centromere fragment size was determined by comparing to a molecular weight standard.

### Donor DNA Containing Gene Stacks for MC Construction

Several donor DNA plasmids containing gene stacks for testing of MCs in plant tissues were built, varying depending on the specific fluorescent protein marker used for detection of transgenic events. One set of MCs was built that contained the gene stack from donor plasmid CHROM5798, which genetic elements are set out in Table 4. Another set of MCs was built that contained the gene stack from donor plasmid CHROM5434, which genetic elements are the same as in donor plasmid CHROM5798 except that the nuclear localized GFP gene was replaced with an AmCyan (Clontech) fluorescent protein gene. Another set of MCs was built that contained the gene stack from donor plasmid CHROM5436, which genetic elements are the same as in donor plasmid CHROM5798, except that the nuclear localized GFP gene was replaced with a ZsGreen (Clontech) fluorescent protein gene.

TABLE 4
Donor Components of CHROM5798

| Genetic Element | Size (bp) | Location (bp) | Details |
|---|---|---|---|
| YAT1 yeast | 2000 | 6271-8270 | PCR amplified YAT1 promoter from chromosome I of |
| promoter | | | Saccharomyces cerevisiae for expression of NptII in sugar |
| | | | cane |
| Arabidopsis | 360 | 5898-6257 | PCR amplified Arabidopsis thaliana intron from UBQ10 gen [symbol] |
| UBQ10 Intron | | | (At4g05320) for stabilization of NptII gene transcript and |
| | | | increase protein expression level |
| NPTII | 795 | 5076-5870 | Neomycin phosphotransferase II plant selectable marker |
| Rps16A | 489 | 4524-5012 | Amplified from Arabidopsis thaliana 40S ribosomal protein |
| terminator | | | S16 (At2g09990) for termination of NptII gene |
| Bacterial | 817 | 3525-4341 | Bacterial kanamycin selectable marker |
| kanamycin | | | |
| Terminator 6 | 332 | 3049-3380 | Terminator 6 |
| AcGFP (nuc) | 831 | 2084-2914 | Nuclear localized green fluorescent protein. |
| UBQ10 Promoter | 2038 | 10-2047 | PCR amplified Arabidopsis thaliana promoter from UBQ10 |
| | | | gene (At4g05320) for stabilization of DsRedI gene transcript |
| | | | and increase protein expression level |
| LoxP | 34 | 8290-8319 and | Recombination site for Cre mediated recombination (Arenski |
| | | 10802-10831 | et al 1983, Abremski et al 1984) |

[symbol]

indicates data missing or illegible when filed

### Preparation of Donor DNA for Retrofitting

Cre recombinase-mediated exchange was used to construct sugar cane MCs by combining the sugar cane centromere fragments cloned in pBe1oBAC 11 with the donor plasmid CHROM5798 (Table 5). The recipient BAC vector carrying the sugar cane centromere fragment contained a loxP recombination site; the donor plasmid contained two such sites, flanking the sequences to be inserted into the recipient BAC.

Sugar cane MCs were constructed using a two-step method. First, the donor plasmid was linearized to allow free contact between the two loxP sites; in this step the backbone of the donor plasmid is eliminated. In the second step, the donor molecules were combined with sugar cane centromere BACs and were treated with Cre recombinase, generating circular sugar cane MCs with all the components of the donor and recipient DNA. Sugar cane MCs were delivered into E. coli and selected on medium containing kanamycin and chloramphenicol. Only vectors that successfully cre recombined and contained both selectable markers survived in the medium. To determine the molecular weight of the sugar cane centromere fragments in the sugar cane MCs, three bacterial colonies from each transformation event were independently grown in selective liquid media and the sugar cane MC DNA was harvested using a standard alkaline lysis method. The recovered sugar cane MC was restriction digested and resolved on an agarose gel. Sugar cane centromere fragment size was determined by comparison to molecular weight standards. When variation in sugar cane centromere size was noted, the sugar cane MC with the largest sugar cane centromere insert was used for further experimentation. All 84 MCs subjected to further testing had the features described in Table 5.

TABLE 5
MC constructs tested in sugar cane callus

| | | | | | Original | |
|---|---|---|---|---|---|---|
| | | Strength of | | | CEN | Donor |
| MC | | Satellite | Strength of | Strength of | fragment | plasmid |
| CHROM# | BAC name | signal | CRS signal | FISH signal | size (KB) | CHROM# |
| 5800 | 3G17 | L | L | n/a | 90 | 5798 |
| 5801 | 5B12 | H | N | good | 115 | 5798 |
| 5802 | 18E23 | H | M | good | 80 | 5798 |
| 5803 | 19H6 | M | H | good | 102 | 5798 |
| 5804 | 24L19 | L | M | good | 35 | 5798 |
| 5805 | 21L3 | M | M | good | 120 | 5798 |
| 5806 | 4H1 | H | N | good | 90 | 5798 |
| 5807 | 3O5 | M | H | good | 100 | 5798 |
| 5808 | 1K10 | L | H | good | 150 | 5798 |
| 5809 | 17H17 | H | H | good | 110 | 5798 |
| 5810 | 21A4 | H | H | good | 150 | 5798 |
| 5811 | 18F12 | H | H | good | 110 | 5798 |
| 5812 | 20H10 | H | H | n/a | 150 | 5798 |
| 5813 | 21B1 | H | H | good | 130 | 5798 |
| 5814 | 24J1 | H | H | good | 65 | 5798 |
| 5815 | 19J18 | H | H | good | 100 | 5798 |
| 5816 | 17B4 | H | L | good | 70 | 5798 |
| 5817 | 18P24 | H | L | good | 120 | 5798 |
| 5818 | 20A3 | H | L | n/a | 160 | 5798 |
| 5819 | 24F15 | H | L | n/a | 120 | 5798 |
| 5820 | 17C9 | H | M | good | 125 | 5798 |
| 5821 | 19J8 | H | N | n/a | 120 | 5798 |
| 5822 | 18C14 | H | N | good | 130 | 5798 |
| 5823 | 17P2 | H | N | good | 110 | 5798 |
| 5824 | 17M9 | H | N | good | 150 | 5798 |
| 5825 | 24J17 | H | N | n/a | 170 | 5798 |
| 5826 | 24C6 | H | N | good | 75 | 5798 |
| 5827 | 3I9 | L | H | good | 120 | 5798 |
| 5828 | 22H16 | L | H | n/a | 135 | 5798 |
| 5829 | 6C15 | L | H | good | 70 | 5798 |
| 5830 | 20C8 | L | L | good | 90 | 5798 |
| 5831 | 19K22 | L | M | n/a | 105 | 5798 |
| 5832 | 17E7 | L | M | good | 105 | 5798 |
| 5833 | 24M21 | L | M | n/a | 130 | 5798 |
| 5834 | 18J2 | M | H | good | 105 | 5798 |
| 5835 | 17N22 | M | H | n/a | 145 | 5798 |
| 5836 | 7E2 | M | H | good | 230 | 5798 |
| 5837 | 1L6 | M | H | n/a | 90 | 5798 |
| 5838 | 1P13 | M | H | good | 90 | 5798 |
| 5839 | 4H14 | M | L | good | 70 | 5798 |
| 5840 | 17E9 | M | L | n/a | 110 | 5798 |
| 5841 | 3P16 | M | L | good | 100 | 5798 |
| 5842 | 22I13 | M | L | n/a | 130 | 5798 |
| 5843 | 23F24 | M | L | n/a | 90 | 5798 |
| 5844 | 1K6 | M | L | good | 80 | 5798 |
| 5845 | 1P14 | M | L | good | 135 | 5798 |
| 5846 | 19H7 | M | M | n/a | 150 | 5798 |
| 5847 | 18F16 | M | M | n/a | 160 | 5798 |
| 5848 | 22D19 | M | M | n/a | 100 | 5798 |
| 5849 | 6C9 | M | M | n/a | 120 | 5798 |
| 5850 | 23I19 | M | N | good | 65 | 5798 |
| 5851 | 3F1 | M | N | n/a | 75 | 5798 |
| 5852 | 2A7 | M | N | n/a | 75 | 5798 |
| 5853 | 20A22 | N | N | none CEN | 100 | 5798 |
| 5854 | 19A22 | N | N | n/a | 115 | 5798 |
| 5855 | 17A22 | N | N | n/a | 120 | 5798 |
| 5856 | 21A22 | N | N | none CEN | 80 | 5798 |
| 5857 | 18A22 | N | N | n/a | 120 | 5798 |
| 5858 | 1A4 | L | H | n/a | 65 | 5798 |
| 5859 | 1M10 | N | N | n/a | 110 | 5798 |
| 5860 | 7D11 | M | M | n/a | 80 | 5798 |
| 5861 | 7J24 | M | L | n/a | 100 | 5798 |
| 5862 | 1P1 | H | L | n/a | 125 | 5798 |
| 5863 | 21B11 | H | N | good | 150 | 5798 |
| 5864 | 8O2 | M | L | good | 70 | 5798 |
| 5865 | 8I7 | M | H | good | 135 | 5798 |
| 5866 | 17H17 | H | H | good | 110 | 5434 |
| 5867 | 21A4 | H | H | good | 140 | 5434 |
| 5868 | 19J18 | H | H | good | 95 | 5434 |
| 5869 | 18P24 | H | L | good | 120 | 5434 |
| 5870 | 17C9 | H | M | good | 120 | 5434 |
| 5871 | 18E23 | H | M | good | 85 | 5434 |
| 5874 | 4H14 | M | L | good | 85 | 5434 |
| 5876 | 17P2 | H | N | good | 100 | 5434 |
| 5878 | 6C15 | L | H | good | 80 | 5434 |
| 5881 | 17C9 | H | M | good | 120 | 5436 |
| 5882 | 17P2 | H | N | good | 100 | 5436 |
| 5883 | 4H14 | M | L | good | 85 | 5436 |
| 5884 | 18E23 | H | M | good | 85 | 5436 |
| 5885 | 6C15 | L | H | good | 80 | 5436 |
| 5886 | 18P24 | H | L | good | 120 | 5436 |
| 5887 | 19J18 | H | H | good | 95 | 5436 |
| 5888 | 21A4 | H | H | good | 140 | 5436 |
| 5889 | 17H17 | H | H | good | 110 | 5436 |

H indicates high hybridization signal in the original filter hybridization, M indicates medium signal, and L indicates low signal. N indicates no signal was observed. In column labeled “Strength of FISH signal,” “good” indicates strong hybridization to centromeres observed in root tip spread, n/a indicates “not determined,” and “none CEN” means no hybridization was observed to the centromeric region of any chromosomes.

### Example 4

### MC Delivery into Sugar Cane Cells and Regeneration

The sugar cane MCs from Example 3 were tested in several sugar cane cells, including Saccharum officinarum and a hybrid between S. officinarum and S. spontaneum, and the procedure was optimized for antibiotic selection, cell pre-treatments, and bombardment conditions. MCs were tested both in leaf-roll tissue directly, or callus tissue that was initiated from leaf-rolls. The presence of MCs was determined both by direct molecular assays or indirect measurement of fluorescent cells. Preliminary results identified several MCs that successfully generated fluorescent cell clusters in Saccharum cells.

### Sugar Cane Transformation, Selection and Regeneration.

Prior to delivery of the two-gene stack containing MCs from Example 3, sugar cane callus was initiated from leaf roll tissue. Sugar cane tops were collected from greenhouse-grown plants for preparing explants. The sugar cane tops (minimally 3-6 months old) were cut below the highest visible node. The older leaves (approx 3-4) were removed until the internode was visible and cut about 2″ below this internode, and disinfected by submerging in 20% bleach for 20 min (5-10 tops in 3 L bleach solution). Subsequently, the cane tops were rinsed with sterile distilled water 3-4 times to remove excess bleach. In a tissue culture hood, more external leaves were removed from the cane tops, and the top portion was cut off leaving about 10 to 12 cm above the internode and 1 cm below the internode. Thin stem sections (approx 1 to 1.5 mm thickness) were sliced from the lower edge of the internodes. During this process, tools were frequently dipped in antioxidant mixture (PhytoTechnology Laboratories; Shawnee Mission, Kans.; USA) to avoid browning at cut sites. Those sections with orange centers were avoided, and only those sections with green centers were used for callus induction. Approximately 9 pieces of thin stem sections were placed per plate on MS3 Medium (Murashige and Skoog, 1962. Physiologia Plantarum, 15:472-497), supplemented with 500 mg/L casein hydrolysate, 20 g/L sucrose and 3 mg/L 2,4-D, pH to 5.8 and solidified with 2.5 g/L GELRITE® (Sigma-Aldrich; Saint Louis, Mo.; USA) or 6 g/L Phytoblend (Caisson Laboratories; North Logan, Utah; USA). The callus was sub-cultured once after a 15-day interval onto the same medium or MS1 (MS 1; 4.3 g/l MS salts and vitamins supplemented with 20 g/l sucrose, 0.5 g/l casein, 1 mg/l 2,4-D, pH to 5.8) medium. Callus was generally sufficiently established for bombardment after 2-3 months. Prior to bombardment with MCs, the white, nodular and embryogenic calli were subcultured for 3-4 days on MS3 Medium, and then transferred onto sugar cane Osmotic Medium (SCOM) prior to bombardment (4-5 hours at 28° C.). Sugar Cane Osmotic Medium consists of MS3 medium supplemented with 500 mg/L casein hydrolysate, 20 g/L sucrose and 3 mg/L 2,4-D. pH to 5.8 and solidify with 2.5 g/L GELRITE® or 6 g/L Phytoblend with the addition of 36.4 g/L sorbitol, and 36.4 g/L mannitol.

Precipitation of MC DNA onto gold particles for the purpose of delivery using the biolistic method was performed as follows: 1.8 mg of sterile, washed gold (0.6 μM diameter was preferred) was combined with desired amount of MC DNA (in 1×TE). Careful handling of DNA was critical; wide bore tips were used for all pipetting, and solutions were preferentially dispensed into the bottom of the tube to assist with the gentle mixing process. The volume was brought to 250 μA with cold (4° C.) sterile water and 250 μl of cold (4° C.) 2.5M CaCl₂ was added immediately, followed by addition of 50 μl of filter sterilized 0.1M Spermidine (free base, filter sterilized). The mixture was gently finger vortexed 1-2× to ensure even mixing of all solutions, and DNA was allowed to precipitate onto the gold particles on ice for 1.5 hours; with finger vortexing 1-2× after 45 min. The gold/DNA mixture was pelleted (5 min, 800 rpm, RT) and washed once with 100% ethanol, and 36 μl 100% ethanol was added to the gold/DNA pellet, and mixed gently. Typically 6 μl of gold/DNA/ethanol was used per macrocarrier (i.e., one bombardment shot). The absolute number of molecules delivered per shot was varied by precipitating a varying amount of DNA onto the gold particles.

Bombardment conditions using the BioRad PDS-1000/He biolistic transformation system Bio-Rad Laboratories; Hercules, Calif.) were as follows. A rupture disk rating of 900-1800 psi; 1100 or 1300 psi was preferred, with one shot per plate. The preferred gap distance (distance from rupture disk to macrocarrier) was 6 mm. The target shelf for tissue was L2-L4; L2 (3rd shelf from the bottom) was preferred. Vacuum pressure of 27.5-28 in Hg; 27.5 in Hg was preferred. These bombardment conditions were tested with R570 callus, other conditions (pressure, rupture disk rating, gap distance, target shelf, duration of osmotic and rest treatments, etc.) can of course be modified for other genotypes and/or tissues. Following bombardment, callus was allowed to recover at 28° C. in the dark for an additional 12-18 hours on SCOM. Tissues were transferred onto MS3 medium for recovery for 3-4 days, and the number of calli present on each plate used for bombardment was counted. At the end of the recovery period, calli were checked for transient expression of fluorescent marker genes under the microscope, and the transient transformation efficiency was calculated.

Transient expression of MC encoded green fluorescent protein (GFP) gene AcGFPnuc was demonstrated in sugar cane (cv. L97-128) callus induced from immature leaf tissue. Approximately 2.5×10⁹ DNA molecules for 9 different MC DNAs were delivered into the callus tissues 4 hours after osmotic treatment per plate, and the bombarded tissues were examined for GPF expression 4 days later. The results are summarized in Table 6. Calli expressing GFP were observed in 6 out of the 9 MCs delivered in this experiment, with a frequency ranging from 1.7% for MC CHROM5889 to 20% for MC CHROM5886. However, no GFP expressing calli were observed for 3 MC constructs. These results demonstrated that MC DNA had been successfully delivered into sugar cane callus cells and the MC encoded fluorescent protein gene AcGFPnuc was expressed and biologically functional.

TABLE 6
Transient expression of AcGFPbuc in sugar cane MC
containing transgenic callus

| | | | | # of calli | % GFP |
|---|---|---|---|---|---|
| | BAC | # of plates | # of calli | expressing | expressing |
| MC # | Cen | observed | observed | GFP | callus |
| 5881 | 17C9 | 7 | 140 | 5 | 3.6 |
| 5882 | 17P2 | 9 | 92 | 4 | 4.3 |
| 5883 | 4H14 | 12 | 136 | 0 | 0.0 |
| 5884 | 18E24 | 12 | 140 | 6 | 4.3 |
| 5885 | 6C15 | 10 | 120 | 3 | 2.5 |
| 5886 | 18P24 | 4 | 60 | 12 | 20.0 |
| 5887 | 19J18 | 5 | 82 | 0 | 0.0 |
| 5998 | 21A4 | 5 | 94 | 0 | 0.0 |
| 5889 | 17H17 | 6 | 120 | 2 | 1.7 |

For selection of transgenic events, bombarded calli were transferred onto sub-lethal selection medium (ChromMS3G30), and culture at 28° C., in the dark, for 2 weeks. ChromMS3G30 medium consists of MS3 medium supplemented with 30 mg/l G418 sulfate (Geneticin (Sigma)) after autoclaving. The calli were broken up into small pieces and transferred onto lethal selection medium (second round of selection) MS3G50, and culture at 28° C., in the dark, for 4 weeks. MS3G50 Medium consists of MS3 medium supplemented with 50 mg/l G418 sulfate after autoclaving. Tissue growth was visually assessed to identify resistant callus. Resistant calli were subcultured for another round of selection on ChromMS3G50 for an additional 4 weeks.

For plant regeneration, surviving calli (putative resistant calli) were transferred onto RSCG25 medium to initiate regeneration and were cultured at 26° C., low light (16 hour day length, 26° C.) for 3-4 weeks. RSCG25 Medium consists of MS3 medium supplemented with 500 mg/L casein hydrolysate, 20 g/L sucrose and 0.5 mg/L kinetin, pH to 5.8 and solidified with 2.5 g/L GELRITE®, and further supplemented with 25 mg/L G418 sulfate after autoclaving. Developing plantlets were transferred to RtSC medium in sundae cups (Solo Cup Company; Lake Forest, Ill.; USA) for plantlet growth and root development and cultured at 26° C., 16 hour day length. RtSC medium consists of MS medium supplemented with 25 g/L sucrose, pH to 5.8 and solidify with 2.5 g/L GELRITE®, further supplemented with 20 mg/L G418 sulfate after autoclaving. Finally, plantlets were transferred into pre-moistened soil-less mix (LC1, BFG Supply Company; Joliet, Ill.; USA) under a humidome in an 18-well flat in a growth chamber (28° C., 16 hour day length). The dome was cracked open slightly to slowly reduce humidity 3-4 days after transplanting. The dome was removed completely 2 days later and plantlets were transferred to a greenhouse (28° C., 16 hour day length). Plants were watered from trays beneath the pots when the soil began to dry.

### Identification of MC Containing Transgenic Events

More than 1200 putative transgenic sugar cane events were generated from mini-chromosome transformation. A total of 920 (˜76%) of putative transgenic calli events were analyzed using diagnostic PCR for the several DNA fragments carried on the gene stack. The presence or absence of the nptII, AcGFPnuc or ZsGreen (depending on the mini-chromosome used in transformation), and the ubiquitin (UBQ10) promoter was determined and compared to amplification of the endogenous genomic internal control ADH. The events thus screened cover a collection of 51 MCs with an average size ranging between 65 and 187 kb.

Transgenic events were derived from the bombardment of six different sugar cane genotypes (R570, L97-128, Q117, NCo310, Pindar and Q63). Of the 920 events, 33% were derived from the callus bombarded with the DNA concentration of 1×10⁹ molecules (90-200 ng of DNA) per shot, 8.7% from 2.5×10⁹ molecules (200-500 ng of DNA), 37.6% from 5×10⁹ molecules (450 ng-1 μg of DNA) and 6.2% from 1×10¹⁰ molecules (800 ng-1.2 μg of DNA) per shot. The putative transgenic events were obtained after selecting the bombarded calli on different levels of G418 concentration ranging from 22.5 mg to 50 mg/l (100% potency) for a period of 4-5 months with a minimum of 4 rounds of selection. For PCR screening, total genomic DNA was isolated from approximately 40-60 mg of callus tissue by the DNA preparation method of Krysan et al. (Krysan P H, Young J C, Tax F, Sussman M R (1996) Identification of transferred DNA insertions within Arabidopsis genes involved in signal transduction and ion transport. Proc Natl Acad Sci USA 93: 8145-8150). The concentration of DNA was measured, normalized uniformly and used for either single-step or multiplex PCR analysis. The optimized PCR conditions, primers and reagents that were used for detecting the 4 PCR fragments in all the calli events generated is as follows.

Single-step PCR conditions used an initial denaturation at 95° C. for 2 minutes, followed by 35 cycles each consisting of 0.3 minutes denaturation at 94° C., 0.3 minutes annealing at 52° C., and 1.2 minutes extension at 72° C., followed by a final extension for 4 minutes at 72° C., after which the samples were kept at 4° C. indefinitely. The PCR reaction was performed in a total volume of 25 μl, consisting of 19.2 μl water, 2.5 μl 10X NEB Buffer, 0.4 μl dNTP mix (40 Mm), 0.2 μl F-Primer (20 μM), 0.2 μl R-primer (20 μM), 0.50.2 μl NEB Taq Polymerase and 2 μl DNA (100-200 ng). Multiplex PCR conditions used an initial denaturation at 95° C. for 1 minutes, followed by 40 cycles each consisting of 0.1 minutes denaturation at 95° C., 0.1 minutes annealing at 55° C. (and increasing 0.1° C. with each subsequent cycle), and 1.5 minutes extension at 72° C., followed by a final extension for 5 minutes at 72° C., after which the samples were kept at 4° C. indefinitely. The PCR reaction was performed in a total volume of 25 μl, consisting of 15 μl water, 5 μl 5× Multiplex Master Mix (New England Biolabs; Ipswich, Mass.; USA), 2 μl F-Primer (20 μM), 2 μl R-primer (20 μM), and 1 μl DNA (100-200 ng). The expected PCR product sizes were 470 bp (ADH), 662 bp (AcGFPnuc), 886 bp (UBQ10), 1000 bp (nptII), and 924 bp (ZsGreen). The PCR reaction was carried out as described above using the following primer sequences:

(SEQ ID NO: 92)

| | | |
|---|---|---|
| ADH1 | CHSL-285 | 5′-aagtcggcag agagcaacat-3′ |

(SEQ ID NO: 93)

| | | |
|---|---|---|
| ADH1 | CHSL-286 | 5′-cagatgcaaa cccaacacac-3′ |

(SEQ ID NO: 95)

| | | |
|---|---|---|
| AcGFPnuc | CHSL-132 | 5′-cgattttctg ggtttgatcgtt |
| | | ag-3′ |

(SEQ ID NO: 96)

| | | |
|---|---|---|
| AcGFPnuc | CHSL-199 | 5′-cattgtgggc gttgtagttg-3′ |

(SEQ ID NO: 97)

| | | |
|---|---|---|
| UBQ10 | CHSL-468 | 5′-gttgtggttg gtgctttcct-3′ |

(SEQ ID NO: 98)

| | | |
|---|---|---|
| UBQ10 | CHSL-469 | 5′-ccactttgac gccgtttatt-3′ |

(SEQ ID NO: 98)

| | | |
|---|---|---|
| NPTII | CHSL-132 | 5′-cgattttctg ggtttgatcgtt |
| | | ag-3′ |

(SEQ ID NO: 99)

| | | |
|---|---|---|
| NPTII | CHND7 | 5′-gaactcgtca agaaggcgata-3′ |

(SEQ ID NO: 100)

| | | |
|---|---|---|
| ZsGreen | CHSL-132 | 5′-cgattttctg ggtttgatcgtt |
| | | ag-3′ |

(SEQ ID NO: 101)

| | | |
|---|---|---|
| ZsGreen | CHSL-201 | 5′-tcagggcaat gcagatcc-3′ |

Among the 920 events analyzed, 358 events (38.9%) showed the presence of all three PCR amplicons (nptII, AcGFPnuc or ZsGreen, and UBQ10) (summarized in Table 7). Based on this analysis the escape frequency is around 61%. The observed escape rate appears the highest in genotype R570, in which most of the transformations were performed.

TABLE 7
Sugar cane MC containing transgenic callus

| | | Positive events |
|---|---|---|
| | | for all |
| MC # | Events analyzed | amplicons |
| 5800 | 24 | 12 |
| 5801 | 9 | 0 |
| 5802 | 42 | 18 |
| 5803 | 5 | 1 |
| 5804 | 10 | 2 |
| 5805 | 3 | 1 |
| 5806 | 4 | 1 |
| 5809 | 11 | 3 |
| 5810 | 7 | 5 |
| 5812 | 4 | 0 |
| 5814 | 88 | 62 |
| 5816 | 29 | 14 |
| 5817 | 42 | 12 |
| 5819 | 75 | 43 |
| 5820 | 53 | 42 |
| 5821 | 4 | 1 |
| 5822 | 18 | 1 |
| 5823 | 4 | 0 |
| 5824 | 12 | 0 |
| 5825 | 7 | 0 |
| 5827 | 6 | 0 |
| 5830 | 5 | 0 |
| 5834 | 20 | 3 |
| 5835 | 4 | 1 |
| 5837 | 12 | 1 |
| 5839 | 7 | 0 |
| 5840 | 3 | 0 |
| 5842 | 2 | 0 |
| 5844 | 7 | 0 |
| 5846 | 14 | 0 |
| 5850 | 14 | 11 |
| 5851 | 14 | 0 |
| 5852 | 5 | 0 |
| 5854 | 26 | 3 |
| 5856 | 11 | 3 |
| 5857 | 6 | 1 |
| 5858 | 25 | 1 |
| 5859 | 14 | 0 |
| 5860 | 26 | 4 |
| 5862 | 11 | 0 |
| 5863 | 11 | 0 |
| 5864 | 50 | 8 |
| 5873 | 2 | 2 |
| 5874 | 14 | 8 |
| 5881 | 47 | 44 |
| 5882 | 19 | 13 |
| 5883 | 12 | 4 |
| 5884 | 35 | 16 |
| 5885 | 23 | 8 |
| 5888 | 2 | 2 |
| 5889 | 22 | 7 |
| Total | 920 | 358 |

MC transgenic events were obtained in all genotypes tested, and significant genotype dependence was noted for both transformation efficiency and escape rate for G418 selection. Table 8 summarizes transformation results for several genotypes.

TABLE 8
Sugar cane MC transgenic events in multiple genotypes

| | No. of putative | No. of PCR | Escape frequency |
|---|---|---|---|
| Genotype | events produced | positive events | (%) |
| R570 | 554 | 94 | 83 |
| Q117 | 151 | 95 | 37 |
| L97-128 | 182 | 142 | 22 |
| NCo310 | 6 | 6 | 0 |
| Pindar | 23 | 18 | 21.7 |
| Q63 | 4 | 3 | 25 |
| Total | 920 | 358 | |

### Evaluation of Autonomous MCs

To evaluate whether the candidate sugar cane MCs were maintained autonomously, fluorescence in situ hybridization (FISH) was performed on mitotic metaphase chromosome spreads from callus tissue. FISH was performed essentially as described in Kato et al. Proc. Natl. Acad. Sci. U.S.A. 101: 13554-13559, 2004, using probes labeled with ALEXA FLUOR® 488 (“Alexa488”) and ALEXA FLUOR®568 (“Alexa568;” Invitrogen). Alexa488 labeled CHROM5798 DNA was used as a MC-specific probe: Alexa568 labeled pBeloBAC11 DNA was used as a second MC-specific probe. Alexa568 labeled PCR amplified centromere sequences from BAC 18E23 were used as a centromere-specific probe. The latter probe was also expected to stain centromere regions on the endogenous chromosomes.

For FISH evaluation, freshly growing callus tissue was collected following a recent transfer to fresh media. Depending on genotype, different morphologies were apparent. Generally, tissue was nodular and firm, and met forceps with resistance. Using forceps or scalpel, a very small cluster of nodules was excised and transferred to a 1.7 mL microfuge tube. A few microliters of dH₂O were added to the tube to keep the tissue moist. The tube was covered with cap containing a small puncture to allow exposure to nitrous oxide in next step. Callus tissue was placed in the pressure chamber under 160 psi for 4.5 hours. Tissue was fixed in 90% acetic acid, and spread onto poly-lysine coated glass slides by squashing thin cross sections. Following hybridization, slides were counter-stained with DAPI (0.04 mg/ml) and ≧15 metaphase cells were evaluated per callus using a Zeiss Axio-Imager (Carl Zeiss Microlmaging, Inc.; Thornwood, N.Y.; USA) equipped with rhodamine, FITC, and DAPI filter sets (excitation BP 550/24, emission BP 605/70; excitation BP 470/40, emission: BP525/50; and excitation G 365, emission BP 445/50, respectively). Gray-scale images were captured in each panel, merged and adjusted with pseudo-color using Zeiss AxioVision (Version 4.5; Carl Zeiss Microlmaging, Inc.) software; fluorescent signals from doubly-labeled MCs were detected in both the red and green channels.

Extra-chromosomal signals were considered to indicate autonomous sugar cane MCs if the images showed co-localization of the Alexa488 (green) and Alexa568 (red) signals within 1 nuclear diameter of the endogenous metaphase sugar cane chromosomes, and the signals were clearly distinct from the DAPI-stained host chromosomes. Typical Autonomous MC signals in FISH hybridization show overlapping distinct Alexa488 (green) and Alexa568 (red) signals that in computer-generated merged images, the overlap shows a yellow signal. Integrated constructs result in two distinct FISH signals, each on a replicated metaphase chromatid, and usually these FISH signals do not overlap with the centromere region. Autonomous MCs were found to co-exist in the presence of integrated constructs, indicating the ability of a specific MC to produce a purely autonomous event in transgenic lines obtained in parallel with the event characterized here, or obtained from future transformation experiments under different transformation conditions. Table 9 summarizes the preliminary FISH evaluation of selected transgenic lines that were confirmed to contain MCs identified by PCR, and demonstrated that both autonomous only (category A) and autonomous and integrated events (category A+I) were obtained for a significant number of sugar cane MCs.

TABLE 9
Preliminary FISH evaluation of sugar cane MC transgenic events

| | | | Integrated | |
|---|---|---|---|---|
| MC# | Event ID# | Autonomous copy | copy | Category |
| 5802 | RC5802-85-12-5 | + | − | A |
| 5802 | RC5802-85-14-2 | + | − | A |
| 5802 | RC5802-85-14-2 | + | − | A |
| 5802 | RC5802-85-16-1 | + | − | A |
| 5814 | BC5814-109-6-1 | + | − | A |
| 5814 | BC5814-109-9-1 | + | − | A |
| 5819 | AC5819-109-29-1 | + | − | A |
| 5819 | AC5819-109-30-2 | + | − | A |
| 5820 | BC5820-105-13-1 | + | − | A |
| 5820 | BC5820-105-13-1 | + | − | A |
| 5820 | RC5820-74-6-3 | + | − | A |
| 5824 | RC5824-77-29-1 R | + | − | A |
| 5837 | RC5837-88-13-1 | + | − | A |
| 5840 | RC5840-99-10-1 | + | − | A |
| 5802 | RC5802-85-14-5 | + | + | A + I |
| 5809 | RC5809-79-4-1 | + | + | A + I |
| 5810 | BC5810-112-23-1 | + | + | A + I |
| 5810 | BC5810-112-23-2 | + | + | A + I |
| 5814 | AC5814-109-12-2 | + | + | A + I |
| 5814 | AC5814-109-14-1 | + | + | A + I |
| 5814 | AC5814-109-14-8 | + | + | A + I |
| 5814 | AC5814-109-15-10 | + | + | A + I |
| 5814 | AC5814-109-15-5 | + | + | A + I |
| 5814 | RC5814-101-16-1 | + | + | A + I |
| 5816 | BC5816-109-21-5 | + | + | A + I |
| 5816 | BC5816-109-21-6 | + | + | A + I |
| 5817 | PC5817-104-1-1 | + | + | A + I |
| 5817 | PC5817-104-2-1 | + | + | A + I |
| 5817 | PC5817-104-5-1 | + | + | A + I |
| 5819 | AC5819-109-26-1 | + | + | A + I |
| 5819 | AC5819-109-27-1 | + | + | A + I |
| 5819 | AC5819-109-29-1 | + | + | A + I |
| 5819 | AC5819-109-29-4 | + | + | A + I |
| 5819 | BC5819-105-3-023 | + | + | A + I |
| 5820 | BC5820-105-11-1 | + | + | A + I |
| 5820 | BC5820-105-15-1 | + | + | A + I |
| 5820 | BC5820-105-15-1 | + | + | A + I |
| 5820 | BC5820-105-15-1 | + | + | A + I |
| 5834 | RC5834-80-2-1 | + | + | A + I |
| 5850 | RC5850-74-16-2 | + | + | A + I |
| 5850 | RC5850-74-16-2 | + | + | A + I |
| 5850 | RC585074-16-2 | + | + | A + I |
| 5850 | RC5850-74-18-8 | + | + | A + I |

Events with high quality FISH signals as determined by visual scoring are indicated with a “+”, a clear absence of signal is indicated by “−”.

### Example 5

### Sequencing and Bioinformatic Analysis of Sugar Cane Mini-Chromosome BACs

Eleven of the mini-chromosome BAC clones from Table 5 were grown under standard conditions and BAC DNA was isolated from the E. coli host cells using the alkaline lysis protocol and purified by cesium chloride centrifugation. Thirty micrograms of BAC DNA was used to produce whole BAC shotgun sequencing libraries by Amplicon Express (Pullman, Wash.). GS-FLX Titanium libraries were made using MIDs (multiplex identifiers) according to the Roche GS-FLX Titanium Rapid Preparation Kit (version October 2009). The constructed libraries were pooled and subjected to a GS-FLX Titanium sequencing run.

After the run was complete, the data were sorted into separate libraries for each BAC clone based on the MID tags. Before assembly, all E. coli reads were removed. BAC sequences were de novo assembled using “Newbler” (454 runAssembly software, Software

Release: 2.3). The minimum identity was set to both 90% and 98%. The National Center for Biotechnology Information (NCBI) UniVec database was used to screen all generated contigs for traces of contaminating vector sequences. The vector sequences were replaced by “x” in the “screen files”. This step also makes it possible to determine the ends of the sequenced BACs.

The contigs assembled with the Newbler software were screened to eliminate vector sequences from the donor plasmid and the pBe1oBAC11 vector (see Example 3). The screening was performed using the program cross_match version 0.990329 (phrap/cross_match/swat package from University of Washington). Thereafter, the assembled contigs were formatted separately using the utility program formatdb (NCBI, download from ftp://ftp.ncbi.nih.gov/blast/) to form unique databases from each of the 11 assemblies as required for use of the BLAST software package.

The sequences for the sugar cane CEN satellite consensus monomer (SEQ ID NO:73) and maize CRM1 (NCBI Accession Number AC116034.3) and maize CRM2 (NCBI Accession Number AY129008.1) centromeric retro elements were used as “query sequences” to identify similar elements in the assemblies from the 11 sugar cane MCs by executing the blastn algorithm from the BLAST software (NCBI, download from ftp://ftp.ncbi.nih.gov/blast/). The 137 bp sugar canecane satellite repeat consensus sequence (SEQ ID NO:73) was generated as described in Example 2 bp comparing 201 PCR amplified, cloned and sequenced sugar cane centromere satellite repeat monomers.

The criteria used to filter were different for the satellite sequences (at least 70% or greater in homology and at least 100 nucleotides in the alignment length) and the CRM-like sequences (at least 70% or greater in homology and at least 900 nucleotides in the alignment length). The alignment sequences, hit coordinates and homology percentages were parsed and loaded into a MySQL database. SQL queries were run to extract the monomer fragments as well as the CRM-like sequences from the assembled contigs. Multiple consensus sequences for each element from each of the MCs were identified by first binning the sequences into groups based on percent homology, and then generating consensus sequences for each group.

The assemblies from the 11 sugar cane MCs were also screened to identify additional repeats in the sugar cane mini-chromosome BACs using the online Plant Repeat Databases maintained by Michigan State University at plantrepeats.plantbiology.msu.edu. Since there is no repeat database specifically designed for sugar cane, the repeat sequence associated with sorghum was used to search for any possible repeats in the assemblies. The identified sequences from the assemblies were then used to query the NCBI EST database to generate compositional information for each of the assemblies. A summary of this information as well as statistical analysis related to each MC is shown below in Table 10.

TABLE 10

| Mini-chromosome | | | | | | | | | | | | |
|---|---|---|---|---|---|---|---|---|---|---|---|---|
| CHROM# | 5802 | 5809 | 5810 | 5814 | 5817 | 5819 | 5820 | 5823 | 5829 | 5834 | 5839 | 5824 |
| 1 Sequence and assembly | | | | | | | | | | | | |
| quality statistics | | | | | | | | | | | | |
| i. Total assembled | 85,188 | 96,128 | 131,539 | 43,800 | 103,112 | 136,868 | 82,991 | 20,822 | 83,336 | 108,219 | 69,043 | 39,057 |
| sequence length without | | | | | | | | | | | | |
| vector sequence | | | | | | | | | | | | |
| 2 Centromere specific | | | | | | | | | | | | |
| repeat content-Satellite, | | | | | | | | | | | | |
| CRM1 and CRM2 | | | | | | | | | | | | |
| a. % of BAC insert | 77.18% | 33.93% | 64.68% | 31.75% | 45.98% | 53.23% | 55.58% | 27.37% | 44.03% | 58.89% | 80.38% | 33.65% |
| composed of SCEN | | | | | | | | | | | | |
| repeats (Satellite) | | | | | | | | | | | | |
| b. Total assembled size | 65,748 | 32,616 | 85,079 | 13,907 | 47,411 | 72,855 | 46,126 | 5,699 | 36,693 | 63,730 | 55,497 | 13,144 |
| of SCEN repeats (2aX1i) | | | | | | | | | | | | |
| c. % of BAC insert | 5.07% | 5.77% | 12.31% | 16.50% | 0.00% | 0.66% | 6.51% | 0.00% | 9.72% | 3.73% | 1.15% | 0.00% |
| composed of CRS | | | | | | | | | | | | |
| retroelement (CRM1% + | | | | | | | | | | | | |
| CRM2%) | | | | | | | | | | | | |
| d. Total assembled size | 4,319 | 5,547 | 16,192 | 7,227 | 0 | 903 | 5,403 | 0 | 8,100 | 4,037 | 794 | 0 |
| of CRS elements | | | | | | | | | | | | |
| (2b + CRM1 + CRM2) | | | | | | | | | | | | |
| e. Total centromere | 70,067 | 38,163 | 101,272 | 21,134 | 47,411 | 73,758 | 51,529 | 5,699 | 44,793 | 67,767 | 56,291 | 13,144 |
| specific repeat content | | | | | | | | | | | | |
| (2b + 2d) | | | | | | | | | | | | |
| 3 Other repeats | | | | | | | | | | | | |
| a. % of other repeats | 0.96% | 16.29% | 2.32% | 12.18% | 20.68% | 6.23% | 2.60% | 33.33% | 3.44% | 6.51% | 2.61% | 19.18% |
| b. Total assembled size | 818 | 15,659 | 3,052 | 5,335 | 21,324 | 8,527 | 2,158 | 6,940 | 2,867 | 7,045 | 1,802 | 7,492 |
| of other repeats(3a X 1i) | | | | | | | | | | | | |
| 4 Non-repeat content | | | | | | | | | | | | |
| a. % of contigs with | 16.79% | 44.01% | 20.69% | 39.57% | 33.34% | 39.88% | 35.31% | 39.30% | 42.81% | 30.87% | 15.86% | 47.16% |
| unique sequence | | | | | | | | | | | | |
| (100 − (2a + 2c + 3a)) | | | | | | | | | | | | |
| b. Total assembled size | 14,303 | 42,306 | 27,215 | 17,332 | 34,378 | 54,583 | 29,304 | 8,183 | 35,676 | 33,407 | 10,950 | 18,421 |
| of non-repeat sequence | | | | | | | | | | | | |
| (4aX1i) | | | | | | | | | | | | |
| c. Number of contigs | 2 | 7 | 5 | 4 | 2 | 3 | 3 | 2 | 4 | 7 | 1 | 4 |
| with non-repeat | | | | | | | | | | | | |
| sequence >1 kb | | | | | | | | | | | | |

For each of the 11 MCs listed in Table 10, a listing of all sequences found in the assembled sequence for the MC with homology to the SCEN monomer (SEQ ID NO:73) was generated. In addition, the individual monomers were clustered based on their homology to the SCEN monomer consensus (SEQ ID NO:73), and the consensus sequence for each cluster was determined (data not shown). An overall consensus for the SCEN sequences in the particular MC was then generated and is provided below for each MC.

### (i) MC CHROM5802.

As described above, an overall consensus for the SCEN sequences in each particular MC was determined. For MC CHROM5802, the overall consensus was:

(136 bp; SEQ ID NO: 1)
CGTCACCTAGGAGTACCATCGGGTGCGTCCAAAATGATTTCTGAGCCTAT
GGTACGTTTGGCGCAAACCGTGCACCTATCTTGCGCCAACACTAACACTA
TCTCCAAACGGACCGAAACGAGATTCCACATGAGCC

In addition, for MC CHROM5802, 51 sequences within the assembled sequence for the MC with homology to the SCEN monomer (SEQ ID NO:73) were sequenced. The individual sequences ranged in length from 130 to 136 bp. The percent homology to the SCEN consensus sequence (SEQ ID NO:73) was determined and ranged from 87% to 91% homology. In addition, the percent homology of each of the 51 individual SCEN-like sequences to the overall consensus sequence identified for the specific MC (see above; SEQ ID NO:1) was determined, and ranged from 84% to 92%. A summary of the information generated for MC CHROM5802 is provided below in Table 11 for 5 exemplary contigs.

TABLE 11

| | | | Start | End | Length of | |
|---|---|---|---|---|---|---|
| MC CHROM5802 | Homology | Homology | position of | position | SCEN | |
| Contig Unique | to SEQ ID | to SEQ ID | SCEN | of SCEN | Repeat | |
| Name & Position | NO: 73 | NO: 1 | Repeat | Repeat | (bp) | MC-monomer Sequence |
| >contig00003_ | 91 | 91 | 1 | 136 | 136 | CGTCACCTAGGAGTTGCATCGGGTGCGTCCAAAAT |
| 215 − _30900_30765 | | | | | | GATTTTTGAGCCTATGGTACGTTCGGCGCAAACCG |
| | | | | | | TGCAACCATCTTGCATCAAGATTGACACTATCTCC |
| | | | | | | AAATGGACCAAAACGAGCTTCCACTTGAGCC |
| | | | | | | (SEQ ID NO: 13) |
| >contig00005_ | 89 | 88 | 1 | 131 | 131 | ACCTATGTGTTCCATCGGGTGTGTCCAACCGATTT |
| 332 − _4129_3999 | | | | | | CTAAGCCTATGGTACGTTTAGCGCAAACCATGCAC |
| | | | | | | CTATCTTGCGTCAAGACTGGCACTATCTCCAAACG |
| | | | | | | GACCAAAACGAGCTTCCACTTAAGCC (SEQ ID |
| | | | | | | NO: 14) |
| >contig00003_ | 88 | 92 | 1 | 134 | 134 | TCACCTAGGAGTACCATCGGGTGCGTCCAAAATGA |
| 260 − _40247_40114 | | | | | | TTACTAAGCCTATGGTACGTTTGGCACAAACCGTG |
| | | | | | | CACCTATCTTGAGTCAAGATTAACACTATCTGCAA |
| | | | | | | ACGGACCGAAATGAGCTTCCACTTGAGCC (SEQ |
| | | | | | | ID NO: 15) |
| >contig00003_ | 88 | 85 | 1 | 134 | 134 | GTCACCTAGGAGTTCCATCAGGTGCATCCAAAATA |
| 242 − _37796_37663 | | | | | | ATTTCCGAGCCTATGTATGTTCGGTGCAAACTGTG |
| | | | | | | CACCTATCTTGCGTCAAGATTGGCACTATCTCTAA |
| | | | | | | ACGGACCAAAACGAGCTTCCACTTGTGCC (SEQ |
| | | | | | | ID NO: 16) |
| >contig00003_ | 87 | 84 | 1 | 135 | 135 | GTCACCTAGCAGTTCCATCAGGTGTGTCCAAAACA |
| 161 − _23264_23130 | | | | | | ATTTCTGAGCCTATGGTACGTTCGGCGCAAACCAT |
| | | | | | | GCAAATATTTTGAGTCAAGATTAGCAATATCTCCA |
| | | | | | | AATGGACTAAAATGAGCTTCCACTTGAACC (SEQ |
| | | | | | | ID NO: 17) |

### (ii) MC CHROM5809.

For MC CHROM5809, the overall consensus for the SCEN sequences was:

(135 bp; SEQ ID NO: 2)
CGTCACCTAGGAGTACCATCGGGTGCGTCCAAAATGATTTCTGAGCCTAT
GGTACGTTCGGCGCAAACCATGCACCTATCTTGCATCAAGATTAGCACTA
TCTCCAAACGGACTGAAACGAGCTTCCACTTGACC

In addition, for MC CHROM5809, 247 sequences within the assembled sequence for the MC with homology to the SCEN monomer (SEQ ID NO:73) were sequenced. The individual sequences ranged in length from 105 to 139 bp. The percent homology to the SCEN consensus sequence (SEQ ID NO:73) was determined and ranged from 74% to 89% homology. In addition, the percent homology of each of the 247 individual SCEN-like sequences to the overall consensus sequence identified for the specific MC (see above; SEQ ID NO:2) was determined, and ranged from 74% to 100%. A summary of the information generated for MC CHROM5809 is provided below in Table 12 for 5 exemplary contigs.

TABLE 12

| | | | | | Length | |
|---|---|---|---|---|---|---|
| MC CHROM5809 | | | Start | End | of | |
| Contig Unique | Homology to | Homology to | position | position | SCEN | |
| Name | SEQ ID | SEQ ID | of SCEN | of SCEN | Repeat | |
| & Position | NO: 73 | NO: 2 | Repeat | Repeat | (bp) | MC-monomer Sequence |
| >contig00002_ | 81 | 85 | 1 | 128 | 131 | CCTAGGAGTTACAACGGGTGCGTCCAGAATGATTTCT |
| 2 − _673_543 | | | | | | GAGCTAGTGATACATTCTGCACAAAGCGTGCACCTAT |
| | | | | | | CTTGCAACAAGATTAACACTATCTGCAAACAGATTGA |
| | | | | | | ATCGAGCTTCCACTTGAGCC (SEQ ID NO: 18) |
| >contig00017_ | 77 | 100 | 2 | 9 | 108 | AGAAACGAGGGCAGGAGGTGAAGCGGAACGGAGGTGG |
| 53 + _3584_3691 | | | | | | CCACGGAGCGGTGTTCTTCGGTGAGCTCGACCGGGAC |
| | | | | | | TTCGTCGAGCGATTCCCACAAAGGGGAATCTTAC |
| | | | | | | (SEQ ID NO: 19) |
| >contig00035_ | 89 | 89 | 1 | 132 | 135 | GTCACCTAGGAGTTAAATCGGGTGCGTCCAAAACTAT |
| 172 − _3681_3547 | | | | | | TTATGAGCCTATGGTACGTTCGGCGCAAACCGTGCAC |
| | | | | | | CTATCTTGCATCAAGATTAGCTCAATCTCCAAACAGA |
| | | | | | | TTAAATCGCGCTTCCACTTGAGCC (SEQ ID |
| | | | | | | NO: 20) |
| >contig00045_ | 74 | 78 | 1 | 128 | 130 | TCACCTTAGATTACCGTCGGGTGTGTGCAAAACAATT |
| 183 − _314_185 | | | | | | TATGAGCCTATGGCACGTTTGGCACAAACCATTATCT |
| | | | | | | TGCACCGACACTAACACTGTCACCAAACGGAATGAAA |
| | | | | | | CGAGATTCCACATCACCCA (SEQ ID NO: 21) |
| >contig00052_ | 74 | 74 | 1 | 114 | 116 | GGGGGCATCAAGAACGATTTTGAGCCTCTGGTACGTT |
| 215 − _2261_2146 | | | | | | TGGCGCAAATCATGCACCTATCTTACATCGACAGTAA |
| | | | | | | CATTGTCTCCAAATAGTACAAAATGACACTCTACACG |
| | | | | | | ACCCA (SEQ ID NO: 22) |

(iii) MC CHROM5810.

For MC CHROM5810, the overall consensus for the SCEN sequences was:

(137 bp; SEQ ID NO: 3)
CGTCACCTAGGAGTTCCATCGGGTGCGTCCAAAATGATTTCTGAGCCTAT
GGTACGTTTGACGCAAACCGTGCACCTATCTTGCACCAACACTAACACTA
TCTCCAAACGGAAAGAAGCGAGATTCCACATGACCCA

In addition, for MC CHROM5810, 652 sequences within the assembled sequence for the MC with homology to the SCEN monomer (SEQ ID NO:73) were sequenced. The individual sequences ranged in length from 106 to 138 bp. The percent homology to the SCEN consensus sequence (SEQ ID NO:73) was determined and ranged from 70% to 91% homology. In addition, the percent homology of each of the 652 individual SCEN-like sequences to the overall consensus sequence identified for the specific MC (see above; SEQ ID NO:3) was determined, and ranged from 73% to 92%. A summary of the information generated for MC CHROM5810 is provided below in Table 13 for 5 exemplary contigs.

TABLE 13

| | | | | | Length | |
|---|---|---|---|---|---|---|
| MC CHROM5810 | Homology | Homology | Start | End | of | |
| Contig | to | to | position | position | SCEN | |
| Unique Name & | SEQ ID | SEQ ID | of SCEN | of SCEN | Repeat | |
| Position | NO: 73 | NO: 3 | Repeat | Repeat | (bp) | MC-monomer Sequence |
| >contig00003_ | 70 | 79 | 1 | 137 | 137 | CGTCACCTAGGAGTATAATCGGCTGTGTCATAAATGATTTTTTG |
| 29 + _2207_2343 | | | | | | AGCTGATGGCACGTTTGGTGCAAACTGTGCACCTATCTTGCACT |
| | | | | | | GAAACTAACAGTCTCCAAAAAGAAAAGAAGTGAGAATCAACATT |
| | | | | | | ACCCA (SEQ ID NO: 23) |
| >contig00003_ | 91 | 86 | 1 | 136 | 136 | CGTCACCTAGGAGTTCCATCGGGTGCGTCCAAAATGATTTCTAA |
| 54 + _5849_5984 | | | | | | GCCTATGGTACGTTCGACGCAAACCATGCACCTATCTTGCGTCA |
| | | | | | | AGATGAGCACTATCTCCGAAAGGACCGAAACAAGCTTCCACTTG |
| | | | | | | AGCC (SEQ ID NO: 24) |
| >contig00003_ | 80 | 85 | 1 | 112 | 131 | CGTCACCTAGGAGATCCATCGGGTGCATAAAAAATGATGTCCGA |
| 71 + _8229_8359 | | | | | | GCCTATGGTACTTTTGACACAAATCGTGCACCTATCTTGCATCA |
| | | | | | | AGATTTACACTATCTCTCAATGGACCGATCTTTCACTTGAGCC |
| | | | | | | (SEQ ID NO: 25) |
| >contig00003_ | 83 | 92 | 1 | 137 | 137 | CGTCACCTAGGAGTACCATCGGCTGCGTCCAAAATGATTTCTAA |
| 77 + _9131_9267 | | | | | | GCCGGTGGTACGTTTGACGCAAAGTGCGCACCTATCTTGCACCA |
| | | | | | | ACACTAACACTATCTCCAAACGGAAAGAAGTGAGCTTCCACATG |
| | | | | | | ACCCA (SEQ ID NO: 26) |
| >contig00019_ | 78 | 73 | 1 | 136 | 136 | CGTCACCTAGGAGTTCCATCGAGTGTGTCCAAAATGATTACCAG |
| 330 − _252_117 | | | | | | GATGATCAGACGTTCCGCGCCAACCATGCACCTACCTTGTGTTA |
| | | | | | | AAATTAGCGCTATCTCTAAAAGGACCAAAACAAGCTTCCACTTG |
| | | | | | | AGCC (SEQ ID NO: 27) |

### (iv) MC CHROM5814.

For MC CHROM5814, the overall consensus for the SCEN sequences was:

(137 bp; SEQ ID NO: 4)
CGTCACCTAGGAGTACCATCGGGTGCGTCCAAAACGATTTCTGAGCCTAT
GGTACGTTTGGCGCAAACCGTGCACCTATCTTGCACCAACACTAACACTA
TCTCCAAATGGACCGAAACGAGATTCCAGATGACCCA

In addition, for MC CHROM5814, 138 sequences within the assembled sequence for the MC with homology to the SCEN monomer (SEQ ID NO:73) were sequenced. The individual sequences ranged in length from 102 to 145 bp. The percent homology to the SCEN consensus sequence (SEQ ID NO:73) was determined and ranged from 73% to 89% homology. In addition, the percent homology of each of the 138 individual SCEN-like sequences to the overall consensus sequence identified for the specific MC (see above; SEQ ID NO:4) was determined, and ranged from 77% to 92%. A summary of the information generated for MC CHROM5814 is provided below in Table 14 for 5 exemplary contigs.

TABLE 14

| | | | | | Length | |
|---|---|---|---|---|---|---|
| MC CHROM5814 | Homology | Homology | Start | End | of | |
| Contig | to | to | position | position | SCEN | |
| Unique Name & | SEQ ID | SEQ ID | of SCEN | of SCEN | Repeat | |
| Position | NO: 73 | NO: 4 | Repeat | Repeat | (bp) | MC-monomer Sequence |
| >contig00009_ | 87 | 83 | 2 | 118 | 117 | GTCACCTAGGGGTTCCATCGGGAGCGTCCAAAATAATTTTTGAG |
| 6 + _739_855 | | | | | | CCTATGGTACGTTCGGAACAAACCGTGCACCTATCTTGCGTCAA |
| | | | | | | GATTAGCACCATCTCTAAATAGACCAAAA (SEQ ID NO: 28) |
| >contig00044_ | 78 | 77 | 2 | 95 | 116 | GTCGCCTAGGAGTTCCATCCGGAGCGTCCAAAACTATTTCTGAA |
| 23 + _2121_2236 | | | | | | CCAGTGGTACGATCTGTGCAAACCGTGCATCAAGATTAGCACTA |
| | | | | | | TCTCCAAATGGACCAAAGTTGAGCCTCG (SEQ ID NO: 29) |
| >contig00209_ | 86 | 92 | 67 | 123 | 134 | TGTGCATAATAACTGTTGGGGTCCGGGCAACCTTAATTTATTGG |
| 64 + _875_1008 | | | | | | GCCTCGGCCAACCCTACCTGGGCCGGATGTATCACCGTGCACCT |
| | | | | | | ATCTTGCACTAACACTAACATCATCTCCAAACAGACCGAAACGA |
| | | | | | | GA (SEQ ID NO: 30) |
| >contig00209_ | 73 | 81 | 1 | 137 | 137 | CGTGACCTAGGAGTACCATCGGGTGCGTCCAAAACGATTTCTGA |
| 88 + _4602_4738 | | | | | | TCCTTTGGTATGTTTGTTCACCAATCCTGCTCTGACACAAACAA |
| | | | | | | CGTCTCCAAACGGACCGAAACGAGATTCCACATGACCCACGTTC |
| | | | | | | GGTGG (SEQ ID NO: 31) |
| >contig00583_ | 89 | 84 | 16 | 126 | 112 | TCCATCAGGTGCGTCCAAAACTATTTGTAGGCCTATGGTACGTT |
| 135 + _91_202 | | | | | | CGGCGCAAACCATGCACCTATCTTGCGTCAAGATTAGCACTATT |
| | | | | | | TCTAAATGGACCAAAACGAGCTTC (SEQ ID NO: 32) |

### (v) MC CHROM5817.

For MC CHROM5817, the overall consensus for the SCEN sequences was:

(137 bp; SEQ ID NO: 5)
TCACCTAGGAGTACCATCGGGTGCGTCCAAAACGATTTCTGAGCCTATGG
TACGTTCGGCGCAAACCGTGCACCTATCTTGCACCAACACTAACACTATC
TCCAAACGGACCGAAACGAGTTTTCCAGATGACCCACG

In addition, for MC CHROM5817, 454 sequences within the assembled sequence for the MC with homology to the SCEN monomer (SEQ ID NO:73) were sequenced. The individual sequences ranged in length from 102 to 139 bp. The percent homology to the SCEN consensus sequence (SEQ ID NO:73) was determined and ranged from 73% to 91% homology. In addition, the percent homology of each of the 454 individual SCEN-like sequences to the overall consensus sequence identified for the specific MC (see above; SEQ ID NO:5) was determined, and ranged from 72% to 100%. A summary of the information generated for MC CHROM5817 is provided below in Table 15 for 5 exemplary contigs.

TABLE 15

| | | | | | Length | |
|---|---|---|---|---|---|---|
| MC CHROM5817 | Homology | Homology | Start | End | of | |
| Contig | to | to | position | position | SCEN | |
| Unique Name & | SEQ ID | SEQ ID | of SCEN | of SCEN | Repeat | |
| Position | NO: 73 | NO: 5 | Repeat | Repeat | (bp) | MC-monomer Sequence |
| >contig00015_ | 84 | 82 | 1 | 134 | 135 | GTCACCTAGGAGTTCAATTGGGTGCGTCAAAAATGATTTTCGAG |
| 26 − _2977_2843 | | | | | | CCTATGGTACGTTTGGCACAAACCATGCACCTATCTTGCATCAA |
| | | | | | | GGTTAGCACCATCTCCAAATGGACCGAAATGTACTTCCACATGA |
| | | | | | | GCC (SEQ ID NO: 33) |
| >contig00021_ | 73 | 81 | 3 | 135 | 132 | ACCTAGGACTAAAGTCGGGTGTGTCCAAAATGATTTTGAGCCTA |
| 106 −_28125_ | | | | | | TTGTATGTTTGGCGCAAGTCGTGCACATATCTTGCACCGACCCT |
| 27994 | | | | | | AACGCCGTCTCCAAACAAATCAAAACAAGATTCCATAGGACCCA |
| | | | | | | (SEQ ID NO: 34) |
| >contig00038_ | 85 | 100 | 3 | 10 | 135 | TCAATTAATCTCGTCTTGTACCAAGTTTTAACTAAAGAAACTCC |
| 244 − _10936_ | | | | | | CTTGATGATCTTTACTCCATAAGTTTCTAATTCTTGGGCAACTT |
| 10802 | | | | | | CTGGATGCACTTGGTTACACTGTCAACTCAAAACAAAAACCTAG |
| | | | | | | GAC (SEQ ID NO: 35) |
| >contig00049_ | 73 | 72 | 1 | 134 | 136 | CGTCTCCTAGGACTTCTATCGGGTGCATCCGAAATGATTTCTGA |
| 284 + _558_693 | | | | | | GTCGACAGTACATTCAGCGATGTGCGACGACCTTGTGTTAAGAT |
| | | | | | | TAACACTATCTCTAAATGAACTGAAATGAGCTTCTAGTTGAGCC |
| | | | | | | CTAT (SEQ ID NO: 36) |
| >contig00049_ | 91 | 86 | 1 | 127 | 128 | CGTCATCTAGGAGTTCCATCGGGTGAGTCCAAAACTATTTCTAA |
| 302 + _3643_3770 | | | | | | GCCTATGGTACGTTCGACGCAAACCATGCACCTATCTTGCATCA |
| | | | | | | AGATTAGCACTATCTCCATTGGACCAAAATGAGCTTCCAG |
| | | | | | | (SEQ ID NO: 37) |

### (vi) MC CHROM5819.

For MC CHROM5819, the overall consensus for the SCEN sequences was:

(135 bp; SEQ ID NO: 6)
CCGTCACCTAGGAGTTCCATCGGGTGCGTCCAAAATGATTTCTGAGCCTA
TGGTACGTTTGGCGCAAACCGTGCACCTATCTTGCGTCAAGATTAGCACT
ATCTCCAAACGGACTGAATCGAGCTTCCACTTGAG

In addition, for MC CHROM5819, 551 sequences within the assembled sequence for the MC with homology to the SCEN monomer (SEQ ID NO:73) were sequenced. The individual sequences ranged in length from 103 to 147 bp. The percent homology to the SCEN consensus sequence (SEQ ID NO:73) was determined and ranged from 73% to 89% homology. In addition, the percent homology of each of the 551 individual SCEN-like sequences to the overall consensus sequence identified for the specific MC (see above; SEQ ID NO:6) was determined, and ranged from 74% to 100%. A summary of the information generated for MC CHROM5819 is provided below in Table 16 for 5 exemplary contigs.

TABLE 16

| | | | | | Length | |
|---|---|---|---|---|---|---|
| MC CHROM5819 | Homology | Homology | Start | End | of | |
| Contig | to | to | position | Position | SCEN | |
| Unique Name & | SEQ ID | SEQ ID | of SCEN | of SCEN | Repeat | |
| Position | NO: 73 | NO: 6 | Repeat | Repeat | (bp) | MC-monomer Sequence |
| >contig00003_ | 83 | 87 | 1 | 134 | 136 | CGTCACCAAGGAGTTCCATTGGGTGTGTTCAAAACAATTTCTGA |
| 9 + _116_251 | | | | | | TCCTAGGGTACGTTTGGCACAAACTGCGCACCTATCTTGTATCA |
| | | | | | | AGATTAGCCCTATCTGCAAACAGACTGAATCAAGCTTCCACTTG |
| | | | | | | AGC (SEQ ID NO: 38) |
| >contig00004_ | 76 | 74 | 1 | 134 | 138 | CGTCACCTAGGAGTTCAATGATGTGTGTCCAAAACGATTTTCTA |
| 20 + _251_388 | | | | | | AGCCTATGGTTTGTTCAGCCCAAACCAAGCACCTACTGTGCACC |
| | | | | | | GACACAAATAATATCTCCAAACAGACCAAAATGAGATTCCACAT |
| | | | | | | GATCCA (SEQ ID NO: 39) |
| >contig00013_ | 85 | 100 | 101 | 95 | 133 | TAGGAGGCTTGTAATGCTCCGGGGCTTTTGCCTAGGTTTGAAGC |
| 205 − _10264_10132 | | | | | | ACAATGTCAGTTAGCTTCATCTTCAGGACATGATCAACCATCAT |
| | | | | | | CTTTATCTCCTAGACTTGGTCACTGTTACACCATCTTGTGATCG |
| | | | | | | G (SEQ ID NO: 40) |
| >contig00014_ | 73 | 78 | 1 | 119 | 121 | CGTCACCTAGGAGTACCATCAGGTGTGTCCAAAATGGTTTCTGA |
| 336 + _8811_8931 | | | | | | GTCTATGGTATGTTTGGCACAAACCGTCAAGATTAGCATTATCT |
| | | | | | | CCTAACGGAATGAAATGAGCATGCACTTGAGCC (SEQ ID |
| | | | | | | NO: 41) |
| >contig00020_ | 89 | 90 | 1 | 113 | 113 | GTCACCTAGGAGTTCCATCGAGTGTGTCCGAAACGATTTCCGAG |
| 481 + _9997_10109 | | | | | | CCTTTTGGTATGTTCGGCGCAAAACGTGCACCTATCTTGCATCA |
| | | | | | | AGATTAGCACTATCTCCAAACGGAC (SEQ ID NO: 42) |

(vii) MC CHROM5820.

For MC CHROM5820, the overall consensus for the SCEN sequences was:

(136 bp; SEQ ID NO: 7)
TCACCTAGGAGTTCCATCGGGTGCGTCCAAAACTATTTATGAGCCTATGG
TACGTTCGATGCAAACCGTGCACCTATCTTGCATCAAGACTAGCACTATC
TCCAAACGGACCGAACCGAGATTCCACATGACCCCG

In addition, for MC CHROM5820, 351 sequences within the assembled sequence for the MC with homology to the SCEN monomer (SEQ ID NO:73) were sequenced. The individual sequences ranged in length from 103 to 138 bp. The percent homology to the SCEN consensus sequence (SEQ ID NO:73) was determined and ranged from 73% to 92% homology. In addition, the percent homology of each of the 351 individual SCEN-like sequences to the overall consensus sequence identified for the specific MC (see above; SEQ ID NO:7) was determined, and ranged from 75% to 100%. A summary of the information generated for MC CHROM5820 is provided below in Table 17 for 5 exemplary contigs.

TABLE 17

| | | | | | Length | |
|---|---|---|---|---|---|---|
| MC CHROM5820 | Homology | Homology | Start | End | of | |
| Contig | to | to | position | position | SCEN | |
| Unique Name & | SEQ ID | SEQ ID | of SCEN | of SCEN | Repeat | |
| Position | NO: 73 | NO: 7 | Repeat | Repeat | (bp) | MC-monomer Sequence |
| >contig00011_ | 73 | 76 | 1 | 134 | 131 | CGTCACCTTGGAGTACCATTTGGTTTGTGTAAAACGATTTATGA |
| 52 − _956_826 | | | | | | GCTCATGGCACATTTGACGCAAACCAAGCACCACTTGTCAAGAT |
| | | | | | | TAGCACTATCGGCAAACACACCGAACAGATCTCCACCAGACCC |
| | | | | | | (SEQ ID NO: 43) |
| >contig00014_ | 92 | 90 | 1 | 134 | 134 | TCACCTAGGAGTTCCATCGGGTGCCTCCAATACTATTTCTGAGC |
| 128 − _698_565 | | | | | | CTACGGTACGTTCGATGCAAACCGTGCACCAATCTTGCATCAAG |
| | | | | | | AGTAGCACTATCTCCAAACGGACCAAAGAGAGCTCCCACTTGAG |
| | | | | | | CC (SEQ ID NO: 44) |
| >contig00016_ | 74 | 75 | 6 | 134 | 124 | TAGGAGTATCATGGGTGCGTCTAAAATGATTTATGAGCCTATGG |
| 172 − _1713_1590 | | | | | | TACGTTTGACACAAACCAAGCATTTGCACCGACGCTAACACTGT |
| | | | | | | CTCAATGTAGATCAAAACAAGATTCCACATGACCCA (SEQ ID |
| | | | | | | NO: 45) |
| >contig00024_ | 80 | 100 | 99 | 105 | 130 | CCTTTTCTATCAACGGTGCTGCGTCACCTTATTTTGGGCCGATG |
| 241 − _9859_9730 | | | | | | GCCCATGTATCAAGTTGGGTCCATTAGGGACGCGTCCTAGGGTT |
| | | | | | | GGAGAATGACTCAAACACCCTTGTGGTCGTCCTCCCATCTAC |
| | | | | | | (SEQ ID NO: 46) |
| >contig00036_ | 87 | 86 | 1 | 115 | 117 | CGTCACCTAGGAGTTCCATCGGGTGCCGCTAATACTATTTCTGA |
| 293 − _118_2 | | | | | | GCCAACGGTACGTTCGACGCAAATCGTGCACCAATCTTGCACCA |
| | | | | | | AGTGTAGCACTGTCTCCAAACGGATCGAA (SEQ ID NO: 47) |

(viii) MC CHROM5823.

For MC CHROM5823, the overall consensus for the SCEN sequences was:

(135 bp; SEQ ID NO: 8)
CGTCACCTAGGAGTWCCATCGGGTGCGTCCAAATTGATTTCYGAGCCTAT
GGTACGTTCGGCGCAAACCGTGCAACTATCTTGCAYCRACACTAACACTA
TCTCCAAACAGACCGAAACAAGATTCCACWTGASM

In addition, for MC CHROM5823, 46 sequences within the assembled sequence for the MC with homology to the SCEN monomer (SEQ ID NO:73) were sequenced. The individual sequences ranged in length from 103 to 136 bp. The percent homology to the SCEN consensus sequence (SEQ ID NO:73) was determined and ranged from 72% to 88% homology. In addition, the percent homology of each of the 46 individual SCEN-like sequences to the overall consensus sequence identified for the specific MC (see above; SEQ ID NO:8) was determined, and ranged from 75% to 88%. A summary of the information generated for MC CHROM5823 is provided below in Table 18 for 5 exemplary contigs.

TABLE 18

| | | | | | Length | |
|---|---|---|---|---|---|---|
| MC CHROM5823 | Homology | Homology | Start | End | of | |
| Contig | to | to | position | position | SCEN | |
| Unique Name & | SEQ ID | SEQ ID | of SCEN | of SCEN | Repeat | |
| Position | NO: 73 | NO: 8 | Repeat | Repeat | (bp) | MC-monomer Sequence |
| >contig00024_ | 72 | 79 | 1 | 133 | 133 | CGTCTCCTAGGAGTACCATCAAGTGCGTCCAAATCGATTTCAGG |
| 5 - _224_92 | | | | | | GTGTGTTACCTGATTGGTACGAACCGTCCAACTATCTTGCACCG |
| | | | | | | ACACTAACACTGTCTCCAAATAGACTGAAACAAGATTCCACATG |
| | | | | | | A (SEQ ID NO: 48) |
| >contg00045_ | 84 | 75 | 17 | 133 | 114 | CATCGGATGCATCTAAACCAATTTCCGAGCCTATGGTATGTTCT |
| 16 − _377_264 | | | | | | GCGCAACCCGTGCACCTCGTCAAGATCAGCACTATCTCCAAACG |
| | | | | | | GACCAAAACGAGCTTCCACTTGAGCC (SEQ ID NO: 49) |
| >contg00088_ | 80 | 88 | 1 | 133 | 133 | CGTCACCTAGGAGTACCATCGGGTGCGTCCAAATTGATTTCAGG |
| 26 + _810_942 | | | | | | GCGTATGGTACGATTGGTGCGAACCGTCCAACTATCTTGCACCG |
| | | | | | | ACACTAACACTATCTCCAAATAGACTGAAACAAGATTCCGCATG |
| | | | | | | A (SEQ ID NO: 50) |
| >contig00236_ | 88 | 81 | 1 | 133 | 136 | CGACACCTAGGAGTTCCATTGGGTGCGTCCAAAATGATTCTCGA |
| 36 − _193_58 | | | | | | GCCTCTGGTACGTTCTGCGCAAACCGTGCACCTATCTTGCAGAA |
| | | | | | | AGATTAGCACCATCTCCAAATGGAACAAAACGAGCTTCCACTTG |
| | | | | | | AGCC (SEQ ID NO: 51) |
| >contig00311_ | 84 | 78 | 16 | 133 | 120 | CCATGGGGTGTGTCCAAAATGATTTCCAATCCTATGGTATGTTT |
| 46 + _1_120 | | | | | | TGCGTAATCCGTGCACCTATCTTGCATCAAGTTTAGCACCATCT |
| | | | | | | CCAATCGGACCGTAACGAGCTTCCACTTGAGC (SEQ ID |
| | | | | | | NO: 52) |

### (ix) MC CHROM5829.

For MC CHROM5829, the overall consensus for the SCEN sequences was:

(133 bp; SEQ ID NO: 9)
CGTCACCTAGGAGTACCATCGGGTGTGCCCAAAATGATTTCCAAGCCTAT
GGTACATTCGGCGCAAACCGTGCACCTATCTTGCGTCGAGATTAACACTA
TCTCCAAACGGACCGAAACGAGCTTCCACTTGA

In addition, for MC CHROM5829, 282 sequences within the assembled sequence for the MC with homology to the SCEN monomer (SEQ ID NO:73) were sequenced. The individual sequences ranged in length from 105 to 149 bp. The percent homology to the SCEN consensus sequence (SEQ ID NO:73) was determined and ranged from 70% to 90% homology. In addition, the percent homology of each of the 282 individual SCEN-like sequences to the overall consensus sequence identified for the specific MC (see above; SEQ ID NO:9) was determined, and ranged from 74% to 93%. A summary of the information generated for MC CHROM5829 is provided below in Table 19 for 5 exemplary contigs.

TABLE 19

| | | | | | Length | |
|---|---|---|---|---|---|---|
| MC CHROM5829 | Homology | Homology | Start | End | of | |
| Contig | to | to | position | position | SCEN | |
| Unique Name & | SEQ ID | SEQ ID | of SCEN | of SCEN | Repeat | |
| Position | NO: 73 | NO: 9 | Repeat | Repeat | (bp) | MC-monomers equence |
| >contig00001_ | 83 | 87 | 3 | 133 | 130 | TCACCTAGGAGTACCATCGGGTGCATACAAAATAATTTCCAAGC |
| 10 − _1699_1570 | | | | | | CTATGGTACCTTCGGCGCAAACCATGCATTATCTTGCGCTAAGA |
| | | | | | | CTAACACTATCTCTAAACAGACCGAAACAAGCTTTCACTTGA |
| | | | | | | (SEQ ID NO: 53) |
| >contig00001_ | 82 | 93 | 1 | 133 | 136 | CGTCACCTAGGAGTACCATCGGCTGTGCCTAAAATGATTTCCAC |
| 24 − _3611_3476 | | | | | | GCCCATGGTACATTCGGCCCAAACCGTGCACCTATCTTGCGGCG |
| | | | | | | AGATTAACACTATCTCCAAATGGACCGAAACGAGTTCCACTTGA |
| | 70 | 78 | 24 | 133 | 113 | CCCA (SEQ ID NO: 54) |
| >contig00001_ | | | | | | AGCTCCAGGGCATGTGCGGAAAGTGATTTCCATGCCCGTGGTAG |
| 47 − _6711_6599 | | | | | | ATTTGTCGCAAACCGTGCACCTATCTTGTGATTCTCCAAACGGA |
| | | | | | | GCGAAATGAGCTTCCACTTGACCCA (SEQ ID NO: 55) |
| >contig00007_ | 90 | 92 | 1 | 133 | 133 | CGTCACCTAGGAGTACCATCGGGTGCGTCCAAAATTATTTCTAA |
| 198 − _6892_6760 | | | | | | GCCTATGGTACGTTTGGCGCAAACCGTGCACCTATCTTGCGCCG |
| | | | | | | AGACTAACACTATCTCCAAACGGACCCAAACAAGCTTCCACTTG |
| | | | | | | A (SEQ ID NO: 56) |
| >contig00007_ | 73 | 74 | 1 | 133 | 121 | CGTCACCTAGGAGTACCATTAGGTGCATTCAAAATAATTTCCAA |
| 252 − _42859_ | | | | | | GCTTATGGTACGTTACCTATCTTGCACCAAGGCTAACACTATCT |
| 42739 | | | | | | CTAAACGGATCGAAATGAGATTCCCTTGACCCA (SEQ ID |
| | | | | | | NO: 57) |

### (x) MC CHROM5834.

For MC CHROM5834, the overall consensus for the SCEN sequences was:

(136 bp; SEQ ID NO: 10)
GTCACCTAGGAGTTCCATCGGGTGCGCCCAAAATGATTTCCGAGCCTATG
GTACGTTCGGCGCAAACCGTGCACCTATCTTGCGTCGAGATTAACACTAT
CTCCAAACAGACCGAAACGAGCTTCCACTTGACCCA

In addition, for MC CHROM5834, 482 sequences within the assembled sequence for the MC with homology to the SCEN monomer (SEQ ID NO:73) were sequenced. The individual sequences ranged in length from 102 to 141 bp. The percent homology to the SCEN consensus sequence (SEQ ID NO:73) was determined and ranged from 70% to 85% homology. In addition, the percent homology of each of the 482 individual SCEN-like sequences to the overall consensus sequence identified for the specific MC (see above; SEQ ID NO:10) was determined, and ranged from 75% to 100%. A summary of the information generated for MC CHROM5834 is provided below in Table 20 for 5 exemplary contigs.

TABLE 20

| | | | | | Length | |
|---|---|---|---|---|---|---|
| MC CHROM5834 | Homology | Homology | Start | End | of | |
| Conig | to | to | position | position | SCEN | |
| Unique Name & | SEQ ID | SEQ ID | of SCEN | of SCEN | Repeat | |
| Position | NO: 73 | NO: 10 | Repeat | Repeat | (bp) | MC-monomer Sequence |
| >contig00003_ | 84 | 88 | 28 | 136 | 111 | GTCCATAACGATTTCTGAGCCTATGGTCTGTTTGACGCAAACCG |
| 8 + _1104_1214 | | | | | | TGCACCTATCTTGCGTCGAGATTAACACTATCTCCAAATAGACT |
| | | | | | | GAAACAAACTTCCATTTGACCCA (SEQ ID NO: 58) |
| >contig00003_ | 85 | 90 | 2 | 136 | 134 | TCACCTAGGAGTTCCATCGGGTGTGCCCAAAATGATTCCAAGCC |
| 73 + _10655_ | | | | | | TATTGTACATTCGACGCAAACCGTGCACCTATCTTGCATGTCGA |
| 10788 | | | | | | TTAACACTATTTCCAAACAGACCAAAACGAGCTTCCATTTGACC |
| | | | | | | CA (SEQ ID NO: 59) |
| >contig00004_ | 71 | 75 | 2 | 136 | 128 | TCACCTAGGAGTTCCATTGGGTGTGCCCAAAATGATTTTCAAGC |
| 231 − _4404_ | | | | | | CTATTAGACATTCAATGTGTGAATGGTGCACCTATCGTGTGTAA |
| 4277 | | | | | | CACTATCTCCAATGGCCGATATGAGCATCTACTTGACCCA |
| | | | | | | (SEQ ID NO: 60) |
| >contig00011_ | 80 | 100 | 9 | 15 | 136 | TATTTCTAAGTTCTAAGTTCTTATTTAGTTTTTACTTTATTCTA |
| 244 + _8700_ | | | | | | TTTACTTGAACTCCGCTTGTATTTGAGTGCTCTAGTTATAATAT |
| 8835 | | | | | | TTGGCTAGAGTAGTAAACCCTAGCGTAGATGTGGTGTCTAGGCT |
| | | | | | | ATAG (SEQ ID NO: 61) |
| >contig00027_ | 70 | 76 | 3 | 136 | 137 | CGCCACCTACGAGTTCCATCAGATGTGCCCAAAATGATTTCCAA |
| 427 − _13471_ | | | | | | GCTCGTTACACAATCAACACAATTTGGGCACCTATCATGTATCG |
| 13335 | | | | | | AGATTAGCACTACCACAATAGAGACTGAAACAAGCTTCCACTTA |
| | | | | | | ACCCA (SEQ ID NO: 62) |

### (xi) MC CHROM5839.

For MC CHROM5839, the overall consensus for the SCEN sequences was:

(136 bp; SEQ ID NO: 11)
CGTCACCTAGGAGTTCCATCGGGTGCGTCCAAAATGATTTCTGAGCCTAT
GGTACGTTTGGCGCAAACCGTGCACCTATCTTGCACCAAGACTAACACTA
TCTCCAAACGGACCGAAACGAGATTCCACATGACCC

In addition, for MC CHROM5839, 418 sequences within the assembled sequence for the MC with homology to the SCEN monomer (SEQ ID NO:73) were sequenced. The individual sequences ranged in length from 103 to 143 bp. The percent homology to the SCEN consensus sequence (SEQ ID NO:73) was determined and ranged from 71% to 90% homology. In addition, the percent homology of each of the 418 individual SCEN-like sequences to the overall consensus sequence identified for the specific MC (see above; SEQ ID NO:11) was determined, and ranged from 76% to 93%. A summary of the information generated for MC CHROM5839 is provided below in Table 21 for 5 exemplary contigs.

TABLE 21

| | | | | | Length | |
|---|---|---|---|---|---|---|
| MC CHROM5839 | Homology | Homology | Start | End | of | |
| Contig | to | to | position | Position | SCEN | |
| Unique Name & | SEQ ID | SEQ ID | of SCEN | of SCEN | Repeat | |
| Position | NO: 73 | NO: 11 | Repeat | Repeat | (bp) | MC-monomer Sequence |
| >contig00004_ | 89 | 88 | 1 | 136 | 137 | CGTCACCTAGAAGTTCCATCGGGTGCGTCCAAAATGATTTCTGA |
| 6 − _156_20 | | | | | | GCCCGTGGTAAGTTCGGCGCAAACCGTGCACCAATCTTGCGTCA |
| | | | | | | AGATTAGCACTATCTCCAAACAGACCGAATCGAACTTCCACTTG |
| | | | | | | AGCCA (SEQ ID NO: 63) |
| >contig00006_ | 77 | 76 | 1 | 132 | 135 | CGACACATAGGTATTCTACCGAGTGTGTCTAAAACAATTTTCGA |
| 52 −_5773_5639 | | | | | | GCATGTGGTATGTTCGGTGCATATCGTGCACCTTCCTGTGTCAA |
| | | | | | | GATTAGCACTATCTCCAAACGGACCGAAATGAGCATCCACTTGT |
| | | | | | | GCC (SEQ ID NO: 64) |
| >contig00013_ | 71 | 77 | 1 | 107 | 107 | CGTCATCCAGAACTATCAATGGCCGTGTCCAAAATGATTTCTAA |
| 324 −_305_199 | | | | | | GCCCTTGGTATGTTTGGCGCAAACGATGCACCTATCCTACACCG |
| | | | | | | ACACTAACACTAACACTAA (SEQ ID NO: 65) |
| >contig00018_ | 85 | 93 | 2 | 136 | 135 | GTCACCTAGGAGTACCATCGGGTGCGTCCAGAATGATTTCTGAG |
| 353 + _980_1114 | | | | | | CCTATGGTACATTTGGAGCAACCCATGCACCTATCTTGCACCGA |
| | | | | | | CACTAACACTATCTCCAAACGGACCGAAACGAGATTCCAAATGA |
| | | | | | | CCC (SEQ ID NO: 66) |
| >contig00020_ | 90 | 90 | 1 | 117 | 117 | CGTCACCTAGGAGTTCCATCGGGTGCATCCAAAATGATTTCCGA |
| 410 − _8366_8250 | | | | | | GCCTATGGTACGTTCGACGCAAACCGTGCACCTAGCTTGTGTCA |
| | | | | | | AGATGAGCACTATCTCCAAACGGACCGAA (SEQ ID NO: 67) |

(xii) MC CHROM5824.

For MC CHROM5824, the overall consensus for the SCEN sequences was:

(133 bp; SEQ ID NO: 12)
TGCGTCCAAAACGATTTCTGAGCCTATGGTACGTTTGGTGCAAACCGTGC
ACCTATCTTGTGTCAAGAGTAGCACTATCTCCAAACGGACCGAAAGAGCT
TCCACAAGCCCACGTCACCTAGGGTTCCATCGG

In addition, for MC CHROM5824, 166 sequences within the assembled sequence for the MC with homology to the SCEN monomer (SEQ ID NO:73) were sequenced. The individual sequences ranged in length from 100 to 138 bp. The percent homology to the SCEN consensus sequence (SEQ ID NO:73) was determined and ranged from 78% to 90% homology. In addition, the percent homology of each of the 166 individual SCEN-like sequences to the overall consensus sequence identified for the specific MC (see above; SEQ ID NO:12) was determined, and ranged from 80% to 90%. A summary of the information generated for MC CHROM5824 is provided below in Table 22 for 5 exemplary contigs.

TABLE 22

| | | | | | Length | |
|---|---|---|---|---|---|---|
| MC CHROM5824 | Homology | Homology | Start | End | of | |
| Contig | to | to | position | position | SCEN | |
| Unique Name & | SEQ ID | SEQ ID | of SCEN | of SCEN | Repeat | |
| Position | NO: 73 | NO: 12 | Repeat | Repeat | (bp) | MC-monomer Sequence |
| contig00005- | 83 | 88 | 1 | 136 | 136 | TGCGTCCCAAACTACTTGTGAGCCTATGGTACGTTCAGTGCAAA |
| 6-141 | | | | | | CCGTGCACCTATCTTGTGTCAAGATTGGCACTATCTCCAAACGG |
| | | | | | | ACCAAACAGAGCTCCACCAGACCCTCGTCACTTAGGAGTACCAT |
| | | | | | | CGGG (SEQ ID NO: 68) |
| contig00027- | 86 | 82 | 1 | 135 | 135 | TGCCTCCAATACTATTTCCGAGCATACGGTACATTTGACGCAAA |
| 275-409 | | | | | | CCGTGCACCAATCGTGCATCAAGAGTAGCACTATCTCCAAACGG |
| | | | | | | ACCGAACCAAGCTTCCACTTGAGCCTCGTCACCAAGGAGTCCCA |
| | | | | | | TCG (SEQ ID NO: 69) |
| contig00008- | 86 | 83 | 1 | 135 | 135 | TGCCTCCAATACTATTTCTGAGCCTATGGTATGTTTGACGCAAA |
| 1023-889 | | | | | | CCGTGCACCAATCGTGCATCAAGAGTAGCACTATCTCCAAACAG |
| | | | | | | ACCGAACCAAGCTTCCACTTGAGCCTCGTCACCAAGGAATCCCA |
| | | | | | | TCG (SEQ ID NO: 70) |
| contig00031- | 85 | 90 | 1 | 136 | 136 | TGCGTCAAAAACTATTTGTGAGCCTATGGTACGTCTAGTGCAAA |
| 305-170 | | | | | | CCGTGCACCTATCTTGTGTCAAGATTAGCACTATCTCCAAACGG |
| | | | | | | ACCGAACAGAGCTCCACCAGACCCACGTCACTTAGGAGTACCAT |
| | | | | | | CGGG (SEQ ID NO: 71) |
| contig00021- | 78 | 78 | 1 | 134 | 134 | TGCGTCTAAATTGATTTATGACCCTATGGTACGTTTGACGCAAA |
| 67-200 | | | | | | TTGAGCACCTATCATGCACCGACGCTAACACTGTCTCAAAATAG |
| | | | | | | ATCGAAACGAGATCCCACAAGACCCACGTCAACTAGGAGTTCCA |
| | | | | | | TC (SEQ ID NO: 72) |

### Example 6

### Testing of Sugar Cane MCs with a 5 Gene Stack

A subset of BAC clones identified in Example 3 and 4 were modified with a 5 gene stack, using the same procedure as described in Example 3, from donor plasmid CHROM5996, which contains genetic elements set out in Table 23.

TABLE 23
Donor Components of CHROM5996

| Genetic Element | Size (bp) | Location (bp) | Details |
|---|---|---|---|
| YAT1 yeast | 2000 | 7110-9109 | PCR amplified YAT1 promoter from chromosome I of |
| promoter | | | Saccharomyces cerevisiae for expression of NptII in sugar |
| | | | cane |
| Arabidopsis | 360 | 9123-9482 | PCR amplified Arabidopsis thaliana intron from UBQ10 gene |
| UBQ10 Intron | | | (At4g05320) for stabilization of NptII gene transcript and |
| | | | increase protein expression level |
| NPTII | 795 | 9510-10304 | Neomycin phosphotransferase II plant selectable marker |
| Rps16A | 489 | 10368-10856 | Amplified from Arabidopsis thaliana 40S ribosomal protein |
| terminator | | | S16 (At2g09990) for termination of NptII gene |
| ZmUbi1 | 1993 | 10898-12890 | PCR amplified Zea mays promoter and intron from |
| promoter + intron | | | polyubiquitin-1 (Ubi1 gene) (S94464.1) for expression of |
| | | | AmCyan fluorescent protein gene |
| AmCyan | 690 | 12911-13600 | Anemonia majano fluorescent protein gene (AF168421.1) |
| | | | encoding AmCyan fluorescent protein plant screenable |
| | | | marker |
| Terminator 6 | 332 | 13647-13978 | Terminator 6 from A. thaliana |
| Bacterial | 817 | 14159-14978 | Bacterial kanamycin selectable marker |
| kanamycin | | | |
| Nos terminator | 253 | 15075-15327 | Nopaline synthase terminator and PolyA addition site |
| GUS | 1809 | 15398-17206 | E. coli uidA gene encoding beta-glucuronidase plant screenable |
| | | | marker |
| RUBQ2 intron | 962 | 17285-18246 | PCR amplified Orysa sativa (rice) ubiquitin 2 gene intron for |
| | | | enhanced expression and stabilization of GUS transcript |
| RUBQ2 promoter | 1834 | 17285-18246 | PCR amplified Orysa sativa (rice) ubiquitin 2 gene promoter |
| | | | for expression GUS transcript |
| Terminator 6 | 332 | 20129-20460 | Terminator 6 from A. thaliana |
| AcGFP (nuc) | 831 | 20595-21425 | Nuclear localized AcGFP1 (Aequorea coerulescens GFP) |
| | | | green fluorescent protein gene encoding plant screenable |
| | | | marker |
| ZmUbi1 | 1993 | 21459-23451 | PCR amplified Zea mays promoter and intron from |
| promoter + intron | | | polyubiquitin-1 (Ubi1 gene) (S94464.1) for expression of |
| | | | AmCyan fluorescent protein gene |
| Nos terminator | 253 | 24676-23728 | Nopaline synthase terminator and PolyA addition site |
| PMI | | 23792-24967 | E. coli manA gene encoding phosphomannose isomerase |
| | | | (M15380.1) plant selectable marker |
| CMP | | 24979-25378 | Cestrum Yellow Leaf Curling Virus promoter (AF364175.3) |
| | | | for plant expression of PMI selectable marker gene |
| LoxP | 34 | 7057-7090 and | LoxP recombination sites for Cre mediated recombination |
| | | 25424-25457 | (Arenski et al 1983, Abremski et al 1984) |

The subset of BAC clones was selected from the total set of 51 BAC clones based on the test results described in Example 4 and/or sequence information described in Example 5. The resulting sugar cane MCs were provided with new clone numbers, and the first two columns of table 23 shows the cross referencing between the clone numbers of the MCs containing the two gene stack (described in Examples 3 and 4) and the MCs containing the 5 gene stack of this example. Sugar cane variety L97-128 was transformed in this set of experiments. Transformation and selection conditions were identical to those described in Example 4, and from these experiments a total of 2530 G418 resistant events were selected and analyzed by several molecular assays. Screening results from this experiment are shown in Table 24.

Out of the 2530 events selected on G418, 2887 (or 89.7%) were confirmed as true positives by PCR screening for the NPTII selectable marker gene as described in Example 4. Real time (RT-PCR) screening for two amplicons on the construct, the Yat1 promoter in the gene stack or the redF gene on the bacterial vector backbone, was done to determine the copy number of the MC construct in each NPTII positive transgenic line. Copy numbers were determined using an MJ Research PTC-200 qPCR machine, running the MJ Opticon Monitor Analysis software, by comparing the delta-Ct values of the Yat1 probe and the redF probe, to an internal control gene Adh1. Each PCR reaction consisted of 18.9 μl water, 2.5 μl 10×NEB Taq polymerase buffer, 1 μl 25 mM MgCl₂, 0.5 μl 40 μM dNTPs, 0.25 μl 20 μM Adh1 Forward primer, 0.25 μl 20 μM Adh1 Reverse primer, 0.25 μl 20 μM Yat1 or RedF Forward primer, 0.25 μl 20 μM Yat1 or RedF Reverse primer, 0.3 μl 20 μM Adh1 probe, 0.3 μl 20 μM Yat1 or RedF probe, and 0.5 μl NEB Taq Polymerase. The primer and probe sequences are as follow:

Adh1 Forward Primer (ADH1F):
(SEQ ID NO: 102)
5′-CCAGCCTCATGGCCAAAG-3′
Adh1 Reverse Primer (ADH1R):
(SEQ ID NO: 103)
5′-CCTTCTTGGCGGCTTATCTG-3
Yat1 Forward Primer (CHSL607):
(SEQ ID NO: 104)
5′-AAAGCATATAGCACAACA-3′
Yat1 Reverse Primer (CHSL608):
(SEQ ID NO: 105)
5′-TATGTTATGTGTGTCTTGTATA-3′
RedF Forward Primer (CHSL601):
(SEQ ID NO: 106)
5′-CATAAGTCCATCAGTTCAA-3′
RedF Reverse Primer (CHSL602):
(SEQ ID NO: 107)
5′-CCAAGGCATTTATTCTCA-3′
Adh1 Probe (ADH P):
(SEQ ID NO: 108)
5′-CTTAGGGGCAGACTCCCGTGTTCCCT-3′
Yat1 Probe(CHSL609):
(SEQ ID NO: 109)
5′-AGCATCAGCATCAGCACTAGA-3′
RedF Probe (CHSL603):
(SEQ ID NO: 110)
5′-TCGCAACCGCATCAGACT-3′

The Adh1 Probe was labeled with FAM, and the Yat1 or RedF probes were labeled with HEX. Copy number determinations with Yat1 and RedF were done in separate reactions in parallel. PCR cycling conditions were as follows, Step 1: 95° C. for 3 minutes, Step 2: 95° C. for 15 seconds, Step 3: 51° C. for 48 seconds (decreasing by 1 second per cycle), with Step 2 and 3 repeated for 35 cycles. Following the completion of the PCR reactions, results were analyzed with the MJ Opticon Monitor Analysis software. The copy number is calculated by comparing the delta Ct values in the experimental samples to the following controls used in each run: sugarcane genomic DNA from L97-128 untransformed callus; sugarcane gDNA spiked with a serial dilution of a plasmid containing the same copy number of amplicons for Adh1, Yat1 or RedF; and sugarcane gDNA spiked with a serial dilution of a mini-chromosome construct containing equal copies of Yat1 and RedF. Only transgenic events with a low copy number (between 0.5 and 3 copies) were selected for further analysis by fluorescent is situ hybridization (FISH) as described in Example 4. A total of 626 (or 21.7% of all PCR positive) transgenic lines were identified as low copy events. Out of this set the majority (520, or 83.1%) were screened by FISH for the presence of autonomous mini-chromosomes. Out of the 520 low copy events screened by FISH 202 (or 38.8%) transgenic events were shown to contain an autonomous mini-chromosome (column “% A+AI Events”), of which 75 (or 14.4%) contained no integrated copies in addition to the autonomous molecule (column “% A only Events”).

TABLE 24
MC constructs containing 5 gene stack tested in sugar cane callus

| | | | | % Low | | |
|---|---|---|---|---|---|---|
| CHROM# 2 | CHROM# 5 | # G418R | % NPTII | copy | % A + AI | % A Only |
| gene stack¹ | gene stack² | events³ | positive⁴ | (0.5-3.0)⁵ | Events⁶ | Events⁷ |
| 5802 | 6000 | 99 | 95% | 24% | 48% | 10% |
| 5815 | 6002 | 191 | 91% | 19% | 28% | 3% |
| 5817 | 6003 | 75 | 95% | 19% | 67% | 18% |
| 5823 | 6006 | 87 | 90% | 21% | 47% | 7% |
| 5810 | 6018 | 186 | 84% | 15% | 43% | 14% |
| 5839 | 6020 | 147 | 91% | 13% | 50% | 21% |
| 5820 | 6022 | 245 | 68% | 25% | 54% | 27% |
| 5824 | 6023 | 118 | 91% | 19% | 56% | 28% |
| 5860 | 6024 | 149 | 89% | 19% | 19% | 14% |
| 5800 | 6025 | 150 | 88% | 22% | 18% | 0% |
| 5821 | 6026 | 92 | 88% | 22% | 25% | 13% |
| 5834 | 6027 | 120 | 78% | 16% | 31% | 0% |
| 5829 | 6028 | 159 | 84% | 20% | 45% | 27% |
| 5819 | 6029 | 168 | 82% | 12% | 24% | 18% |
| 5809 | 6030 | 182 | 85% | 26% | 65% | 39% |
| 5850 | 6031 | 207 | 75% | 19% | 38% | 15% |
| 5814 | 6032 | 93 | 77% | 16% | 22% | 11% |
| 5864 | 6034 | 62 | 90% | 24% | 26% | 10% |

¹CHROM# references constructs listed in tables 5, 6, 7 and 9
²CHROM# of the corresponding 5 gene stack containing construct
³total number of G418 resistant events selected from multiple bombardments
⁴the percentage of true transformed events based on NPTII PCR amplification (true positive rate)
⁵the percentage of low copy number events out of the total number of NPTII positive events
⁶the percentage of low copy number events screened by FISH that contains an autonomous MC by itself, or in combination with an integrated copy of the MC
⁷the percentage of low copy number events screened by FISH that only contains an autonomous MC and no integrated copies.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Applicant intends that the sequence listing filed herewith forms a part of the description of the specification and is hereby in corporated by reference in its entirety.

Although the foregoing invention has been described in some detail by way of illustration and example for purposes of clarity of understanding, it will be obvious that certain changes and modifications may be practiced within the scope of the appended claims.

1. A sugar cane mini-chromosome comprising an polynucleotide comprising:
(a) a nucleotide sequence that is (i) the nucleotide sequence of SEQ ID NO:7, (ii) a nucleotide sequence that is at least 95% identical to the nucleotide sequence of SEQ ID NO:7, or (iii) a nucleotide sequence that hybridizes to the complete complement of the nucleotide sequence of SEQ ID NO:7 under stringent hybridization conditions comprising 0.02 M to 0.15 M NaCl at temperatures of 50° C. to 70° C.;
(b) a nucleotide sequence that is (i) the nucleotide sequence of SEQ ID NO:6, (ii) a nucleotide sequence that is at least 95% identical to the nucleotide sequence of SEQ ID NO:6, or (iii) a nucleotide sequence that hybridizes to the complete complement of the nucleotide sequence of SEQ ID NO:6 under stringent hybridization conditions comprising 0.02 M to 0.15 M NaCl at temperatures of 50° C. to 70° C.;
(c) a nucleotide sequence that is (i) the nucleotide sequence of SEQ ID NO:1, (ii) a nucleotide sequence that is at least 95% identical to the nucleotide sequence of SEQ ID NO:1, or (iii) a nucleotide sequence that hybridizes to the complete complement of the nucleotide sequence of SEQ ID NO:1 under stringent hybridization conditions comprising 0.02 M to 0.15 M NaCl at temperatures of 50° C. to 70° C.;
(d) a nucleotide sequence that is (i) the nucleotide sequence of SEQ ID NO:2, (ii) a nucleotide sequence that is at least 95% identical to the nucleotide sequence of SEQ ID NO:2, or (iii) a nucleotide sequence that hybridizes to the complete complement of the nucleotide sequence of SEQ ID NO:2 under stringent hybridization conditions comprising 0.02 M to 0.15 M NaCl at temperatures of 50° C. to 70° C.;
(e) a nucleotide sequence that is (i) the nucleotide sequence of SEQ ID NO:12, (ii) a nucleotide sequence that is at least 95% identical to the nucleotide sequence of SEQ ID NO:12, or (iii) a nucleotide sequence that hybridizes to the complete complement of the nucleotide sequence of SEQ ID NO:12 under stringent hybridization conditions comprising 0.02 M to 0.15 M NaCl at temperatures of 50° C. to 70° C.;
(f) a nucleotide sequence that is (i) the nucleotide sequence of SEQ ID NO:9, (ii) a nucleotide sequence that is at least 95% identical to the nucleotide sequence of SEQ ID NO:9, or (iii) a nucleotide sequence that hybridizes to the complete complement of the nucleotide sequence of SEQ ID NO:9 under stringent hybridization conditions comprising 0.02 M to 0.15 M NaCl at temperatures of 50° C. to 70° C.;
(g) a nucleotide sequence that is (i) the nucleotide sequence of SEQ ID NO:11, (ii) a nucleotide sequence that is at least 95% identical to the nucleotide sequence of SEQ ID NO:11, or (iii) a nucleotide sequence that hybridizes to the complete complement of the nucleotide sequence of SEQ ID NO:11 under stringent hybridization conditions comprising 0.02 M to 0.15 M NaCl at temperatures of 50° C. to 70° C.;
(h) a nucleotide sequence that is (i) the nucleotide sequence of SEQ ID NO:5, (ii) a nucleotide sequence that is at least 95% identical to the nucleotide sequence of SEQ ID NO:5, or (iii) a nucleotide sequence that hybridizes to the complete complement of the nucleotide sequence of SEQ ID NO:5 under stringent hybridization conditions comprising 0.02 M to 0.15 M NaCl at temperatures of 50° C. to 70° C.;
(i) a nucleotide sequence that is (i) the nucleotide sequence of SEQ ID NO:8, (ii) a nucleotide sequence that is at least 95% identical to the nucleotide sequence of SEQ ID NO:8, or (iii) a nucleotide sequence that hybridizes to the complete complement of the nucleotide sequence of SEQ ID NO:8 under stringent hybridization conditions comprising 0.02 M to 0.15 M NaCl at temperatures of 50° C. to 70° C.;
(j) a nucleotide sequence that is (i) the nucleotide sequence of SEQ ID NO:3, (ii) a nucleotide sequence that is at least 95% identical to the nucleotide sequence of SEQ ID NO:3, or (iii) a nucleotide sequence that hybridizes to the complete complement of the nucleotide sequence of SEQ ID NO:3 under stringent hybridization conditions comprising 0.02 M to 0.15 M NaCl at temperatures of 50° C. to 70° C.;
(k) a nucleotide sequence that is (i) the nucleotide sequence of SEQ ID NO:4, (ii) a nucleotide sequence that is at least 95% identical to the nucleotide sequence of SEQ ID NO:4, or (iii) a nucleotide sequence that hybridizes to the complete complement of the nucleotide sequence of SEQ ID NO:4 under stringent hybridization conditions comprising 0.02 M to 0.15 M NaCl at temperatures of 50° C. to 70° C.; or
(l) a nucleotide sequence that is (i) the nucleotide sequence of SEQ ID NO:10, (ii) a nucleotide sequence that is at least 95% identical to the nucleotide sequence of SEQ ID NO:10, or (iii) a nucleotide sequence that hybridizes to the complete complement of the nucleotide sequence of SEQ ID NO:10 under stringent hybridization conditions comprising 0.02 M to 0.15 M NaCl at temperatures of 50° C. to 70° C.

2. The sugar cane mini-chromosome of claim 1, wherein the polynucleotide comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12.

3. A sugar cane mini-chromosome comprising an array comprising at least two copies of a polynucleotide comprising polynucleotide selected from the group consisting of:
(a) a nucleotide sequence that is (i) the nucleotide sequence of SEQ ID NO:7, (ii) a nucleotide sequence that is at least 95% identical to the nucleotide sequence of SEQ ID NO:7, or (iii) a nucleotide sequence that hybridizes to the complete complement of the nucleotide sequence of SEQ ID NO:7 under stringent hybridization conditions comprising 0.02 M to 0.15 M NaCl at temperatures of 50° C. to 70° C.;
(b) a nucleotide sequence that is (i) the nucleotide sequence of SEQ ID NO:6, (ii) a nucleotide sequence that is at least 95% identical to the nucleotide sequence of SEQ ID NO:6, or (iii) a nucleotide sequence that hybridizes to the complete complement of the nucleotide sequence of SEQ ID NO:6 under stringent hybridization conditions comprising 0.02 M to 0.15 M NaCl at temperatures of 50° C. to 70° C.;
(c) a nucleotide sequence that is (i) the nucleotide sequence of SEQ ID NO:1, (ii) a nucleotide sequence that is at least 95% identical to the nucleotide sequence of SEQ ID NO:1, or (iii) a nucleotide sequence that hybridizes to the complete complement of the nucleotide sequence of SEQ ID NO:1 under stringent hybridization conditions comprising 0.02 M to 0.15 M NaCl at temperatures of 50° C. to 70° C.;
(d) a nucleotide sequence that is (i) the nucleotide sequence of SEQ ID NO:2, (ii) a nucleotide sequence that is at least 95% identical to the nucleotide sequence of SEQ ID NO:2, or (iii) a nucleotide sequence that hybridizes to the complete complement of the nucleotide sequence of SEQ ID NO:2 under stringent hybridization conditions comprising 0.02 M to 0.15 M NaCl at temperatures of 50° C. to 70° C.;
(e) a nucleotide sequence that is (i) the nucleotide sequence of SEQ ID NO:12, (ii) a nucleotide sequence that is at least 95% identical to the nucleotide sequence of SEQ ID NO:12, or (iii) a nucleotide sequence that hybridizes to the complete complement of the nucleotide sequence of SEQ ID NO:12 under stringent hybridization conditions comprising 0.02 M to 0.15 M NaCl at temperatures of 50° C. to 70° C.;
(f) a nucleotide sequence that is (i) the nucleotide sequence of SEQ ID NO:9, (ii) a nucleotide sequence that is at least 95% identical to the nucleotide sequence of SEQ ID NO:9, or (iii) a nucleotide sequence that hybridizes to the complete complement of the nucleotide sequence of SEQ ID NO:9 under stringent hybridization conditions comprising 0.02 M to 0.15 M NaCl at temperatures of 50° C. to 70° C.;
(g) a nucleotide sequence that is (i) the nucleotide sequence of SEQ ID NO:11, (ii) a nucleotide sequence that is at least 95% identical to the nucleotide sequence of SEQ ID NO:11, or (iii) a nucleotide sequence that hybridizes to the complete complement of the nucleotide sequence of SEQ ID NO:11 under stringent hybridization conditions comprising 0.02 M to 0.15 M NaCl at temperatures of 50° C. to 70° C.;
(h) a nucleotide sequence that is (i) the nucleotide sequence of SEQ ID NO:5, (ii) a nucleotide sequence that is at least 95% identical to the nucleotide sequence of SEQ ID NO:5, or (iii) a nucleotide sequence that hybridizes to the complete complement of the nucleotide sequence of SEQ ID NO:5 under stringent hybridization conditions comprising 0.02 M to 0.15 M NaCl at temperatures of 50° C. to 70° C.;
(i) a nucleotide sequence that is (i) the nucleotide sequence of SEQ ID NO:8, (ii) a nucleotide sequence that is at least 95% identical to the nucleotide sequence of SEQ ID NO:8, or (iii) a nucleotide sequence that hybridizes to the complete complement of the nucleotide sequence of SEQ ID NO:8 under stringent hybridization conditions comprising 0.02 M to 0.15 M NaCl at temperatures of 50° C. to 70° C.;
(j) a nucleotide sequence that is (i) the nucleotide sequence of SEQ ID NO:3, (ii) a nucleotide sequence that is at least 95% identical to the nucleotide sequence of SEQ ID NO:3, or (iii) a nucleotide sequence that hybridizes to the complete complement of the nucleotide sequence of SEQ ID NO:3 under stringent hybridization conditions comprising 0.02 M to 0.15 M NaCl at temperatures of 50° C. to 70° C.;
(k) a nucleotide sequence that is (i) the nucleotide sequence of SEQ ID NO:4, (ii) a nucleotide sequence that is at least 95% identical to the nucleotide sequence of SEQ ID NO:4, or (iii) a nucleotide sequence that hybridizes to the complete complement of the nucleotide sequence of SEQ ID NO:4 under stringent hybridization conditions comprising 0.02 M to 0.15 M NaCl at temperatures of 50° C. to 70° C.;
(l) a nucleotide sequence that is (i) the nucleotide sequence of SEQ ID NO:10, (ii) a nucleotide sequence that is at least 95% identical to the nucleotide sequence of SEQ ID NO:10, or (iii) a nucleotide sequence that hybridizes to the complete complement of the nucleotide sequence of SEQ ID NO:10 under stringent hybridization conditions comprising 0.02 M to 0.15 M NaCl at temperatures of 50° C. to 70° C.,
(j) a nucleotide sequence comprising SEQ ID NO: 1,
(k) a nucleotide sequence comprising SEQ ID NO:2,
(l) a nucleotide sequence comprising SEQ ID NO: 3,
(m) a nucleotide sequence comprising SEQ DI NO: 4,
(n) a nucleotide sequence comprising SEQ ID NO: 5,
(o) a nucleotide sequence comprising SEQ ID NO: 6,
(p) a nucleotide sequence comprising SEQ ID NO: 7,
(q) a nucleotide sequence comprising SEQ ID NO: 8,
(r) a nucleotide sequence comprising SEQ ID NO: 9,
(s) a nucleotide sequence comprising SEQ ID NO: 10,
(t) a nucleotide sequence comprising SEQ ID NO: 11, or
(u) a nucleotide sequence comprising SEQ ID NO: 12.

4. A sugar cane mini-chromosome of claim 3, wherein the array comprises at least ten copies of the polynucleotide.

5. A sugar cane mini-chromosome of claim 3, wherein the array comprises at least 25 copies of the polynucleotide.

6. (canceled)

7. A sugar cane mini-chromosome of claim 3, wherein the array comprises from 2 to 1000 copies of the polynucleotide.

8. (canceled)

9. The sugar cane mini-chromosome of claim 3, wherein the array is from 1 to 250 kb in length.

10-16. (canceled)

17. The sugar cane mini-chromosome of claim 1, wherein the sugar cane mini-chromosome comprises a plurality of restriction sites for insertion of an exogenous nucleic acid.

18. The sugar cane mini-chromosome of claim 1, wherein the sugar cane mini-chromosome comprises an exogenous nucleic acid.

19. The sugar cane mini-chromosome of claim 18, wherein the sugar cane mini-chromosome comprises at least three exogenous nucleic acids.

20. The sugar cane mini-chromosome of claim 19, wherein at least one exogenous nucleic acid is operably linked to a heterologous regulatory sequence functional in a sugar cane plant cell.

21. The sugar cane mini-chromosome of claim 18, wherein the exogenous nucleic acid is an herbicide resistance gene, a nitrogen fixation gene, an insect resistance gene, a disease resistance gene, a plant stress-induced gene, a nutrient utilization gene, a gene that affects plant pigmentation, a gene that encodes an antisense or ribozyme molecule, a gene encoding a secretable antigen, a toxin gene, a receptor gene, a ligand gene, a seed storage gene, a hormone gene, an enzyme gene, an antibody gene, a growth factor gene, a drought resistance gene, a heat resistance gene, a chilling resistance gene, a freezing resistance gene, an excessive moisture resistance gene, a salt stress resistance gene or a biofuel gene.

22. The sugar cane mini-chromosome of claim 1, wherein the sugar cane mini-chromosome exhibits a mitotic segregation efficiency in sugar cane plant cells of at least 90%

23. A vector comprising the polynucleotide selected from the group consisting of:
(a) a nucleotide sequence that is (i) the nucleotide sequence of SEQ ID NO:7, (ii) a nucleotide sequence that is at least 95% identical to the nucleotide sequence of SEQ ID NO:7, or (iii) a nucleotide sequence that hybridizes to the complete complement of the nucleotide sequence of SEQ ID NO:7 under stringent hybridization conditions comprising 0.02 M to 0.15 M NaCl at temperatures of 50° C. to 70° C.;
(b) a nucleotide sequence that is (i) the nucleotide sequence of SEQ ID NO:6, (ii) a nucleotide sequence that is at least 95% identical to the nucleotide sequence of SEQ ID NO:6, or (iii) a nucleotide sequence that hybridizes to the complete complement of the nucleotide sequence of SEQ ID NO:6 under stringent hybridization conditions comprising 0.02 M to 0.15 M NaCl at temperatures of 50° C. to 70° C.;
(c) a nucleotide sequence that is (i) the nucleotide sequence of SEQ ID NO:1, (ii) a nucleotide sequence that is at least 95% identical to the nucleotide sequence of SEQ ID NO:1, or (iii) a nucleotide sequence that hybridizes to the complete complement of the nucleotide sequence of SEQ ID NO:1 under stringent hybridization conditions comprising 0.02 M to 0.15 M NaCl at temperatures of 50° C. to 70° C.;
(d) a nucleotide sequence that is (i) the nucleotide sequence of SEQ ID NO:2, (ii) a nucleotide sequence that is at least 95% identical to the nucleotide sequence of SEQ ID NO:2, or (iii) a nucleotide sequence that hybridizes to the complete complement of the nucleotide sequence of SEQ ID NO:2 under stringent hybridization conditions comprising 0.02 M to 0.15 M NaCl at temperatures of 50° C. to 70° C.;
(e) a nucleotide sequence that is (i) the nucleotide sequence of SEQ ID NO:12, (ii) a nucleotide sequence that is at least 95% identical to the nucleotide sequence of SEQ ID NO:12, or (iii) a nucleotide sequence that hybridizes to the complete complement of the nucleotide sequence of SEQ ID NO:12 under stringent hybridization conditions comprising 0.02 M to 0.15 M NaCl at temperatures of 50° C. to 70° C.;
(f) a nucleotide sequence that is (i) the nucleotide sequence of SEQ ID NO:9, (ii) a nucleotide sequence that is at least 95% identical to the nucleotide sequence of SEQ ID NO:9, or (iii) a nucleotide sequence that hybridizes to the complete complement of the nucleotide sequence of SEQ ID NO:9 under stringent hybridization conditions comprising 0.02 M to 0.15 M NaCl at temperatures of 50° C. to 70° C.;
(g) a nucleotide sequence that is (i) the nucleotide sequence of SEQ ID NO:11, (ii) a nucleotide sequence that is at least 95% identical to the nucleotide sequence of SEQ ID NO:11, or (iii) a nucleotide sequence that hybridizes to the complete complement of the nucleotide sequence of SEQ ID NO:11 under stringent hybridization conditions comprising 0.02 M to 0.15 M NaCl at temperatures of 50° C. to 70° C.;
(h) a nucleotide sequence that is (i) the nucleotide sequence of SEQ ID NO:5, (ii) a nucleotide sequence that is at least 95% identical to the nucleotide sequence of SEQ ID NO:5, or (iii) a nucleotide sequence that hybridizes to the complete complement of the nucleotide sequence of SEQ ID NO:5 under stringent hybridization conditions comprising 0.02 M to 0.15 M NaCl at temperatures of 50° C. to 70° C.;
(i) a nucleotide sequence that is (i) the nucleotide sequence of SEQ ID NO:8, (ii) a nucleotide sequence that is at least 95% identical to the nucleotide sequence of SEQ ID NO:8, or (iii) a nucleotide sequence that hybridizes to the complete complement of the nucleotide sequence of SEQ ID NO:8 under stringent hybridization conditions comprising 0.02 M to 0.15 M NaCl at temperatures of 50° C. to 70° C.;
(j) a nucleotide sequence that is (i) the nucleotide sequence of SEQ ID NO:3, (ii) a nucleotide sequence that is at least 95% identical to the nucleotide sequence of SEQ ID NO:3, or (iii) a nucleotide sequence that hybridizes to the complete complement of the nucleotide sequence of SEQ ID NO:3 under stringent hybridization conditions comprising 0.02 M to 0.15 M NaCl at temperatures of 50° C. to 70° C.;
(k) a nucleotide sequence that is (i) the nucleotide sequence of SEQ ID NO:4, (ii) a nucleotide sequence that is at least 95% identical to the nucleotide sequence of SEQ ID NO:4, or (iii) a nucleotide sequence that hybridizes to the complete complement of the nucleotide sequence of SEQ ID NO:4 under stringent hybridization conditions comprising 0.02 M to 0.15 M NaCl at temperatures of 50° C. to 70° C.;
(l) a nucleotide sequence that is (i) the nucleotide sequence of SEQ ID NO:10, (ii) a nucleotide sequence that is at least 95% identical to the nucleotide sequence of SEQ ID NO:10, or (iii) a nucleotide sequence that hybridizes to the complete complement of the nucleotide sequence of SEQ ID NO:10 under stringent hybridization conditions comprising 0.02 M to 0.15 M NaCl at temperatures of 50° C. to 70° C.,
(j) a nucleotide sequence comprising SEQ ID NO: 1,
(k) a nucleotide sequence comprising SEQ ID NO:2,
(l) a nucleotide sequence comprising SEQ ID NO: 3,
(m) a nucleotide sequence comprising SEQ DI NO: 4,
(n) a nucleotide sequence comprising SEQ ID NO: 5,
(O) a nucleotide sequence comprising SEQ ID NO: 6,
(p) a nucleotide sequence comprising SEQ ID NO: 7,
(q) a nucleotide sequence comprising SEQ ID NO: 8,
(r) a nucleotide sequence comprising SEQ ID NO: 9,
(s) a nucleotide sequence comprising SEQ ID NO: 10,
(t) a nucleotide sequence comprising SEQ ID NO: 11, or
(u) a nucleotide sequence comprising SEQ ID NO: 12.

24-25. (canceled)

26. A vector comprising the sugar cane mini-chromosome of claim 1

27-29. (canceled)

30. A cell comprising the sugar cane mini-chromosome of claim 1.

31. A cell comprising the vector of claim 26.

32. The cell of claim 30, wherein the cell is a sugar cane plant cell.

33. The sugar cane plant cell of claim 32 comprising a sugar cane mini-chromosome, wherein the sugar cane mini-chromosome is not integrated into the genome of the sugar cane plant cell.

34. The sugar cane plant cell of claim 32 comprising a sugar cane mini-chromosome that comprises an exogenous nucleic acid, wherein the sugar cane plant cell exhibits an altered phenotype associated with the expression of the exogenous nucleic acid.

35. The sugar cane plant cell of claim 34, wherein the altered phenotype comprises altered expression of a native gene.

36. The sugar cane plant cell of claim 34, wherein the altered phenotype comprises altered expression of an exogenous gene.

37. A sugar cane plant tissue comprising the sugar cane plant cell of claim 32.

38. A sugar cane plant comprising the sugar cane plant cell of claim 32.

39. A sugar cane plant part comprising the sugar cane plant cell of claim 32.

40. A sugar cane seed obtained from the sugar cane plant of claim 38.

41. A sugar cane plant progeny comprising a sugar cane mini-chromosome, wherein the plant progeny is the result of breeding a plant of claim 38 comprising the sugar cane mini-chromosome.

42. A method of using a sugar cane plant of claim 38, wherein the sugar cane plant comprises a sugar cane mini-chromosome comprising an exogenous nucleic acid encoding a recombinant protein, the method comprising growing the plant to produce the recombinant protein.

43. The method of claim 42, further comprising the step of harvesting or processing the sugar cane plant.
